(19) United States
(12) Patent Application Publication
(10) Pub. No.: US 2022/0259551 A1
(43) Pub. Date: Aug. 18, 2022

(54) METHODS OF OPTIMIZED EUGLENA FERMENTATION USING ENGINEERED TANK DESIGN

(71) Applicant: NOBLEGEN INC., Peterborough (CA)

(72) Inventors: Adam J. NOBLE, Peterborough, ON (CA); Mostafa Zahid SHARIF, Peterborough, ON (CA); Bijaya UPRETY, Peterborough, ON (CA); Lee Anthony CASTIGLIONE, Peterborough, ON (CA); Paul-Philippe CHAMPAGNE, Peterborough, ON (CA); Ryan Richard CULLEN, Peterborough, ON (CA); Alexander John BAYRAK, Peterborough, ON (CA); Scott Cameron FARROW, Peterborough, ON (CA)

(21) Appl. No.: 17/618,936

(22) PCT Filed: Jun. 29, 2020
(86) PCT No.: PCT/IB2020/056135

§ 371 Date: Dec. 14, 2021

Related U.S. Application Data
(60) Provisional application No. 62/868,343, filed on Jun. 28, 2019.
(60) Provisional application No. 62/868,589, filed on Jun. 28, 2019.
(60) Provisional application No. 62/954,837, filed on Dec. 30, 2019.

(51) Int. Cl. C12N 1/12; C12M 1/00; C12M 1/34

(52) U.S. Cl. CPC C12N 1/12; C12M 29/06; C12N 2500/02; C12M 41/26; C12M 41/34; C12M 41/12

(57) ABSTRACT

Embodiments herein are directed to methods of heterotrophically culturing a. Embodiments herein are directed to methods, systems, and bioreactors for heterotrophically culturing Euglena sp. microorganism, a Schizochytrium sp. microorganism, or a Chlorella sp. microorganism comprising: culturing the microorganism in a culture media containing one or more carbon source, one or more nitrogen source, and one or more salt; maintaining a pH of between about 2.0 to about 4.0; maintaining a temperature of about 20° C. to about 30° C.; and maintaining an environment with substantially no light; wherein the culturing occurs in three cultivation stages.

### CROSS-REFERENCE TO RELATED APPLICATIONS

This application claims the benefit of U.S. Provisional Application No. 62/868,343 filed Jun. 28, 2019, U.S. Provisional Application No. 62/868,589 filed Jun. 28, 2019, and U.S. Provisional Application No. 62/954,837 filed Dec. 30, 2019, each of which are hereby incorporated by reference in their entirety.

### SUMMARY

Embodiments described herein relate to processes for culturing a Euglena sp. microorganism, a Schizochytrium sp. microorganism, or a Chlorella sp. microorganism.

Embodiments described herein are directed to bioreactor for heterotrophically growing microorganisms, comprising a tank configured to receive culture media and ingredients for growing the heterotrophic microorganisms; an air supply system configured to introduce a gas into the tank, mixing the culture media and microorganisms within the tank, wherein the air supply system includes a lower pressure supply device and a higher pressure supply device

Embodiments described herein are directed to a method of heterotrophically culturing a Euglena gracilis comprising: culturing the Euglena gracilis in a culture media containing one or more carbon source, one or more nitrogen source, and one or more salt; maintaining a pH of between about 2.0 to about 4.0; maintaining a temperature of about 20° C. to about 30° C.; and maintaining an environment with substantially no light; wherein the culturing occurs in three cultivation stages

Euglena gracilis, a unicellular phyloflagellate protist, can easily metabolize carbon (e.g., glucose and fructose) and nitrogen (e.g., corn steep liquor, yeast extract and inorganic nitrogen sources) for cell growth, and produce various metabolites (e.g., protein, paramylon/β-1, 3 glucan, and lipid). Due to its unique potential in biotech and food industries, research has been conducted to cultivate this microorganism at large scale for the production of polyunsaturated fatty acids, protein, and paramylon used in food, beverage, nutraceutical, and biofuel production.

It has been demonstrated that medium optimization is very critical for developing a bioprocess, as it influences the yield of targeted products and their cost of production. Hence, there is a great need for optimization of medium components to support the growth of the microorganism while producing products of interest.

E. gracilis has been grown photoautotrophically (i.e., synthesis of sugar and other organic molecules in presence of light and CO₂) in flask, photobioreactor, and raceway pond systems. However, the open pond system is not suitable for cultivation of Euglena due to limitations in controlling contaminations and cultivation parameters. Likewise, although the concentration of growth nutrients and cultivation parameters can be maintained precisely in photobioreactors, the use of photoautotrophic approach for growing this microalgae in large scale has been limited by the technical challenges for scale up and the high cost for running large scale photobioreactors sterilely. Because the yield of biomass through phototrophic cultivation of Euglena is very low due to light limitation, heterotrophic cultivation has been considered as a method of choice in industry. However, there remains a great need for more robust fermentation processes that could be scalable to a manufacturing scale while maintaining high yield and productivity obtained at the bench scale.

The present invention is directed to overcoming these and other deficiencies in the art.

Accordingly, the present application includes a method of heterotrophically culturing a Euglena sp. microorganism, a Schizochytrium sp. microorganism, or a Chlorella sp. microorganism comprising:
a first step of batch culturing the Euglena sp. microorganism, Schizochytrium sp. microorganism, or Chlorella sp. microorganism in a first culture medium containing one or more carbon source, one or more nitrogen source, and one or more salt; and
a second step of fed-batch culturing the Euglena sp. microorganism, Schizochytrium sp. microorganism, or Chlorella sp. microorganism with a second culture medium containing one or more carbon source, one or more nitrogen source, and one or more salt.

In another embodiment, the method further comprises a third step of continuously culturing the microorganism with a third culture medium containing one or more carbon source, one or more nitrogen source, and one or more salt.

Another aspect of embodiments described herein is culture media as described herein.

Yet another aspect of the present application is a bioreactor for heterotrophically growing microorganisms. The bioreactor includes a tank configured to receive culture media and ingredients for growing the heterotrophic microorganisms; an air supply system configured to introduce a gas into the tank, mixing the culture media and microorganisms within the tank, wherein the air supply system includes a lower pressure supply device and a higher pressure supply device.

Yet a further aspect of the present application is a system for producing a biomass. The system includes a plurality of bioreactors connected in parallel, each bioreactor including an individual tank; a plurality of input systems configured to provide culture media, microorganisms, and ingredients individually to each of the bioreactor tanks; an air supply system configured to introduce a gas into each of the bioreactor tanks, where the air supply system includes a lower pressure supply device and a higher pressure supply device.

Yet a further aspect of the present application is a method of heterotrophically culturing a microorganism. This method involves culturing the microorganism in a culture media containing one or more carbon source, one or more nitrogen source, one or more sugar, one or more alcohol, one or more oil, and one or more salt; maintaining a pH of between about 2.0 to about 4.0; maintaining a temperature of about 20° C. to about 30° C.; and maintaining an environment with substantially no light; where the culturing occurs within a tank configured to receive the culture media, an air supply system configured to introduce a gas into the tank, an ability to mix the culture media and microorganisms within the tank, wherein the air supply system includes a lower pressure supply device and a higher pressure supply device.

### DESCRIPTION OF THE DRAWINGS

FIG. 1 depicts E. gracilis growth characteristics of the fermentation of Example 3.

FIG. 2 depicts E. gracilis growth characteristics of the fermentation of Example 4.

FIG. 3 represents the 100% fresh media control in Example 6. Time (h) of each cycle is on the x-axis while the y-axis represents the DCW (g/L), OD (600 nm), pH, and cell count (cells/mL).

FIG. 4 represents the glucose supplemented 50% recycled hybrid media in Example 6. Time (h) of each cycle is on the x-axis while the y-axis represents the DCW (g/L), OD (600 nm), pH, and cell count (cells/mL).

FIGS. 5A and 5B are graphs representing nutrient profiles of media. FIG. 5A represents 100% fresh growth media whereas FIG. 5B represents the glucose supplemented 50% recycled hybrid media nutrient levels over time. x-axis represents time in hours, with cycle 1, 2 and 3 indicated. y-axis represents the concentration in g/L of glucose, ammonium, ammonium sulfate, and potassium in the supernatant.

FIG. 6 represents the 100% fresh media control bioreactor in Example 7. Incubation time (h) of each phase is on the x-axis while the y-axis represents the DCW (g/L), OD (600 nm), pH, glucose (g/L) and cell count (cells/mL). Culturing phase (batch, fed-batch or continuous) is labelled below the figure.

FIG. 7 represents the recycled hybrid media bioreactor in Example 7. Incubation time (h) of each phase is on the x-axis while the y-axis represents the DCW (g/L), OD (600 nm), pH, glucose (g/L) and cell count (cells/mL). Culturing phase (batch, fed-batch or continuous) is labelled below the figure.

FIG. 8 depicts growth data during continuous fermentation of E. gracilis using hybrid medium.

FIG. 9 depicts major cultivation parameters for continuous fermentation of E. gracilis using hybrid medium.

FIG. 10 depicts feeding, harvesting, and productivity trend during continuous fermentation of E. gracilis using hybrid medium

FIG. 11 depicts off-gas data trend during continuous fermentation of E. gracilis using hybrid medium.

FIG. 12 depicts metabolites profiling by CEDEX bioanalyzer in samples collected during continuous fermentation of E. gracilis using hybrid medium

FIG. 13 depicts growth data of the control during continuous fermentation of E. gracilis using fresh medium.

FIG. 14 depicts major cultivation parameters for the control continuous fermentation of E. gracilis using fresh medium.

FIG. 15 depicts feeding, harvesting, and productivity trend of the control during continuous fermentation of E. gracilis using fresh medium.

FIG. 16 depicts off-gas data trend of the control during continuous fermentation of E. gracilis using fresh medium.

FIG. 17 depicts metabolites profiling by CEDEX bioanalyzer in samples collected in the control during continuous fermentation of E. gracilis using fresh medium

FIG. 18 is a bar graph showing the conversion efficiency (% wt) and biomass yield/gm of carbon at the end of 48 h with lower concentration of acids (0.0005-0.05 g/L).

FIG. 19 is a bar graph showing net consumption of acid during fermentation over a 48 h time period with the use of low acids concentration (0.0005-0.05 g/L).

FIG. 20 is a graph depicting change in glucose concentration over time with low level of acids (0.0005-0.05 g/L).

FIG. 21, graphs A-E show the change in acid concentrations over time (higher acid concentrations, 2-5 g/L) (22A, Pyruvate; 22B, Malate; 22C, Lactate; 22D, Succinate; 22E, Fumarate).

FIG. 22 is a bar graph showing a comparison of net glucose consumption at the end of 48 h in presence of low and higher concentrations of acids in the glucose (15 g/L) containing media.

FIG. 23 is a graph showing change in glucose concentration over time with high levels of acids (2-5 g/L).

FIG. 24 is a graph showing net biomass change (g/L) during fermentation when higher concentrations of acids are used solely or in combination with glucose.

FIG. 25 is a bar graph showing a comparison of biomass contributions from acid portions between sole acid as a carbon source or along with glucose during the fermentation when higher acid concentrations were used.

FIG. 26 is a schematic representation of the metabolic pathways utilized by the different inputs consumed by E. gracilis during fermentation and the potential outputs.

FIG. 27 is a schematic view of a bioreactor system, including a plurality of bioreactor tanks, consistent with disclosed embodiments;

FIG. 28 is a schematic cross-sectional view of an exemplary bioreactor tanks, consistent with disclosed embodiments;

FIG. 29 is a top-view of sparger grid that may be used in combination with the bioreactor tank of FIG. 28, consistent with disclosed embodiments; and

FIG. 30 is a table showing the results of production tests using fine and coarse spargers in large production bioreactor tanks.

FIG. 31 represents the 300 L bioreactor tank in Example 14. Time (h) of the run is on the x-axis while the y-axis represents the DCW (g/L), specific consumption rates (mg/g, DCW/h). Productivity (g/L/h) and specific growth rate (μ, 1/h).

FIG. 32 represents the 300 L bioreactor tank in Example 14. Time (h) of the run is on the x-axis while the y-axis represents the DCW (g/L), Glucose concentration (g/L), feed rate (L/h), and volume (L).

FIG. 33 represents the 300 L bioreactor tank in Example 14. Time (h) of run is on the x-axis while the y-axis represents the agitation (RPM), pH, DO (%) and airflow (slpm).

FIG. 34 represents the 7000 L bioreactor tank in Example 14. Time (h) of the batch is on the x-axis while the y-axis represents the DCW (g/L), Glucose concentration (g/L) and the total DCW (kg).

FIG. 35 represents the 7000 L bioreactor tank in Example 14. Time (h) of the batch is on the x-axis while the y-axis represents the DCW (g/L), specific consumption rates (mg/g, DCW/h). Productivity (g/L/h) and specific growth rate (μ, 1/h).

FIG. 36 represents the 7000 L bioreactor tank in Example 14. Time (h) of run is on the x-axis while the y-axis represents the agitation (RPM), pH, DO (%) and airflow (m³/min).

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

As used in this application, the singular forms “a”, “an” and “the” include plural references unless the content clearly dictates otherwise. Thus, for example, reference to a “cell” includes a single cell as well as two or more of the same or different cells.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein. Terms of degree such as “substantially”, “about” and “approximately” as used herein mean a reasonable amount of deviation of the modified term such that the end result is not significantly changed.

The term “and/or” as used herein means that the listed items are present, or used, individually or in combination. In effect, this term means that “at least one of” or “one or more” of the listed items is used or present.

The term “batch” culturing refers to culturing wherein cells are allowed to consume all of the media until growth stops, typically about 2 days.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “continuous” culturing refers to the method of culturing wherein a volume of cells and media are removed from the culture, cells are harvested, and new media replaces what was removed. Continuous culturing allows for an optimized production of Euglena as well as reducing waste. Feeding is based on the consumption rate and harvest at the same rate as the growth, allowing the exponential growth phase to be extended, i.e. the amount of media put into the system matches the amount harvested or removed from the system. The advantage of using a continuous system is that is able to be automated even at a large scale of production and limits human error.

The term “centrate” or the phrase “spent growth media” refers to the media that has been used for cell culture, i.e. culture media that has a lower level of growth components in it then at the start of culturing. Spent growth media is also determined by the content of carbohydrate in the media after being used for culturing cells.

The terms “feed” and “feeding,” as used herein in relationship to Euglena culturing refer to the addition of nutrient containing medium to the culture.

The term “batch fermentation,” as used herein, refers to a process of cultivating microorganisms in a vessel filled with carbon and energy sources without addition to, or removal of, a major substrate or product stream until the process is complete. The term “batch cultivating,” as used herein, refers to cultivating by batch fermentation.

The term “fed-batch fermentation,” as used herein, refers to a process of cultivating microorganisms in a vessel which is frequently or continuously fed with a feed solution containing growth limiting nutrients, without the removal of culture fluid. Therefore, the volume of culture increases over time. The term “fed-batch cultivating,” as used herein, refers to cultivating by fed-batch fermentation.

The term “harvested culture” refers to the concentrated cells separated from some or all of the culture media. The harvested culture can be used to inoculate another bioreactor or used in downstream processing to produce isolated biomass or purified oil, protein, beta-glucan, or other component.

The term “harvesting,” as used herein, with respect to, e.g., Euglena cultures refers to separating Euglena cells from some or all of the culture media. The term “harvested culture” refers to the separated, e.g., Euglena cells.

The term “suitable” as used herein means that the selection of the particular compound or conditions would depend on the specific synthetic manipulation to be performed, and the identity of the molecule(s) to be transformed, but the selection would be well within the skill of a person trained in the art.

Where a range of values is provided, it is intended that each intervening value between the upper and lower limit of that range and any other stated or intervening value in that stated range is encompassed within the disclosure. For example, if a range of 1 mL to 8 mL is stated, it is intended that 2 mL, 3 mL, 4 mL, 5 mL, 6 mL, and 7 mL are also explicitly disclosed, as well as the range of values greater than or equal to 1 mL and the range of values less than or equal to 8 mL.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The terms “heterotrophic,” “heterotrophic environment,” or derivatives, as used herein, refers to an organism, such as an microorganism including Euglena, which is under conditions such that it obtains nutrients substantially entirely from exogenous sources of organic carbon, such as carbohydrates, lipids, alcohols, carboxylic acids, sugar alcohols, proteins, or combinations thereof. For example, Euglena is a heterotroph where it is in an environment where there is substantially no light.

The term “phototrophic” or derivatives, as used herein, refers to an organism, such as a microorganism including Euglena, when it is under a condition that it can carry out photon capture to acquire energy. For example, when an organism is phototrophic, it carries out photosynthesis to produce energy.

The term “mother culture” as used herein refers to a culture of cells that is continuously grown over time with media and cells removed or replenished on a schedule independent of the experimental conditions described herein.

“Cultivate,” “culture,” and “ferment,” and variants thereof, mean the intentional fostering of growth and/or propagation of one or more cells, such as Euglena gracilis, by use of culture conditions. Intended conditions exclude the growth and/or propagation of microorganisms in nature (without direct human intervention). The term “cultivated”, and variants thereof, refer to the intentional fostering of growth (increases in cell size, cellular contents, and/or cellular activity) and/or propagation (increases in cell numbers via mitosis) of one or more cells by use of intended culture conditions. The combination of both growth and propagation may be termed proliferation. The one or more cells may be those of a microorganism, such as Euglena gracilis. Examples of intended conditions include the use of a defined medium (with known characteristics such as pH, ionic strength, and carbon source), specified temperature, oxygen tension, carbon dioxide levels, and growth in a bioreactor.

“Dry weight” and “dry cell weight” mean weight determined in the relative absence of water. For example, reference to microalgal biomass as comprising a specified percentage of a particular component by dry weight means that the percentage is calculated based on the weight of the biomass after substantially all water has been removed. One measure of dry weight is gram dry biomass produced per liter (gDCW/L).

“Growth” means an increase in cell size, total cellular contents, and/or cell mass or weight of an individual cell, including increases in cell weight due to conversion of a fixed carbon source into intracellular oil.

“Increased lipid yield” means an increase in the lipid/oil productivity of a microalgal culture that can achieved by, for example, increasing the dry weight of cells per liter of culture, increasing the percentage of cells that contain lipid, and/or increasing the overall amount of lipid per liter of culture volume per unit time.

“Microalgal biomass,” “algal biomass,” and “biomass” mean a material produced by growth and/or propagation of microalgal cells. Biomass may contain cells and/or intracellular contents as well as extracellular material. Extracellular material includes, but is not limited to, compounds secreted by a cell.

“Microalgal flour” is a dry, particulate composition, fit for human consumption, comprising cells of microalgae, e.g., Euglena.

“Microalgal oil” and “algal oil” mean any of the lipid components produced by microalgal cells, including triacylglycerols (“TAG”).

“Oil” means any triacylglycerol (or triglyceride oil), produced by organisms, including microalgae, other plants, and/or animals. “Oil,” as distinguished from “fat,” refers, unless otherwise indicated, to lipids that are generally liquid at ordinary room temperatures and pressures. For example, “oil” includes vegetable or seed oils derived from plants, including without limitation, an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, opium poppy, castor beans, pecan, jojoba, jatropha, macadamia, Brazil nuts, and avocado, as well as combinations thereof.

“Proliferation” means a combination of both growth and propagation.

“Propagation” means an increase in cell number via mitosis or other cell division.

The term “substantially free” as used herein refers to the complete or near complete lack of light or a component. For example, a composition that is “substantially free” of water would either completely lack water, or so nearly completely lack water that the effect would be the same as if it completely lacked water.

“V/V” or “v/v,” in reference to proportions by volume, means the ratio of the volume of one substance in a composition to the volume of the composition. For example, reference to a composition that comprises 5% v/v microalgal oil means that 5% of the composition's volume is composed of microalgal oil (e.g., such a composition having a volume of 100 mm³ would contain 5 mm³ of microalgal oil), and the remainder of the volume of the composition (e.g., 95 mm³ in the example) is composed of other ingredients.

“W/W” or “w/w,” in reference to proportions by weight, means the ratio of the weight of one substance in a composition to the weight of the composition. For example, reference to a composition that comprises 5% w/w microalgal biomass means that 5% of the composition's weight is composed of microalgal biomass (e.g., such a composition having a weight of 100 mg would contain 5 mg of microalgal biomass) and the remainder of the weight of the composition (e.g., 95 mg in the example) is composed of other ingredients.

The term “biomass productivity,” as used herein and measured as gDCW/L/hr, is gram dry biomass produced per liter of culture per hour and is also called volumetric productivity.

The terms “chemostatic fermentation,” “chemostat fermentation,” or “continuous fermentation,” as used herein, refers to a process of cultivating microorganisms in a vessel in which the culture is continuously or semi-continuously fed with a feed solution containing growth limiting nutrients, and from which is simultaneously or immediately or soon thereafter harvested an effluent solution that contains cells, metabolites, waste products, and any unused nutrients. The vessel used as a growth container in this type of continuous culture is called a chemostat. In chemostat fermentation, the feed flow rate, substrate concentration, pH, temperature, and oxygen levels are continuously controlled. The terms “chemostatically cultivating,” “chemostat cultivating,” or “continuously cultivating,” as used herein, refer to cultivating by chemostatic fermentation, chemostat fermentation, or continuous fermentation.

The term “glucose limited cultivation,” as used herein, refers to a condition in which cell growth is limited by glucose concentration in the medium.

The term “residence time,” as used herein, is the time/duration when one bioreactor volume of feed medium is supplied into the bioreactor.

The term “specific glucose uptake rate,” as used herein, and measured by determining how much of a gram of glucose is consumed in one hour to produce 1 gram of dried biomass. The equation for determining specific glucose uptake rate is qs

$$( \frac{\frac{ggly}{gDCW}}{hr} ) = \frac{\frac{{glu}_{1} - {glu}_{0}}{{DCW}_{1}}}{t 1 - t 0} .$$

The term “specific growth rate,” as used herein, is the rate at which cell number increases in a population. The equation for determining specific growth rate is

$$μ = LN \frac{\frac{{X}_{1}}{{X}_{0}}}{t 1 - t 0} .$$

The highest rate is called μₘₐₓ and its unit is h⁻¹.

The term “washout,” as used herein, refers to when cells are replicating at a lower rate than cells are being removed during chemostat fermentation.

The abbreviation “DW” refers to distilled water.

The abbreviation “PW” refers to purified water.

The abbreviation “RPM” refers to rotations per minute.

The abbreviation “VVM” refers to the volume of air supply per volume of culture per minute.

The abbreviation “OUR” refers to oxygen uptake/utilization rate which is how many moles of O2 consumed per litre of culture per hour.

The abbreviation “CER” refers to carbon dioxide evolution rate which is how many moles of CO2 produced per litre of culture per hour.

The abbreviation “RQ” refers to respiratory quotient/coefficient where it is the ratio of the volume of carbon dioxide produced (e.g., by Euglena) to the volume of oxygen consumed by (e.g., by Euglena) during respiration.

The abbreviation “pO2” or “pO₂” refers to partial pressure of oxygen and is the concentration of oxygen in the gas phase in the head space above the liquid medium.

The abbreviation “DO” refers to dissolved oxygen and is the oxygen gas dissolved in the liquid medium.

Certain terms employed in the specification, examples and claims are collected herein. Unless defined otherwise, all technical and scientific terms used in this disclosure have the same meanings as commonly understood by one of ordinary skill in the art to which this disclosure belongs.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art. Preferences and options for a given aspect, feature, embodiment, or parameter of the invention should, unless the context indicates otherwise, be regarded as having been disclosed in combination with any and all preferences and options for all other aspects, features, embodiments, and parameters of the invention.

A specific species of algae named Euglena gracilis (hereinafter Euglena) belongs to a group of single-celled microscopic algae, that is often used as a candidate species for laboratory studies and technological applications. Euglena possess the representative features typical of eukaryotic cells such as a mitochondria, nucleus, and lysosome. Euglena can further be characterized for its long flagellum and large red eyespot. They are distinctive as they can produce their own nourishment (autotrophic) similar to plants, as well as eat and digest external food sources (heterotrophic) like animals. Euglena is a demonstrated, multifaceted model organism for study. Through optimizing the natural ability to employ singly or both modes of nourishment, Euglena can be directed to produce target compounds by adjusting key parameters in the production process. These critical adjustments can be used to enhance the natural mechanisms of the microorganism, to encourage rapid growth and the efficient conversion of valuable products with little waste production.

Euglena gracilis possesses the potential for mass cultivation by making use of its recycled materials via efficient conversion of input components to generate target output products that maximize yield, key for reducing cost to industry. It is possible to manipulate these factors pertaining to essential growth parameters like carbon and nitrogen sources as well as, light and temperature, to build a suit of conditions specific for product development of essential dietary supplements like oils and proteins. Growth optimization of Euglena gracilis for large scale production of these essential nutrients, framed in an environmental context, will help to limit waste and maximize efficiency through algal medium recycling. Albeit not simple, the need for alternative, environmentally-friendly solutions for industrial scale nutrient production is needed. Algae and its commercialized waste is well positioned to resolve this crisis—to reduce the industrial waste footprint while serving as a promising nutritious source of dietary supplements.

Euglena gracilis is grown heterotrophically using a growth medium in a bubble column bioreactor. A bubble column bioreactor is a tall cylindrical bioreactor used for the growth of suspended living cells in liquid phase using the sparging of air at the bottom to form bubbles within the liquid. The bubble generation creates the necessary liquid turbulence for the mixing. The aspect ratio of a bubble column bioreactor, the ratio of the height of the vessel to the diameter of the vessel, is typically between 4 and 6. In some embodiments, the production of Euglena gracilis cell cultures or cell expansion from the seed culture to the commercial production scale is performed in multiple growth cycle stages. This consists of growing Euglena gracilis cell cultures to a required cell density and volume in multiple stages by using a fermenter train. The starting growth media used to grow Euglena gracilis cells is formulated to optimize the growth and the target cell composition. A concentrated feed media which can be a unique combination of concentrated media ingredients, or groups of combined concentrated media ingredients of the same type, and/or individual concentrated media ingredients is fed to the culture of Euglena gracilis to increase the cell concentration in the starting growth media. The growth media that is used to grow Euglena gracilis includes one or more fermentable carbon sources, one or more non-fermentable carbon sources, one or more nitrogen sources, a combination of salts and minerals, and a combination of vitamins. The fermenter train comprises 12 bubble column bioreactors (2×250 L, 2×500 L, 8×20,000 L) in total and are connected in series from the seed fermenter to the large commercial fermenters in order of increasing capacity. The smaller 250 L and 500 L bubble column bioreactors are located in plant area and are used to bring the lab scale Euglena gracilis cultures from the lab scale, to and intermediate scale, the latter serving as an inoculum or seed culture for the commercial final stage scale in a 20,000 L bubble column bioreactor located in a plant area. Once the growth cycle is complete in the 20,000 L bubble column bioreactors, the culture is transferred first to a surge tank, and then to a large disk stack centrifuge for cell separation. The recovered cells are either incubated in a secondary aerobic or anaerobic fermentation stage or disrupted for protein or beta glucan recovery. The primary function of the primary fermentation process is the generation of the bulk ingredients which are 1,3-beta glucan, proteins, and lipids.

First Stage Cultivation of Euglena gracilis

First stage cultivation stage begins with the inoculation of 100 L to 125 L of fresh growth medium in the 250 L bubble column bioreactor. The inoculum or starting culture volume ranges between 15 L and 25 L and can be derived from the laboratory or a culture growing in a 500 L bubble column bioreactor.

The culture is grown in batch mode, that is the culture cells are consuming the nutrient and in particular the main carbon source without any external interaction with the culture. Once the lower threshold of the carbon source is reached, concentrated growth medium ingredients are fed to the culture to continue the growth or cell proliferation. The concentrated growth medium is fed to the culture at a rate that matches the specific carbon source consumption rate of the Euglena gracilis cell during exponential growth phase based on wet cell weight concentration of the culture. In certain embodiments, the lower threshold of the carbon source is from about 2 g/L to about 10 g/L, about 3 g/L to about 9 g/L, about 4 g/L to about 8 g/L, or about 5 g/L to about 7 g/L. In certain embodiments, the lower threshold of the carbon source is from about 6 g/L to about 14 g/L, about 7 g/L to about 13 g/L, about 8 g/L to about 12 g/L, or about 9 g/L to about 11 g/L.

The concentrated carbon source is fed through a dedicated concentrated carbon source feed line, the concentrated nitrogen source is fed through a dedicated concentrated nitrogen source feed line, and the concentrated salts source is fed through a dedicated concentrated salts source feed line. These dedicated concentrated ingredient feed lines are connected to the main feed line of the bubble column bioreactor through pneumatically actuated double-seat valves. The double seat valves enable the simultaneous flow of two media ingredients feed stream through the same valve without risk of cross mixing. The sterile/process water also has its own dedicated feed line to area and is connected to the bioreactor main feed line through an actuated double seat valve.

The feeding rate of the concentrated media ingredients is modulated by an actuated valve installed on the main feed line connected to the bubble column bioreactor. This valve is connected to and actuated by the local Programmable Logic Controller (PLC) with a timer that control the pulsing frequency of the valve and consequently the feeding rate of concentrated media ingredients to the bubble column bioreactor. It is the frequency of the valve opening that modulates the feeding rate of the concentrated growth media ingredients to the culture. The sequence in which the concentrated media ingredients is controlled by the distributed control system (DCS) through the actuation of the double-seat valves connecting the concentrated growth media ingredients feed lines to the bubble column bioreactor main feed line. Automatic feed of the cultures in area by feed schedule can be implemented.

The concentrated growth medium is fed to the culture to match the specific carbon source consumption of Euglena gracilis during exponential growth phase based on wet cell weight concentration of the culture. The transfer and the distribution of the concentrated media ingredients between bioreactors is performed through a double-seat valve bank.

Once the volume of the culture reaches 80 to 90% of the maximum working volume of the bioreactor, part of or the entire content of the bioreactor is aseptically transferred to the next stage bioreactor, the 500 L bubble column bioreactor via a pre-steam sterilized stainless steel braided hose transfer line (⅜″) connecting both vessels. In some embodiments, the final wet cell weight ranges between 5 to 250 g/L (1.6 to 80 g/L dry cell weight), between 5 to 80 g/L (1.6 to 25.6 g/L), or between 30 to 60 g/L wet cell weight (6.4 to 19.2 g/L dry cell weight). The 250 L bubble column bioreactor is pressurized to about 10 psi to about 15 psi and the valve to the sterile transfer hose line is open so that the culture flows from the 250 L to the 500 L bubble column bioreactor.

Second Stage Cultivation of Euglena gracilis

Second stage cultivation stage begins with the inoculation of 100 L to 200 L of fresh growth medium in the 500 L bubble column bioreactor. The inoculum culture ranges between 15 L and 50 L and originates from the laboratory or from a 250 L bioreactor. The volume of the starting culture is typically between 110 L to 125 L.

The culture is grown in batch mode, that is the culture cells are consuming the nutrients and the main carbon source without external interaction with the culture. Once the lower threshold of the carbon source is reached, concentrated growth medium ingredients are fed through dedicated feed lines to continue the growth or cell proliferation. The concentrated growth medium is fed to the culture at a rate that matches the specific carbon source consumption rate of Euglena gracilis during exponential growth phase based on wet cell weight concentration of the culture. In certain embodiments, the lower threshold of the carbon source is from about 2 g/L to about 10 g/L, about 3 g/L to about 9 g/L, about 4 g/L to about 8 g/L, or about 5 g/L to about 7 g/L. In certain embodiments, the lower threshold of the carbon source is from about 6 g/L to about 14 g/L, about 7 g/L to about 13 g/L, about 8 g/L to about 12 g/L, or about 9 g/L to about 11 g/L.

The concentrated carbon source is fed through a dedicated concentrated carbon source feed line, the concentrated nitrogen source is fed through a dedicated concentrated nitrogen source feed line, and the concentrated salts source is fed through a dedicated concentrated salts source feed line. These dedicated concentrated ingredient feed lines are connected to the main feed line connected to the bubble column bioreactor through pneumatically actuated double-seat valves. The sterile/process water also has its own dedicated feed line.

The feeding rate of the concentrated media ingredients is modulated by an actuated valve installed on the main feed line connected to the bubble column bioreactor. This valve is connected to and actuated by the local Programmable Logic Controller (PLC) with a timer that controls the pulsing frequency of the concentrated media ingredients to the bubble column bioreactor. It is the frequency of the valve opening that modulates the feeding rate of the concentrated growth media ingredients to the culture. The sequence in which the concentrated media ingredients is controlled by the distributed control system (DCS) through the actuation of the double-seat valves connecting the concentrated growth media ingredients feed lines to the bubble column bioreactor main feed line. Automatic feed of the cultures can be controlled by a feed schedule.

The concentrated growth medium is fed to the culture to match the specific carbon source consumption of Euglena gracilis during exponential growth phase based on wet cell weight concentration of the culture. The transfer and the distribution of the concentrated media ingredients from one area to the bubble column bioreactors is performed through a double-seat valve bank. The double seat valves enable the simultaneous flow of two media ingredients feed stream through the same valve without risk of cross mixing.

Once the volume of the culture reaches 80 to 90% of the maximum working volume of the bioreactor, part of or the entire content of the bioreactor is aseptically transferred to the 20,000 L bubble column bioreactors through a transfer line (2″ stainless steel pipe) equipped with a centrifugal pump. In some instances, part of or the entire content of the bioreactor is aseptically transferred to a pilot size centrifuge of the processing of small development batches. In some embodiments, the final wet cell weight ranges between 5 to 250 g/L (1.6 to 80 g/L dry cell weight), between 5 to 80 g/L (1.6 to 25.6 g/L), or between 30 to 60 g/L wet cell weight (6.4 to 19.2 g/L dry cell weight). The process of pressurizing the 500 L bioreactor to about 10 psi to about 15 psi, actuating the valves and the pump to transfer the culture from the 500 L bioreactor to the 20,000 L is executed from a DCS (Distributed Control System) interface in the control room.

Third Stage Cultivation of Euglena gracilis

Third stage cultivation stage begins with the inoculation volume ranging between 400 L and 900 L of culture from the 500 L bioreactors and a volume of slightly concentrated fresh medium to reach approximately 3100 L to 3600 L of total volume. Typically, the starting volume of the culture is approximately 3400 L to 4100 L of culture. The third stage cultivation is grown to a wet cell weight of about 30/L to about 100 g/L.

The culture is grown in batch mode until the main carbon source reaches a lower threshold concentration. Once the lower threshold of the carbon source is reached, the concentrated carbon source, concentrated nitrogen source, and concentrated salts are fed to the culture from three separate storage vessels. The concentrated growth nutrients are fed to the culture to match the carbon source consumption of Euglena gracilis on wet cell weight basis in an exponential growth based on the rate of glucose level and the wet cell weight concentration of Euglena gracilis of the culture at the time of sampling. In certain embodiments, the lower threshold of the carbon source is from about 2 g/L to about 10 g/L, about 3 g/L to about 9 g/L, about 4 g/L to about 8 g/L, or about 5 g/L to about 7 g/L. In certain embodiments, the lower threshold of the carbon source is from about 6 g/L to about 14 g/L, about 7 g/L to about 13 g/L, about 8 g/L to about 12 g/L, or about 9 g/L to about 11 g/L.

The rate of the feeding of the concentrated media, or any combination of concentrated media ingredients to the culture is modulated to control the cell density and also the required product composition in the Euglena gracilis cells. The various growth media ingredients and the composition of the cells in the culture may be measured by online process analytical probes installed on the bubble column bioreactors. These outputs may or may not be controlled simultaneously.

The rate of the feeding of the concentrated media, or any combination of concentrated media ingredients to the culture is modulated by a linear or non-linear adaptive digital controller implemented in a supervisory control and data acquisition (SCADA) system installed either on separate personal computer or installed as a module of the distributed control system (DCS). The SCADA system can collect fermentation process data from the online analytical probes or via operator data entry on a user interface.

The SCADA executes a non-linear or linear real-time adaptive control algorithm to calculate and optimize feeding rates and feeding schedule of the concentrated media, or any combination of concentrated media ingredients to the culture of Euglena gracilis based on the online output measurement of the cell density, product composition in the cell, key media ingredients in the culture, pH, and dissolved oxygen (DO). The cell density of the culture is from about 0.1 g wet cell weight to about 150 g wet cell weight. The product composition in the cell is about 30% to about 60% carbohydrates, about 30% to about 60% protein and about 0% to about 20% oils. The key media ingredients in the culture are about 0 g/L to about 40 g/L glucose, about 0 g/L to about 5 g/L yeast extract, about 0 g/L to about 7 g/L ammonium sulfate, about 0 g/L to about 5 g/L potassium, and about 0 g/L to about 5 g/L magnesium. The pH is about 2 to about 7. The dissolved oxygen concentration is about 0 ppm to about 10 ppm. The modulation of the rate of the feeding of the concentrated media, or any combination of concentrated media ingredients to the culture of Euglena gracilis is performed through dedicated feed lines for each growth media ingredient group linked to concentrated media ingredient storage vessels by a double seat valve bank and with high resolution speed pumps on the bubble column bioreactor dedicated feed lines for each growth media ingredient. The double seat valves enable the simultaneous flow of two media ingredients feed stream through the same valve without risk of cross mixing. The valve bank can distribute the concentrated growth media ingredients to 1 or more bubble column bioreactors simultaneously and efficiently while using minimal distribution piping resources.

The dissolved oxygen (DO) in the culture media according to some embodiments described herein is about 15% to about 100%. In some embodiments, the DO value is about 15% to about 90%, about 15% to about 80%, about 15% to about 70%, about 15% to about 60%, about 15% to about 50%, about 15% to about 40%, about 15% to about 30%, about 15% to about 25%, or about 15% to about 20%. In some embodiments of the methods described herein, the specific oxygen consumption is about 10-30 mg O₂/g DCW/h, optimally 14-20 mg O₂/g DCW/h. In some embodiments of the methods described herein, the O₂ uptake rate is 0.1-40 mmol/L/h. In some embodiments of the methods described herein, the O₂ uptake rate is 0.1-20 mmol/L/h. In some embodiments of the methods described herein, the specific CO₂ evolution rate is 10-40 mg CO₂/gDCW/h, optimally 20-25 mg CO₂/gDCW/h. In some embodiments of the methods described herein, the CO₂ evolution rate is 0.1-40 mmol/L/h. In some embodiments of the methods described herein, the CO₂ evolution rate is 0.1-20 mmol/L/h.

The concentrated media ingredients are transferred from the media storage vessels varying from 1200 L to 10,000 L in capacity, to the valve bank, and then to the dedicated concentrated growth media ingredient feed lines which feed the main bioreactor.

The concentrated media nutrients are sequentially pulse-fed to the culture and are chased out of the main feeding line by chase water. The feeding of the culture in the bioreactor is based on an automatic pulse feeding schedule. The feeding schedule is a set of instructions in a pre-set DCS recipe in which the frequency and predetermined volumes per feed pulse of each concentrated media nutrient and chase water are specified. The feed schedule is the frequency of feeding based on the cell density and/or the key media ingredient levels. The timing and time of the pulse feed (or feeding event) to the culture is pre-set in the DCS recipe. The feeding schedule is a set of automated instructions in which pre-calculated volumes of concentrated growth media inputted. The pre-calculated volumes are calculated with a feed calculator based on the wet cell weight concentration. The concentrated growth media volume to fed can be delivered to the culture in the bioreactor in one single pulse or can be fed in multiple pulses. The timing of the pulses when the growth media is to be fed in multiple pulses can be set in the PLC user program interface that links the operator to the PLC. The program is integrated to the PLC.

The present disclosure includes methods for heterotrophically culturing a Euglena sp. microorganism, a Schizochytrium sp. microorganism, or a Chlorella sp. microorganism.

Accordingly, the present application includes a method of heterotrophically culturing a Euglena sp. microorganism, a Schizochytrium sp. microorganism, or a Chlorella sp. microorganism comprising: a first step of batch culturing the Euglena sp. microorganism, Schizochytrium sp. microorganism, or Chlorella sp. microorganism in a first culture medium containing one or more carbon source, one or more nitrogen source, and one or more salt; and a second step of fed-batch culturing the Euglena sp. microorganism, Schizochytrium sp. microorganism, or Chlorella sp. microorganism with a second culture medium containing one or more carbon source, one or more nitrogen source, and one or more salt.

In one embodiment, the method further comprises a third step of continuously culturing the Euglena sp. microorganism, Schizochytrium sp. microorganism, or Chlorella sp. microorganism with a third culture medium containing one or more carbon source, one or more nitrogen source, and one or more salt.

All methods described herein are applicable to Euglena sp. microorganism, Schizochytrium sp. microorganism, or Chlorella sp. microorganisms. In one embodiment, the microorganism is selected from the group consisting of Euglena gracilis, Euglena sanguinea, Euglena deses, Euglena mutabilis, Euglena acus, Euglena viridis, Euglena anabaena, Euglena geniculata, Euglena oxyuris, Euglena proxima, Euglena tripteris, Euglena chlamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabrica, Euglena granulata, Euglena obtusa, Euglena limnophila, Euglena hemichromata, Euglena variabilis, Euglena caudata, Euglena minima, Euglena communis, Euglena magnifica, Euglena terricola, Euglena velata, Euglena repulsans, Euglena clavata, Euglena lata, Euglena tuberculata, Euglena cantabrica, Euglena acusformis, Euglena ostendensis, Chlorella autotrophica, Chlorella colonials, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, Chlorella vulgaris, Schizochytrium aggregatum, Schizochytrium limacinum, Schizochytrium minutum, and combinations thereof. In another embodiment, the microorganism is Euglena gracilis.

### Media

Embodiments of the invention are directed to methods of heterotrophically culturing Euglena gracilis utilizing culture media containing a combination of one or more fermentable carbon sources, one or more non-fermentable carbon sources, one or more nitrogen sources, a combination of salts and minerals, and a combination of vitamins. Embodiments of the invention are directed to methods of heterotrophically culturing Euglena gracilis utilizing culture media containing a combination of carbon sources, nitrogen sources, and salts. Described culture media utilize all of Euglena gracilis' metabolic potential, including both aerobic and anaerobic metabolism. The combination of an oil, a sugar, an alcohol, an organic nitrogen, and an inorganic nitrogen source leads to higher conversion of input to output and faster growth of the microorganism.

In embodiments, the method of heterotrophically culturing Euglena gracilis comprises culturing the Euglena gracilis in a culture media containing one or more carbon source, one or more nitrogen source, and one or more salt.

In embodiments, the carbon source is selected from an oil, a sugar, an alcohol, carboxylic acids, ferulic acid, and combinations thereof. In embodiments the oil is an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, opium poppy, castor beans, pecan, jojoba, jatropha, macadamia, Brazil nuts, or avocado, as well as combinations thereof. In one embodiment, the oil is canola oil. The sugar may be selected from glucose, fructose, galactose, lactose, maltose, sucrose, molasses, glycerol, xylose, dextrose, honey, corn syrup, and combinations thereof. The alcohol may be selected from ethanol, methanol, isopropanol, and combinations thereof. In certain embodiments, the carbon source is glucose. The carboxylic acid may be selected from citric acid, citrate, fumaric acid, fumarate, malic acid, malate, pyruvic acid, pyruvate, succinic acid, succinate, acetic acid, acetate, lactic acid, lactate, and combinations thereof. In preferred embodiments, the carbon source is a combination of glucose and an organic acid, wherein the organic acid is selected from the group consisting of pyruvic acid, malic acid, succinic acid, lactic acid, and fumaric acid.

In embodiments, the working concentration of the carbon source is at a concentration of about 0.0005 g/L to about 0.05 g/L, about 0.005 g/L to about 0.5 g/L, about 0.05 g/L to about 1 g/L, about 0.5 g/L to about 5 g/L, about 1 g/L to about 10 g/L, about 5 g/L to about 50 g/L, about 10 g/L to about 45 g/L, about 15 g/L to about 40 g/L, about 20 g/L to about 35 g/L, about 5 g/L to about 20 g/L, about 5 g/L to about 15 g/L, about 5 g/L to about 10 g/L. In embodiments, the working concentration of the carbon source is at a concentration of about 15 g/L. In embodiments, the working concentration of the carbon source is at a concentration of about 10 g/L. In embodiments, the working concentration of the carbon source is at a concentration of about 5 g/L. In embodiments, the working concentration of the carbon source is at a concentration of about 2 g/L. In embodiments, the working concentration of the carbon source is at a concentration of about 1 g/L. In embodiments, the working concentration of the carbon source is at a concentration of about 0.5 g/L. In embodiments, the working concentration of the carbon source is at a concentration of about 0.1 g/L. In embodiments, the working concentration of the carbon source is at a concentration of about 0.05 g/L. In embodiments, the working concentration of the carbon source is at a concentration of about 0.005 g/L. In embodiments, the working concentration of the carbon source is at a concentration of about 0.0005 g/L.

In embodiments, the concentrated carbon source is at a concentration of about 55 g/L to about 500 g/L, about 60 g/L to about 450 g/L, about 65 g/L to about 400 g/L, about 70 g/L to about 350 g/L, about 75 g/L to about 300 g/L, about 80 g/L to about 250 g/L, about 95 g/L to about 200 g/L, or about 100 g/L to about 150 g/L. In embodiments, the concentrated carbon source is at a concentration of about 300 g/L.

In embodiments, the nitrogen source is selected from yeast extract, ammonium sulfate, glycine, urea, alanine, asparagine, corn steep, liver extract, lab lemco, peptone, skimmed milk, soy milk, tryptone, beef extract, tricine, plant source peptone, pea protein, brown rice protein, soybean peptone, MSG, aspartic acid, arginine, potato liquor, and combinations thereof. In certain embodiments, the nitrogen source is yeast extract. In certain embodiments, the nitrogen source is ammonium sulfate. In certain embodiments, the nitrogen source is a combination of yeast extract and ammonium sulfate.

In embodiments, the working concentration of the nitrogen source is at a concentration of about 1 g/L to about 15 g/L, about 1.5 g/L to about 12.5 g/L, about 2 g/L to about 10 g/L, about 2.5 g/L to about 8.5 g/L, about 3 g/L to about 8 g/L, about 3.5 g/L to about 7.5 g/L, about 4 g/L to about 7 g/L about 4.5 g/L to about 6.5 g/L, or about 5 g/L to about 6 g/L. In embodiments, the working concentration of the nitrogen source is at a concentration of about 10 g/L. In embodiments, the working concentration of the nitrogen source is at a concentration of about 5 g/L. In embodiments, the working concentration of the nitrogen source is at a concentration of about 2 g/L.

In embodiments, the concentrated nitrogen source is at a concentration of about 34 g/L to about 100 g/L, about 36 g/L to about 190 g/L, about 38 g/L to about 180 g/L, about 40 g/L to about 170 g/L, about 42 g/L to about 160 g/L, about 44 g/L to about 150 g/L, about 46 g/L to about 140 g/L, about 48 g/L to about 130 g/L, about 50 g/L to about 120 g/L, about 52 g/L to about 110 g/L, about 54 g/L to about 100 g/L, about 56 g/L to about 90 g/L, about 58 g/L to about 80 g/L, or about 60 g/L to about 70 g/L. In embodiments, the concentrated nitrogen source is at a concentration of about 50 g/L to about 250 g/L, about 55 g/L to about 240 g/L, about 65 g/L to about 220 g/L, about 75 g/L to about 200 g/L, about 80 g/L to about 190 g/L, about 85 g/L to about 180 g/L, about 90 g/L to about 170 g/L, about 95 g/L to about 160 g/L, about 100 g/L to about 150 g/L, about 105 g/L to about 140 g/L, about 110 g/L to about 130 g/L, or about 115 g/L to about 120 g/L. In embodiments, the concentrated nitrogen source is at a concentration of about 48 g/L. In embodiments, the concentrated nitrogen source is at a concentration of about 120 g/L.

In embodiments, the salt is selected from ammonium nitrate, sodium nitrate, monopotassium phosphate, magnesium sulfate, magnesium sulfate heptahydrate, calcium chloride, calcium chloride dihydrate, calcium sulfate, calcium sulfate dihydrate, calcium carbonate, diammonium phosphate, dipotassium phosphate, and combinations thereof. In certain embodiments, the salt is monopotassium phosphate, magnesium sulfate, calcium chloride, and combinations thereof. In preferred embodiments, the salt is calcium sulfate.

In embodiments, the working concentration of the salt source is at a concentration of about 0.01 g/L to about 0.05 g/L, about 0.01 g/L to about 5 g/L, about 0.1 g/L to about 4.5 g/L, about 1 g/L to about 4 g/L, about 1.5 g/L to about 3.5 g/L, or about 2 g/L to about 3 g/L. In embodiments, the working concentration of the salt source is at a concentration of about 0.01 g/L. In embodiments, the working concentration of the salt source is at a concentration of about 0.025 g/L. In embodiments, the working concentration of the salt source is at a concentration of about 0.05 g/L. In embodiments, the working concentration of the salt source is at a concentration of about 0.1 g/L. In embodiments, the working concentration of the salt source is at a concentration of about 1 g/L.

In embodiments, the concentrated salt source is at a concentration of about 0.5 g/L to about 50 g/L, about 1 g/L to about 45 g/L, about 1.5 g/L to about 40 g/L, about 2 g/L to about 35 g/L, about 2.5 g/L to about 30 g/L, about 3 g/L to about 25 g/L, about 3.5 g/L to about 20 g/L, about 4 g/L to about 15 g/L, about 4.5 g/L to about 10 g/L, or about 5 g/L to about 8.5 g/L. In embodiments, the concentrated salt source is at a concentration of about 1 g/L. In embodiments, the concentrated salt source is at a concentration of about 10 g/L.

In embodiments, the culture media further comprises a metal. The metal is selected from iron (III) chloride, iron (III) sulfate, ammonium ferrous sulfate, ferric ammonium sulfate, manganese chloride, manganese sulfate, zinc sulfate, cobalt chloride, sodium molybdate, zinc chloride, boric acid, copper chloride, copper sulfate, ammonium heptamolybdate, and combinations thereof.

In embodiments, the culture media further comprises a vitamin mixture. The vitamin mixture contains a combination of the following: biotin (vitamin B7), thiamine (vitamin B1), riboflavin (vitamin B2), niacin (vitamin B3), pantothenic acid (vitamin B5), Pyridoxine (vitamin B6), Cyanocobalamin (vitamin B12), vitamin C, vitamin D, folic acid, vitamin A, vitamin B12, vitamin E, vitamin K, and combinations thereof.

In embodiments, the concentrated growth medium comprises about 300 g/L to about 500 g/L glucose, about 150 g/L yeast extract, about 48 g/L to about 200 g/L ammonium sulfate, about 10 g/L to about 200 g/L potassium phosphate monobasic, about 10 g/L to about 250 g/L magnesium sulfate, and about 1 g/L to 2 g/L calcium sulfate.

In embodiments, the fresh growth medium comprises about 10 g/L to about 20 g/L glucose, about 2 g/L to about 5 g/L yeast extract, about 2 g/L to about 7 g/L ammonium sulfate, about 1 g/L to about 5 g/L potassium phosphate monobasic, about 1 g/L to about 5 g/L magnesium sulfate, and about 0.1 g/L to 0.5 g/L calcium sulfate.

In embodiments, the slightly concentrated fresh medium is a range between the concentrations of the fresh growth medium and those of the concentrated medium.

In embodiments, the pH of the culture media is about 2.5 to about 4.

Culture media (also known as growth media) is a media with components needed in order to grow or culture the cells as described herein. Feed media is a media with components that is added to a culture in order to replenish nutrients. Feed media is at a working concentration or a concentrated level of components to limit dilution of the culture. Feed media is a media with components that is added to a culture in order to replenish nutrients. Feed media is at a working concentration or a concentrated level of components to limit dilution of the culture. Spent media is a media that has been used for cell culture i.e. culture media that has a lower level of growth components in it then at the start of culturing.

Additional media can be culture media, feed media, recycled culture media, spent media, supplemented media, and combinations thereof. Culture media (also known as growth media) is a media with components needed in order to grow or culture the cells. It could also be known as growth media. Feed media is a media with components that is added to a culture in order to replenish nutrients. Feed media is at a working concentration or a concentrated level of components to limit dilution of the culture. Feed media is a media with components that is added to a culture in order to replenish nutrients. Spent media is a media that has been used for cell culture i.e. culture media that has a lower level of growth components in it then at the start of culturing.

A spent media is also determined by the content of carbohydrate in the media after being used for culturing cells. For instance, the spent media can contain total carbohydrate, individual carbohydrate (e.g., glucose), or any combination of individual carbohydrate components (e.g., glucose and maltose) that is less than about 50, 40, 30, 20, 15, 10, 8, 7, 6, 5, 4, 3, 2.5, 2, 1.5, 1, 0.5, 0.4, 0.3, 0.2, 0.1 g/L. The depletion of carbohydrate in the spent media can be expressed as a percentage of starting amount of carbohydrate at the beginning of a culture, or a culture cycle. In an embodiment, the spent media comprises total carbohydrate of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, 0.001% from amount at the beginning of culturing, or cycle of culturing. In addition to carbohydrate, carboxylic acid is another carbon that is utilized by the Euglena sp. microorganism, Schizochytrium sp. microorganism, or Chlorella sp. microorganism. Useful carboxylic acid includes citric acid, citrate, fumaric acid, fumarate, malic acid, malate, pyruvic acid, pyruvate, succinic acid, succinate, acetic acid, acetate, lactic acid, and lactate. In one embodiment, the spent media, recycled culture media, or hybrid culture media comprises carboxylic acid of less than about 20, 10, 5, 4, 3, 2, 1, 0.5, 0.4, 0.3, 0.2, or 0.1 g/L.

Recycled culture media is spent media that is used to culture cells for another passage, cycle, or for culturing cells from a different culture, lot, or strains. Recycled culture media is obtained by separating the recycled culture media from a source culture media, wherein the source culture media is in a lag phase, an exponential phase, or a stationary phase. Recycled culture media could be solely spent media, or it could be mixed with culture media (fresh growth media), and/or supplemented with one or more components that are depleted in the spent media. Recycled culture media can be obtained by separating the recycled culture media from a source culture media, wherein the source culture media is in a lag phase, an exponential phase, or a stationary phase.

A hybrid culture media (also referred to herein as hybrid media or recycled hybrid media) is a culture media that contains an amount of recycled culture media (for example, a mixture of fresh media and recycled culture media). In some embodiments, a hybrid culture media is used in accordance with methods described herein. In some embodiments, the hybrid culture media comprises about 10%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% recycled culture media. In some embodiments, the hybrid culture media comprises about 10% to about 75% recycled culture media. In some embodiments, the hybrid culture media is optionally supplemented with a carbon source. Suitable media for use in accordance with embodiments of the present invention may also be found in co-pending PCT/IB2019/055524, which was filed on Jun. 28, 2019, and published as WO/2020/003243 on Jan. 2, 2020, and is hereby incorporated by reference in its entirety.

Euglena sp. microorganisms, Schizochytrium sp. microorganisms, and/or Chlorella sp. microorganisms are cultured in liquid media to propagate biomass in accordance with the methods of the invention. In the methods of the invention, microalgal species are heterotrophically grown in a medium containing one or more carbon source, one or more nitrogen source, and/or one or more salt. Concentration or amount of media components (e.g., carbon source, nitrogen source, and/or salt(s)) described herein are contemplated for total concentration or amount of such components as well as concentration or amount of one or more individual sources of, e.g., carbon, nitrogen, and/or salt(s). For example, as described below, a carbon source may be supplied to the culture to provide a concentration of carbon source in the medium of about 0.0005 g/L to about 50 g/L. Such concentration specifically includes total carbon source concentration in the medium as well as concentration of one or more individual carbon sources in the medium (e.g., concentration of one or more organic acids).

In embodiments, the one or more carbon sources of the first culture medium, the second culture medium, and the third culture medium is each, independently of the others, selected from an oil, a sugar, an alcohol, carboxylic acids, potato liquor, ferulic acid, and combinations thereof. In embodiments the oil is an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, opium poppy, castor beans, pecan, jojoba, jatropha, macadamia, Brazil nuts, or avocado, as well as combinations thereof. In one embodiment, the oil is canola oil. The sugar may be selected from glucose, fructose, galactose, lactose, maltose, sucrose, molasses, glycerol, xylose, dextrose, honey, corn syrup, and combinations thereof. The alcohol may be selected from ethanol, methanol, isopropanol, and combinations thereof. In certain embodiments, the carbon source is glucose. The carboxylic acid may be selected from citric acid, citrate, fumaric acid, fumarate, malic acid, malate, pyruvic acid, pyruvate, succinic acid, succinate, acetic acid, acetate, lactic acid, lactate, and combinations thereof. In an embodiment, the one or more carbon sources of the first culture medium, the second culture medium, and the third culture medium is each, independently of the others, selected from glucose, dextrose, fructose, molasses, glycerol, or combinations thereof.

In embodiments, the one or more nitrogen sources of the first culture medium, the second culture medium, and the third culture medium is each, independently of the others, selected from yeast extract, ammonium sulfate, glycine, urea, alanine, asparagine, corn steep, liver extract, lab lemco, peptone, skimmed milk, soy milk, tryptone, beef extract, tricine, plant source peptone, pea protein, brown rice protein, soybean peptone, MSG, aspartic acid, arginine, potato liquor and combinations thereof. In certain embodiments, the nitrogen source is yeast extract. In certain embodiments, the nitrogen source is ammonium sulfate. In certain embodiments, the nitrogen source is a combination of yeast extract and ammonium sulfate. In an embodiment, the one or more nitrogen sources of the first culture medium, the second culture medium, and the third culture medium is each, independently of the others, selected from yeast extract, corn steep liquor, ammonium sulfate, and monosodium glutamate (MSG).

In embodiments, the one or more salts of the first culture medium, the second culture medium, and the third culture medium is each, independently of the others, selected from ammonium nitrate, sodium nitrate, monopotassium phosphate, magnesium sulfate, magnesium sulfate heptahydrate, calcium chloride, calcium chloride dihydrate, calcium sulfate, calcium sulfate dihydrate, calcium carbonate, diammonium phosphate, dipotassium phosphate, and combinations thereof. In certain embodiments, the salt is monopotassium phosphate, magnesium sulfate, calcium chloride, and combinations thereof. In an embodiment, the one or more salts of the first culture medium, the second culture medium, and the third culture medium is each, independently of the others, selected from monopotassium phosphate, magnesium sulfate, calcium chloride, calcium sulfate, or combinations thereof.

In embodiments, the concentration of the carbon source in the medium is about 0.0005 g/L to about 50 g/L, about 0.0005 g/L to about 45 g/L, about 0.0005 g/L to about 40 g/L, about 0.0005 g/L to about 35 g/L, about 0.0005 g/L to about 20 g/L, about 0.0005 g/L to about 15 g/L, about 0.0005 g/L to about 10 g/L, about 0.0005 g/L to about 8 g/L, about 0.0005 g/L to about 5 g/L, about 0.0005 g/L to about 1 g/L, about 0.0005 g/L to about 0.5 g/L, about 0.0005 g/L to about 0.05 g/L, about 0.0005 g/L to about 0.005 g/L, 0.005 g/L to about 50 g/L, about 0.005 g/L to about 45 g/L, about 0.005 g/L to about 40 g/L, about 0.005 g/L to about 35 g/L, about 0.005 g/L to about 20 g/L, about 0.005 g/L to about 15 g/L, about 0.005 g/L to about 10 g/L, about 0.005 g/L to about 8 g/L, about 0.005 g/L to about 5 g/L, about 0.005 g/L to about 1 g/L, or about 0.005 g/L to about 0.5 g/L, 0.05 g/L to about 50 g/L, about 0.05 g/L to about 45 g/L, about 0.05 g/L to about 40 g/L, about 0.05 g/L to about 35 g/L, about 0.05 g/L to about 20 g/L, about 0.05 g/L to about 15 g/L, about 0.05 g/L to about 10 g/L, about 0.05 g/L to about 8 g/L, or about 0.05 g/L to about 5 g/L. In embodiments, the concentration of the carbon source in the medium is about 0.05 g/L to about 50 g/L, about 0.05 g/L to about 45 g/L, about 0.05 g/L to about 40 g/L, about 0.05 g/L to about 35 g/L, about 0.05 g/L to about 20 g/L, about 0.05 g/L to about 15 g/L, about 0.05 g/L to about 10 g/L, about 0.05 g/L to about 8 g/L, about 0.05 g/L to about 5 g/L, about 0.05 g/L to about 1 g/L, about 0.05 g/L to about 0.5 g/L, about 1 g/L to about 50 g/L, about 1 g/L to about 45 g/L, about 1 g/L to about 40 g/L, about 1 g/L to about 35 g/L, about 1 g/L to about 20 g/L, about 1 g/L to about 15 g/L, about 1 g/L to about 10 g/L, about 1 g/L to about 8 g/L, or about 1 g/L to about 5 g/L. In embodiments, the concentration of the carbon source in the medium about 5 g/L to about 50 g/L, about 10 g/L to about 45 g/L, about 15 g/L to about 40 g/L, about 20 g/L to about 35 g/L, about 5 g/L to about 20 g/L, about 5 g/L to about 15 g/L, about 5 g/L to about 10 g/L. In embodiments, the concentration of the carbon source is at a concentration of about 15 g/L. In embodiments, the concentration of the carbon source is at a concentration of about 10 g/L. In embodiments, the concentration of the carbon source is at a concentration of about 8 g/L. In embodiments, the concentration of the carbon source is at a concentration of about 5 g/L. In embodiments, the concentration of the carbon source is at a concentration of about 4 g/L. In embodiments, the concentration of the carbon source is at a concentration of about 3 g/L. In embodiments, the concentration of the carbon source is at a concentration of about 2 g/L. In embodiments, the concentration of the carbon source is at a concentration of about 1 g/L. In embodiments, the concentration of the carbon source is at a concentration of about 0.5 g/L. In embodiments, the concentration of the carbon source is at a concentration of about 0.05 g/L.

### Culturing Process

In general, feeding cell cultures can be categorized into three culturing styles: batch, fed-batch, and continuous culture. In batch culturing, a large volume of nutrients (media) is added to a population of cells. The cells are then grown until the inputs in the media are depleted, the desired concentration of cells is reached, and/or the desired product is produced. At this point the cells are harvested and the process can be repeated. In fed-batch culturing, media is added either at a constant rate or components are added in as needed to maintain the cell population. Once it has reached a maximum volume, or product formation is reached, the majority of the cells can be harvested, and the remaining cells can then be used to start the next cycle. Fed-batch can continue until the fermenter is full or nearly full. Once full, and optionally at target density, continuous or semi-continuous culturing of the fed-batch culture can commence, the goal of which is maintaining a full, target density culture. Alternatively, all or most of the culture can be harvested, and optionally, the remaining culture can be used to commence another culture. During continuous culture, a sample of fixed volume is removed at regular time intervals to make measurements and/or harvest culture components, and an equal volume of fresh media is simultaneously or immediately or soon thereafter (e.g. within about 1, about 2, about 3, about 4, about 5, about 10, about 15, about 30, or about 60 minutes thereafter) added to the culture, thereby instantaneously enhancing nutrient concentrations and diluting cell concentration. In a continuous culture, the cells are cultured in media under conditions in which additions to and removals from the media can be made over an extended period of time. As such, nutrients, growth factors and space are not exhausted. Continuous cultures can follow batch fermentation, fed-batch fermentation, or combinations thereof, or, alternatively, can be directly inoculated.

In an embodiment, the method of heterotrophically culturing a Euglena sp. microorganism, Schizochytrium sp. microorganism, or Chlorella sp. microorganism is batch, fed-batch, or continuous. In another embodiment, the method of heterotrophically culturing a Euglena sp. microorganism, Schizochytrium sp. microorganism, or Chlorella sp. microorganism is batch. In another embodiment, the method of heterotrophically culturing a Euglena sp. microorganism, Schizochytrium sp. microorganism, or Chlorella sp. microorganism is fed-batch. In another embodiment, the method of heterotrophically culturing a Euglena sp. microorganism, Schizochytrium sp. microorganism, or Chlorella sp. microorganism is continuous.

In an embodiment, the method comprises maintaining the microorganism heterotrophically in an environment substantially free from light. In another embodiment, the method comprises maintaining the microorganism heterotrophically in an environment entirely free from light.

Growth of microorganisms in a culture undergoes different phases: lag phase, log (logarithmic) phase or exponential phase, stationary phase, and death phase. During lag phase, microorganisms are maturing and metabolically active but not actively dividing or reproducing. During log phase, microorganisms are dividing, increasing in numbers such as doubling. If growth is not limited, doubling will continue at a constant rate, so both the number of cells and the rate of population increase doubles with each consecutive time period. For this type of exponential growth, plotting the natural logarithm of cell number against time produces a straight line. The slope of this line is the specific growth rate of the microorganism, which is a measure of the number of divisions per cell per unit time. The slope of this line or the specific growth rate of the microorganism varies from 0.01 h⁻¹ to 0.04 h⁻¹ depending on the growth phase of the culture. The actual rate of this growth (i.e. the slope of the line) depends upon the growth conditions, which affect the frequency of cell division events and the probability of both daughter cells surviving. When the media is depleted of nutrients and enriched with wastes, exponential growth cannot continue. During stationary phase, growth rate and death rate are equal or similar, which is shown as horizontal linear part of the growth curve. Without wishing to be bound by theory, this may be due to growth limiting factor such as the depletion of an essential nutrient, and/or the formation of an inhibitory product such as an organic acid. At death phase the microorganism dies due to, for example, lack of nutrients, pH above or below the tolerance band for the microorganism, or other adverse conditions.

When a microorganism culture reaches stationary phase, the concentration of the microorganisms in a culture reaches saturation. Saturation is determined by a number of measurements, including optical density, wet cell weight, dry cell weight, cell numbers, and/or time.

In embodiments described herein, the culture or microorganism has a maximum specific growth rate (μmax, 1/h) that is 0.001-0.1 h⁻¹. In embodiments described herein, the culture or microorganism has a maximum specific growth rate (μmax, 1/h) that is (h⁻¹) 0.001-0.09, 0.001-0.08, 0.001-0.07, 0.001-0.06, 0.001-0.05, 0.001-0.04, 0.001-0.03, 0.001-0.02, 0.001-0.01, 0.002-0.09, 0.002-0.08, 0.002-0.07, 0.002-0.06, 0.002-0.05, 0.002-0.04, 0.002-0.03, 0.002-0.02, 0.002-0.01 h⁻¹, 0.003-0.09, 0.003-0.08, 0.003-0.07, 0.003-0.06, 0.003-0.05, 0.003-0.04, 0.003-0.03, 0.003-0.02, 0.003-0.01, 0.004-0.09, 0.004-0.08, 0.004-0.07, 0.004-0.06, 0.004-0.05, 0.004-0.04, 0.004-0.03, 0.004-0.02, 0.004-0.01, 0.005-0.09, 0.005-0.08, 0.005-0.07, 0.005-0.06, 0.005-0.05, 0.005-0.04, 0.005-0.03, 0.005-0.02, 0.005-0.0, 0.006-0.09, 0.006-0.08, 0.006-0.07, 0.006-0.06, 0.006-0.05, 0.006-0.04, 0.006-0.03, 0.006-0.02, or 0.006-0.01. In some embodiments, the culture or microorganism has a maximum specific growth rate (μmax, 1/h) that is about 0.004-0.062 h⁻¹.

In embodiments described herein, feeding is based on the consumption rate of the cells in the culture. Consumption rate is a measure of the amount of carbon source or glucose in the media, which results in a slowing of the cell growth. Consumption data shows that late cycle cells use less sugar, indicating that these cells are less metabolically active. To maximize the number of cells in the exponential growth phase, the cells are harvested at the same rate as the cell growth, allowing the exponential growth phase to be extended indefinitely.

In continuous culture, culture is removed from the vessel. The culture can be removed at lag, exponential or stationary phase. In an embodiment, culture is removed from the vessel at lag, exponential or stationary phase. In another embodiment, culture is removed from the vessel at lag phase. In another embodiment, culture is removed from the vessel at exponential phase. In another embodiment, culture is removed from the vessel at stationary phase.

In continuous culture, culture can also be removed from the vessel based on time interval. In an embodiment, the culture is removed at about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes from the beginning of the culture, or cycle of culture, or from a prior media addition.

In continuous culture, media is added immediately or soon after culture is removed from the vessel. In an embodiment, the media is added at about, or at least, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60, 120, or 180 minutes from the removal of the culture.

In continuous culture, a cycle is defined as the turnover of the tank or bioreactor. Different parameters for growth are monitored and controlled for in the tank or bioreactor. These include the temperature, pH, oxygenation level and agitation. A bioreactor or tank can be, e.g., 3 L to 20,000 L. For example, a bioreactor or tank may be 3 L-8 L, 36 L, 100 L and up to 20,000 L. Larger tanks are also possible such as 100,000 L or more. In an embodiment, the tank is at least 100 L, 1,000 L, 10,000 L, or 100,000 L. In another embodiment, the tank is up to 10,000 L, 100,000 L, 200,000 L, 500,000 L, or 1,000,000 L. A turnover is defined as the emptying of a vessel of one liquid such as a first media and the filling of the vessel by a second liquid such as a second media. With each subsequent emptying and filling that would represent another turnover. For example, a turnover of 2, turning over twice, or turns over 2 times would indicate that the tank was emptied and filled twice. During continuous culturing, there is substantially equal removal and addition of source media. One turnover in continuous culturing would be when the volume of the vessel has been removed and replenished in vessel. In an embodiment, the method is continuous culture in a tank or a bioreactor. In another embodiment, the method is continuous culture in a tank up to 10,000 L, 100,000 L, 200,000 L, 500,000 L or 1,000,000 L. In another embodiment, the method is continuous culture in a bioreactor up to 3 L, 5 L, 8 L, 10 L, 20 L, 30 L, 35 L, 36 L, 40 L, or 50 L. In another embodiment, the media turns over 1, 2, 3, or 4 times a day in a tank or a bioreactor. In another embodiment, the media turns over up to 300 times in 75 days. In another embodiment, the media turns over at least 75, 150, 225, or 300 times in 75 days. In another embodiment, the method is continuous culture in a tank or a bioreactor, and the Euglena sp. microorganism, Schizochytrium sp. microorganism, or Chlorella sp. microorganism is grown for up to about 75 days. In another embodiment, the method is continuous culture in a tank or a bioreactor, the Euglena sp. microorganism, Schizochytrium sp. microorganism, or Chlorella sp. microorganism is grown for up to about 75 days, and the media turns over 300 times. In a specific embodiment, the method is continuous culture in a tank, the Euglena sp. microorganism, Schizochytrium sp. microorganism, or Chlorella sp. microorganism is grown for up to about 75 days, the media turns over 300 times.

In fed-batch and continuous culture, media is added to the culture. The media can be added at lag, exponential and/or stationary phase. In an embodiment, media is added to the culture at lag, exponential or stationary phase. In another embodiment, media is added to the culture at lag phase. In another embodiment, media is added to the culture at exponential phase. In another embodiment, media is added to the culture at stationary phase. Suitable components of the media added to the culture are described in detail herein below.

In fed-batch and continuous culture, media can also be added to the culture based on time interval. In an embodiment, the media is added at about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes from beginning of the culture, or cycle of culture, or from a prior media removal. In another embodiment, the media is added at about, or at most 10 min, 15 min, 30 min, 45 min, 60 min, 90 min, 2 h, 3 h, 4 h, 5 h, 6 h, 7 h, or 8 h from beginning of the culture, or cycle of culture, or from a prior media removal. In another embodiment, the media is added at approximately the same rate as the culture is removed by the culture.

As discussed in the Examples below, replenishment of carboxylic acids (also referenced herein as organic acids) during fed-batch and continuous culturing of microalgae (e.g., Euglena) is demonstrated. This replenishment of TCA cycle intermediates (also referred to as anaplerotic replenishment) leads to surprising and significantly increased productivity of the microalgae culture. The use of organic acid(s) as a carbon source leads to increased conversion efficiency and increased net biomass and can lead to increased production of amino acids, paramylon, wax ester, antioxidant, and/or vitamins levels in the microalgae (e.g., Euglena).

Accordingly, also encompassed are methods of increasing one or more of conversion efficiency, net biomass, production of amino acids, production of paramylon, production of wax ester, production of antioxidant, and production of vitamins of or by microalgae (e.g., Euglena) or a culture thereof by supplementing a culture thereof with at least one organic acid.

The term “conversion efficiency” as used herein refers to a percentage of the biomass generated by the amount of solutes consumed by the microorganism in the source media used. When more biomass is generated with a fixed amount of media components, the conversion efficiency is higher. When less biomass is generated with a fixed amount of media components, the conversion efficiency is lower. As such, the higher “conversion efficiency” represents more conversion of solutes into biomass. In an embodiment, the conversion efficiency of cells in a media, optionally hybrid culture media, recycled culture media or supplemented media, is at least or about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or at 100% (weight biomass/weight solutes). In some embodiments of the disclosure, the conversion efficiency is about 15 to about 75%, about 20 to about 75%, about 25 to about 75%, about 30 to about 75%, about 35 to about 75%, about 40 to about 75%, about 45 to about 75%, about 50 to about 75%, about 55 to about 75%, about 60 to about 75%, about 70 to about 75%, about 25% to about 75%. In some embodiments, the conversion efficiency is about 30% to about 60%.

As discussed above, during fed-batch and continuous culture, media is added to the culture to replenish nutrients. In embodiments, in fed-batch and continuous culture, a carbon source is supplied to the culture to provide a concentration of carbon source in the culture medium or the feed medium of about 0.0005 g/L to about 50 g/L, about 0.0005 g/L to about 45 g/L, about 0.0005 g/L to about 40 g/L, about 0.0005 g/L to about 35 g/L, about 0.0005 g/L to about 20 g/L, about 0.0005 g/L to about 15 g/L, about 0.0005 g/L to about 10 g/L, about 0.0005 g/L to about 8 g/L, about 0.0005 g/L to about 5 g/L, about 0.0005 g/L to about 1 g/L, about 0.0005 g/L to about 0.5 g/L, about 0.0005 g/L to about 0.05 g/L, or about 0.0005 g/L to about 0.005 g/L. In embodiments, in fed-batch and continuous culture, a carbon source is supplied to the culture to provide a concentration of carbon source in the culture medium or the feed medium of about 0.005 g/L to about 50 g/L, about 0.005 g/L to about 45 g/L, about 0.005 g/L to about 40 g/L, about 0.005 g/L to about 35 g/L, about 0.005 g/L to about 20 g/L, about 0.005 g/L to about 15 g/L, about 0.005 g/L to about 10 g/L, about 0.005 g/L to about 8 g/L, about 0.005 g/L to about 5 g/L, about 0.005 g/L to about 1 g/L, or about 0.005 g/L to about 0.5 g/L. In embodiments, in fed-batch and continuous culture, a carbon source is supplied to the culture to provide a concentration of carbon source in the culture medium or the feed medium of about 0.05 g/L to about 50 g/L, about 0.05 g/L to about 45 g/L, about 0.05 g/L to about 40 g/L, about 0.05 g/L to about 35 g/L, about 0.05 g/L to about 20 g/L, about 0.05 g/L to about 15 g/L, about 0.05 g/L to about 10 g/L, about 0.05 g/L to about 8 g/L, about 0.05 g/L to about 5 g/L, about 0.05 g/L to about 1 g/L, or about 0.05 g/L to about 0.5 g/L. In embodiments, in fed-batch and continuous culture, a carbon source is supplied to the culture to provide a concentration of carbon source in the culture medium or the feed medium of about 1 g/L to about 50 g/L, about 1 g/L to about 45 g/L, about 1 g/L to about 40 g/L, about 1 g/L to about 35 g/L, about 1 g/L to about 20 g/L, about 1 g/L to about 15 g/L, about 1 g/L to about 10 g/L, about 1 g/L to about 8 g/L, about 1 g/L to about 5 g/L. In embodiments, in fed-batch and continuous culture, a carbon source is supplied to the culture to provide a concentration of carbon source in the culture medium or the feed medium of about 5 g/L to about 50 g/L, about 10 g/L to about 45 g/L, about 15 g/L to about 40 g/L, about 20 g/L to about 35 g/L, about 5 g/L to about 20 g/L, about 5 g/L to about 15 g/L, about 5 g/L to about 10 g/L. In embodiments, in fed-batch and continuous culture, a carbon source is supplied to the culture to provide a concentration of carbon source in the culture medium or the feed medium of about 15 g/L. In embodiments, in fed-batch and continuous culture, a carbon source is supplied to the culture to provide a concentration of carbon source in the culture medium or the feed medium of about 10 g/L. In embodiments, in fed-batch and continuous culture, a carbon source is supplied to the culture to provide a concentration of carbon source in the culture medium or the feed medium of about 8 g/L. In embodiments, in fed-batch and continuous culture, a carbon source is supplied to the culture to provide a concentration of carbon source in the culture medium or the feed medium of about 5 g/L. In embodiments, a carbon source is supplied to the culture to provide a concentration of carbon source in the medium of about 4 g/L. In embodiments, in fed-batch and continuous culture, a carbon source is supplied to the culture to provide a concentration of carbon source in the culture medium or the feed medium of about 3 g/L. In embodiments, in fed-batch and continuous culture, a carbon source is supplied to the culture to provide a concentration of carbon source in the culture medium or the feed medium of about 2 g/L. In embodiments, in fed-batch and continuous culture, a carbon source is supplied to the culture to provide a concentration of carbon source in the culture medium or the feed medium of about 1 g/L. In embodiments, in fed-batch and continuous culture, a carbon source is supplied to the culture to provide a concentration of carbon source in the culture medium or the feed medium of about 0.5 g/L. In embodiments, in fed-batch and continuous culture, a carbon source is supplied to the culture to provide a concentration of carbon source in the culture medium or the feed medium of about 0.05 g/L. Suitable carbon sources are described above and may be in any combination. In some embodiments, the culture of methods of embodiments of the disclosure have a specific glucose consumption rate of 30-75 mg/glc/gDCW/h, optionally 40-55 mg/glc/gDCW/h.

In embodiments, in fed-batch and continuous culture, the added (or replenished) carbon source includes one or more organic acids (e.g., citric acid, citrate, fumaric acid, fumarate, malic acid, malate, pyruvic acid, pyruvate, succinic acid, succinate, acetic acid, acetate, lactic acid, and lactate). In embodiments, in fed-batch and continuous culture, the added carbon source consists of one or more organic acids. Organic acids described herein may be in either protonated or deprotonated form.

In embodiments, the concentration of the nitrogen source in the medium is about 1 g/L to about 15 g/L, about 1.5 g/L to about 12.5 g/L, about 2 g/L to about 10 g/L, about 2.5 g/L to about 8.5 g/L, about 3 g/L to about 8 g/L, about 3.5 g/L to about 7.5 g/L, about 4 g/L to about 7 g/L about 4.5 g/L to about 6.5 g/L, or about 5 g/L to about 6 g/L. In embodiments, the concentration of the nitrogen source is at a concentration of about 10 g/L. In embodiments, the concentration of the nitrogen source is at a concentration of about 5 g/L. In embodiments, the concentration of the nitrogen source is at a concentration of about 2 g/L.

In embodiments, the concentration of the salt source in the medium is about 0.01 g/l to about 0.05 g/L, 0.01 g/l to about 0.1 g/L, about 0.01 g/L to about 5 g/L, about 0.1 g/L to about 4.5 g/L, about 1 g/L to about 4 g/L, about 1.5 g/L to about 3.5 g/L, or about 2 g/L to about 3 g/L. In embodiments, the concentration of the salt source is at a concentration of about 0.01 g/L. In embodiments, the concentration of the salt source is at a concentration of about 0.025 g/L. In embodiments, the concentration of the salt source is at a concentration of about 0.05 g/L. In embodiments, the concentration of the salt source is at a concentration of about 0.1 g/L. In embodiments, the concentration of the salt source is at a concentration of about 1 g/L.

Embodiments of the invention are directed to methods of heterotrophically culturing Euglena gracilis utilizing culture media containing a combination of one or more fermentable carbon sources, one or more non-fermentable carbon sources, one or more nitrogen sources, a combination of salts and minerals, and a combination of vitamins. Embodiments of the invention are directed to methods of heterotrophically culturing Euglena gracilis utilizing culture media containing a combination of carbon sources, nitrogen sources, and salts. Described culture media utilize all of Euglena gracilis' metabolic potential, including both aerobic and anaerobic metabolism. The combination of an oil, a sugar, an alcohol, an organic nitrogen, and an inorganic nitrogen source leads to higher conversion of input to output and faster growth of the microorganism.

In embodiments, any one or more of the first culture medium, the second culture medium, and/or the third culture medium, independently of the others, further comprises one or more of a trace metal mix and a vitamin mix.

In embodiments, the first culture medium further comprises one or more of a trace metal mix and a vitamin mix.

In embodiments, the second culture medium further comprises one or more of a trace metal mix and a vitamin mix.

In embodiments, the third culture medium further comprises one or more of a trace metal mix and a vitamin mix.

In embodiments, the trace metal mix comprises one or more of iron (III) chloride, iron (III) sulfate, ammonium ferrous sulfate, ferric ammonium sulfate, manganese chloride, manganese sulfate, zinc sulfate, cobalt chloride, sodium molybdate, zinc chloride, boric acid, copper chloride, copper sulfate, ammonium heptamolybdate, and combinations thereof.

In embodiments, the culture medium further comprises a vitamin mixture. The vitamin mixture contains biotin (vitamin B7), thiamine (vitamin B1), riboflavin (vitamin B2), niacin (vitamin B3), pantothenic acid (vitamin B5), Pyridoxine (vitamin B6), Cyanocobalamin (vitamin B12), vitamin C, vitamin D, folic acid, vitamin A, vitamin B12, vitamin E, vitamin K, and combinations thereof.

In embodiments, the vitamin mix comprises one or more of Vitamin B1, Vitamin B12, Vitamin B6, and Vitamin B7.

A person of skill in the art will recognize that the culture medium utilized in one stage of fermentation may or may not be the same as the culture medium utilized in other stages of fermentation. Thus, for example, when a first culture medium is used during a fermentation and a second culture medium is used during the fermentation, they may have the same or substantially the same formulation, or they may have different formulations. Likewise, when multiple additions of culture medium are performed during a single step of the methods of the invention, each addition may be of the same or substantially the same culture medium or a different culture medium. The descriptions of culture medium herein apply to any culture medium used during any steps or stages of the methods of the present invention.

The pH of a media affects growth of a microorganism in culture. The person skilled in the art can readily modify the pH of a growth media with organic acids, such as nitric acid, hydrochloric acid, sulphuric acid, and citric acid, or bases, such as sodium hydroxide, sodium carbonate, phosphoric acid, and sodium bicarbonate. The pH of the media is between about 2 to about 8, about 2.5 to about 5, about 2.5 to about 4, about 2.5 to about 3.5. In an embodiment, the culture media is maintained at a pH of between about 2 to about 8, optionally about 2.5 to about 5, optionally between about 2.5 to about 4, optionally between about 2 to about 4.

### Bioreactor Tank System

Disclosed embodiments further include a bioreactor tank system design for the growth of microorganisms at production scale, which utilizes a streamlined and efficient fermentation tank. The tank design includes features including but not limited to air nozzles, sparging stones (also referred to as spargers, with some sparging stones being referred to as microspargers) and tank aspect ratio customization to allow for efficient turnover of production material and creation of aerobic/anaerobic zones that facilitates the metabolism of all inputs. In some embodiments, both sparging stones and air nozzles are used to create the aerobic areas inside the tank and enough lift to mix the contents. While other materials are susceptible to damage when both nozzles and spargers are used, the physiology of Euglena is such that it is capable of surviving the higher pressure of the nozzle system. It should be understood, however, that embodiments of the bioreactor tank are not limited to cultivation of Euglena, as the tank design may be beneficial to a number of other materials. In general, it has been found that using both spargers and nozzles improves the fermentation process and helps to produce a greater output.

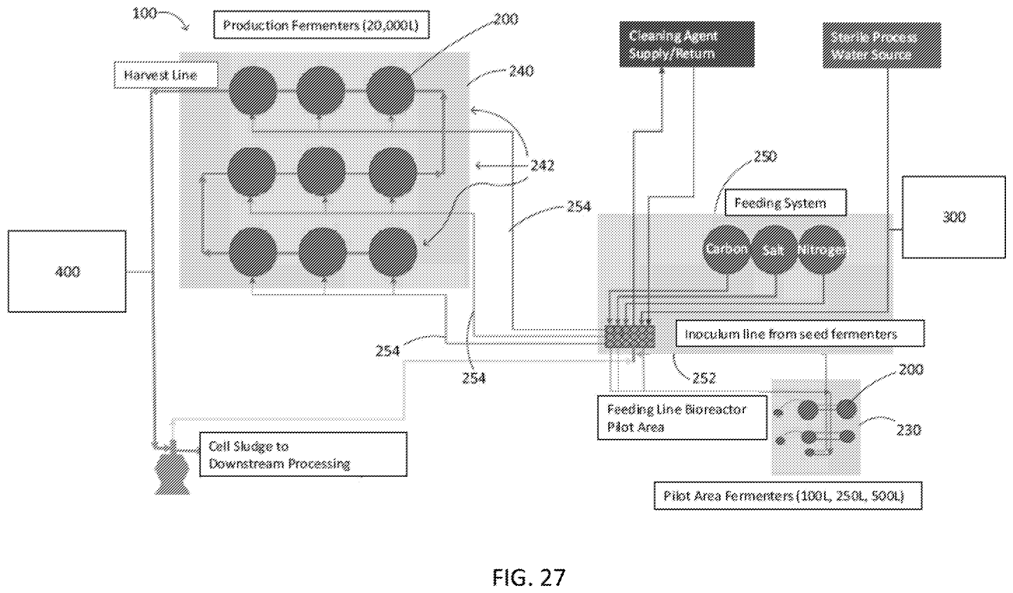

FIG. 27 is a schematic diagram of a bioreactor system 100, including a plurality of tanks 200. The system 100 is configured to produce biomass in the form of output microorganisms, such as algae. For instance, the system 100 is configured to produce Euglena on a large scale. The bioreactor system 100 may include a feeding system 250 configured to provide, for example, culture media, microorganisms, and ingredients individually to each of the bioreactor tanks 200 and/or banks of tanks. The bioreactor system 100 further includes a monitoring and control system 300 configured to provide monitoring of parameters within the bioreactor system 100 and independently control one or more features of the bioreactor system 100, such as by providing feedback control.

In an exemplary embodiment, a production system may include a plurality of tanks connected to each other. For example, the bioreactor system 100 may include pilot fermentation tanks 230 and production fermentation tanks 240. The pilot fermentation tanks 230 may include, for example, one or more relatively small tanks that help to initiate growth of a biomass. The pilot fermentation tanks 230 may include for example, a bank of three tanks, including a 100 L tank, a 250 L tank, and a 500 L tank. The feeding system 250 may include a feeding line that provides materials, such as carbon, salts, and nitrogen, to the pilot fermentation tanks 230. Line 252 is used to transfer the inoculum culture from pilot bioreactor area 230 to the production bioreactors 240.

The production fermentation tanks 240 may include groups/banks 242 of multiple tanks 200 connected in series to each other and in parallel to the feeding system 250 via a plurality of production feeding lines 254. The production fermentation tanks 240 may be of a size much larger than the pilot fermentation tanks 230. For example, the production fermentation tanks 240 may have a size of 15,000-25,000 L. For instance, the fermentation tanks 240 may be 20,000 L tanks. In other embodiments, one or more fermentation tanks 240 may have a greater size, such as 50,000 L, 200,000 L, 500,000 L or 1,000,000 L tanks.

The pilot fermentation tanks 230 may be used to bring the growth of microorganisms from a lab scale to an intermediate scale before transfer to a production fermentation tank 240 for large-scale growth and output. After the growth cycle in the larger production tanks, the culture may be transferred to a post-production area 400, which may include, for example, a surge tank and a large disk stack centrifuge for cell separation. The recovered cells may be either incubated in a secondary aerobic or anaerobic fermentation stage or disrupted for protein or beta glucan recovery. In some embodiments, the system 100 may also include smaller intermediate production tanks (not shown) of a size between tanks 230 and 240. The tanks, 230, 240 may be configured as low-pressure or high-pressure tanks. In other words, the operating pressure of the tanks 230, 240 may be selected based on desired growth parameters.

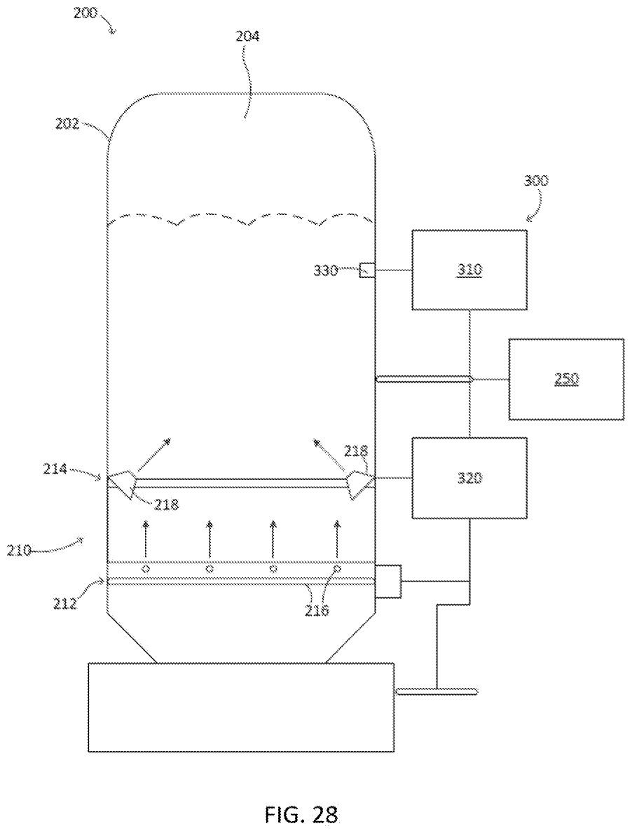

FIG. 28 is a schematic diagram of an exemplary embodiment of a bioreactor one of the tanks 200. In some embodiments, the tank 200 may be considered a bubble column bioreactor. The tank 200 includes a tank body 202 with an internal volume 204. The tank 200 is configured to receive culture media and ingredients for growing microorganisms, such as Euglena. The tank 200 further includes an air supply system 210 configured to introduce a gas into the tank 200. While the gas is described as air, it should be understood that other gasses may be introduced via the air supply system 210 components (e.g., oxygen, nitrogen, helium, etc.). The air supply system 210 may mix the culture media and microorganisms inside the internal volume 204.

In an exemplary embodiment, the air supply system 210 includes both a lower pressure supply device 212 and a higher pressure supply device 214. The lower pressure supply device 212 may be a bubbling device, such as a sparging stone 216. The higher pressure supply device 214 may be a spray nozzle 218 configured to direct a stream of gas into the internal volume 204 of the tank 200.

In some embodiments, the tank body 202 may be designed for optimal growth of the microalgae economically. While a typical aspect ratio of a bubble column bioreactors is from four to six, the tank 200 may include an aspect ratio of approximately three for the growth of microorganisms, such as Euglena. This aspect ratio is a balance between higher aspect ratio to maximize oxygen transfer and the cost incurred by installing and operating tall bubble column bioreactors. The economic benefits include lower capital costs for procuring the bioreactors and for building manufacturing areas housing the bioreactors. Taller bioreactors require more construction materials (steel beams, piping, insulation, etc.) to build the tall buildings and possible excavations in some cases. The main advantage of growing the microalgae in closed tanks is the lower risk for contamination of algal cultures by undesirable bacteria, yeasts and/or other fungi as opposed to open-system bioreactors such as exterior race ponds. In addition, the growth of the culture is not impacted by the temperature disturbances due to seasonal variations. Last but not least, shorter bioreactors are easier to clean and to preventively maintain compared to taller bioreactors.

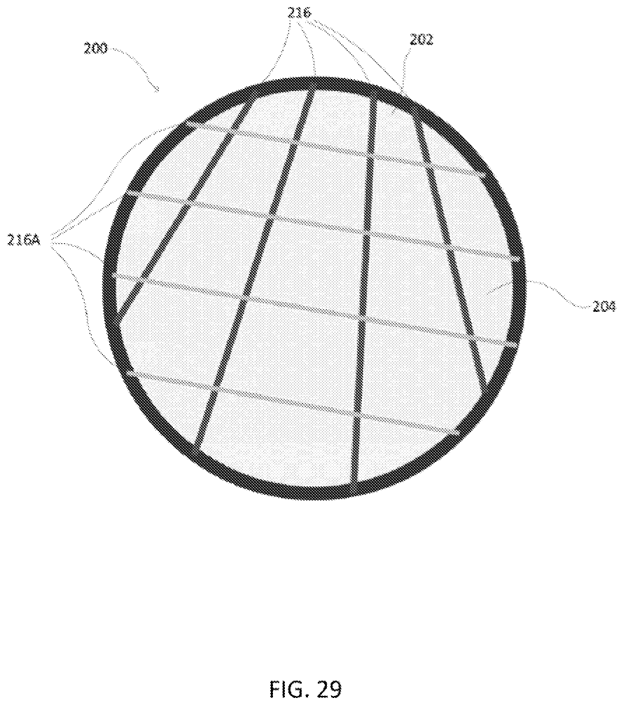

The air supply system 210, including the sparging stone 216 and the spray nozzle 218, may be an aeration system configured to create oxygen (or other gas) bubbles that oxygenate the materials inside of the internal volume 214. The aeration system may include for example, a plurality of sparging stones 216. In an exemplary embodiment, the sparging stones 216 have a small pore size, e.g., between 20-30 microns. These may be considered microspargers formed of a sintered stainless steel. The smaller pore size may provide greater bubble surface area, which has been found to promote greater oxygen transfer within the tank. The tank 200 may include a plurality of microspargers, which may be positioned in a sparger grid, as shown in FIG. 29. A first layer of spargers 216 may extend in different directions than a second layer of spargers 216A. For example, some spargers 216 may be perpendicular to other spargers 216A. The spargers 216 may include a different pore size than the spargers 216A.

The air supply system 210, including the sparging stone 216 and spray nozzle 218, may be an agitation system configured to mix the materials within the tank 200 and an aeration system configured to provide oxygen to the materials inside of the tank 200. Bulk mixing in bioreactors is typically generated by mechanical agitators which consist of impellers, a gearbox and drive (motor). According to disclosed embodiments, bulk mixing is provided by air agitation through the spray nozzles 218 in exemplary embodiments, instead of mechanical agitation (e.g., due to the fragility of the cells). However, in some embodiments, some level of mechanical agitation may be implemented in the system 100 to further promote mixing. The spray nozzles 218 may be Venturi nozzles, for example, that provide the overall turbulent bulk mixing of the vessel by generated large direction air jets to induce directional bulk mixing flow. The air jets are designed to generate shear rates that do not damage the microorganism cells. The air jet mixing may require low energy input relative to a mechanically stirred tank or vessel with a recirculation loop. In an exemplary embodiment, the spray nozzles 218 are above the sparging stones 216 and point upward at approximately a 45-degree angle. In one embodiment, the nozzles 218 are two feet above the sparging stones 216.

In some embodiments, the sparging stones 216 may also contribute to the mixing inside of the tank 200. For example, the sparging stones 216 may include some spargers that have a larger pore size than others. The larger pore size spargers may contribute to mixing while the smaller pore size spargers may focus on providing high oxygen rates. In an exemplary embodiment, a top layer of spargers 216 extend in a first direction and include a pore size of approximately 5-10 microns while a bottom layer of spargers extend in a second perpendicular direction and include a larger pore size of approximately 20-70 microns.

In some embodiments, the spray nozzles 218 may be configured to pivot to change the direction of a stream of gas. In this way, the mixing can be more precisely controlled. Each spray nozzle 218 may be configured to supply a stream of gas at a rate of about 0.1 L/minute. Each spray nozzle 218 may be positioned near the bottom of the tank 200, preferably above the spargers 216.

The feeding system 250 provides materials to the internal volume 204 of the tank 200. The feeding system 250 may include, for example, a plurality of supply tubes 210, that provide one or more ingredients for growth of microorganisms (e.g., Euglena) within the tank 200. For example, the feeding system 250 may include, for example, a water supply, algae inoculation system, sterile feed component systems(s), and/or recycled media systems. In some embodiments, the components of the feeding system may be independently controllable. In some embodiments, the feeding system 250 may include one or more independently-controllable manifolds that supply one or more tanks 200. In some embodiments, each group 242 of tanks 200 may include a controllable manifold and feed line. In other embodiments, each tank 200 (e.g., each tank 230 and/or 240) may include an associated manifold that may be independently-controllable.

The feeding system 250 allows for the simultaneous implementation of various feeding strategies and reduces fluid transfer bottlenecks. The manipulation and mixing of the concentrated media ingredients allows for the generation of concentrated media streams with tailored composition to be fed to Euglena cultures and improve the production of one targeted product over others. The feeding system 250 supports the implementation of less feed lines than tanks, as well as a discontinuous pulse feed to a continuously harvesting system. This is possible by the design and configuration of a double seat valve bank which increases the fluid transfer flexibility while reducing capital costs.

The tank 200 further includes a monitoring and control system 300, in at least some embodiments. The monitoring system 300 may include, for example, a feedback controller 310 and an input controller 320. The monitoring system 300 may also include one or more sensors 330 configured to produce a signal indicative of a performance parameter of the tank 200. The parameter may include, for example, pH, dissolved oxygen (DO), cell density, lumen level, glucose level, temperature, culture volume in the bioreactor, nitrogen levels (e.g. ammonium, glutamate), media composition, residual molecular oxygen in bioreactor exhaust gas, carbon dioxide levels in bioreactor exhaust gas, and combinations thereof. The sensor 330 may provide the signal to the feedback controller 310. The feedback controller 310 may provide the output to a user and/or to the input controller 320. The input controller 320 may receive manual or automated instructions for adjusting an input parameter of the tank 200 or feeding system 250. For example, the input controller 320 may adjust a feed rate of a material into the tank 200. In another example, the input controller 320 may adjust the air supply system 210, such as by adjusting the air pressure, the angle of the nozzles 218, or another air supply parameter. The monitoring system 300 may be also configured to maintain a temperature in the tank 200 between 20° C. to about 35° C.

Euglena's metabolism is capable of being either anaerobic or aerobic depending on which condition the microalgae is in. Based on a metabolism standpoint, for the inputs, the oil and the alcohol are metabolized under anaerobic conditions. The rest of the inputs are metabolized most efficiently under aerobic conditions. The standard for fermentation tanks is to be only aerobic in order to support growth. Disclosed embodiments provide conditions that allow for both aerobic and anaerobic zones within the tank 200. For example, the streams of air caused by the spray nozzles 218 may create zones of high mixing (e.g., around the nozzles) and zones of low mixing (e.g., areas stagnant due to the linear air stream and flow direction). The areas of high mixing may include more oxygenation than areas of low mixing. As a result, some of the areas within the tank may include Euglena in an aerobic state while other zones include Euglena in an anaerobic state. This dual-state approach has been found to promote efficient growth of Euglena. Both aerobic and anaerobic states allow the modulation of the beta-glucan content in the cells and helps achieve an optimal cell content. Aerobic and anaerobic conditions influence the cell beta-glucan and oil content. Complete aerobic conditions promote biosynthesis of beta-glucan from glucose and the conversion of oils (wax esters) to beta-glucan. Anaerobic conditions trigger the conversion of the beta-glucan to oils (wax esters). The co-existence of the aerobic and anaerobic in the vessel allows the modulation of the beta-glucan and oils (wax esters) content in the cells and therefore the culture biomass.

In embodiments described herein, media is entering the vessel at the same rate that culture is being harvested. During harvesting, the cells are separated from the media and the excess used media is then cycled back into the original vessel. Examples of harvesting techniques that may be implemented include centrifugation to harvest, disk stack, decanter, membrane dewatering step to cell separation, settling via gravity or by chemical treatment, or low shear cell separator (micro filtration). The continuous loop is sterile in order to allow the recycling of the used media back into the culture. For example, a usable portion of the harvested media may be captured and heated and/or filtered for sterilization.

Growth and harvesting processes may take place on a continuous cycle, including allowance for cycle turnover (e.g., times the tank is filled and depleted, or when the volume of the tank during continuous is removed). Consistent with disclosed embodiments, the system 100 is configured for high turnover. For example, turnover may occur up to 4 times a day when the cells have increased replication, or as low as once every 48 hours during periods of low replication. Suitable turnover is also described herein above with respect to methods of fermentation described herein.

In embodiments, the method further comprises controlling temperature, agitation, and/or air flow rate. The temperature of the fermentation is between about 20° C. to about 30° C., optionally about 28° C. Agitation can be achieved using any suitable method, including but not limited to mechanical agitation and/or aeration (for example, by use of spargers and/or nozzles within the culturing vessel). The agitation rate according to this and any other embodiment described herein is about 20 to about 120 rpm, optionally about 50 to about 180, optionally about 50 rpm, and optionally about 180 rpm, optionally about 60 to about 120 rpm, optionally about 70 rpm to about 100 rpm, optionally about 70 rpm, optionally about 100 rpm. The air flow rate in accordance with this or any other embodiment described herein is between about 0.2 to about 1.0 vvm, optionally about 0.2 vvm. In some embodiments, the temperature may remain constant throughout the steps of methods described herein. In other embodiments, the temperature may vary during or between steps of the methods described herein.

In another embodiment, the method further comprises: maintaining a pH of between about 2.0 to about 4.0 during each of the first, second, and third fermentation steps; maintaining a temperature of about 20° C. to about 30° C. during each of the first, second, and third fermentation steps; and maintaining an environment with substantially no light during each of the first, second, and third fermentation steps. Optionally, the pH is between about 2.8 to about 3.2, the dissolved oxygen is between about 1 ppm to about 2 ppm, and the temperature is between about 27° C. to about 29° C.

### General Growth Conditions

In another embodiment, the first step of batch culturing a Euglena sp. microorganism, Schizochytrium sp. microorganism, or a Chlorella sp. microorganism comprises: obtaining Euglena sp. microorganism, Schizochytrium sp. microorganism, or Chlorella sp. microorganism cells; transferring the Euglena sp. microorganism, Schizochytrium sp. microorganism, or Chlorella sp. microorganism cells to a bioreactor having a maximum culture volume; and culturing the Euglena sp. microorganism, Schizochytrium sp. microorganism, or Chlorella sp. microorganism cells until the carbon source, the nitrogen source, or both drop to the level at which cell growth is limited.

In another embodiment, the carbon source is glucose and the Euglena sp. microorganism, Schizochytrium sp. microorganism, or Chlorella sp. microorganism is cultured until the glucose level limits cell growth.

In another embodiment, the carbon source is glucose and the Euglena sp. microorganism, Schizochytrium sp. microorganism, or Chlorella sp. microorganism is cultured until the glucose level drops below 5 g/L.

In another embodiment, the second step further comprises removing culture from the bioreactor after fed-batch culturing the Euglena sp. microorganism, Schizochytrium sp. microorganism, or Chlorella sp. microorganism, and repeating the step of fed-batch culturing the Euglena sp. microorganism, Schizochytrium sp. microorganism, or Chlorella sp. microorganism one or more times.

In another embodiment, the third step of continuously culturing the Euglena sp. microorganism, Schizochytrium sp. microorganism, or Chlorella sp. microorganism comprises: frequently or continuously adding the third culture medium to the bioreactor at a feed flow rate; and frequently or continuously harvesting culture from the bioreactor at the same rate as the feed flow rate.

In certain embodiments, the feed flow rate remains constant throughout the frequent or continuous feeding. In other embodiments, the feed flow rate is variable throughout the frequent or continuous feeding. Although the feed flow rate may vary throughout the fermentation, the feed flow rate and the rate of continuous harvesting vary at substantially the same rate, so that the total volume of the cultured Euglena sp. microorganism, Schizochytrium sp. microorganism, or Chlorella sp. microorganism remains substantially the same during the frequent or continuous feeding.

In embodiments, obtaining Euglena sp. microorganism, Schizochytrium sp. microorganism, or a Chlorella sp. microorganism cells comprises culturing the microorganism.

In embodiments, culturing the Euglena sp. microorganism, Schizochytrium sp. microorganism, or Chlorella sp. microorganism cells comprises inoculating growth medium with Euglena gracilis cells at about 1×10⁵ cells/mL to about 5×10⁷ cells/mL, optionally at about 1×10⁵ cells/mL to about 1×10⁷ cells/mL, optionally at about 2×10⁵ cells/mL to about 5×10⁶ cells/mL, optionally about 2.5×10⁵ cells/mL to about 3×10⁶ cells/mL, optionally at about 1.5×10⁷ to about 2.5×10⁷.

In another embodiment, the cell density measured as gDCW/L of the cultured Euglena sp. microorganism, Schizochytrium sp. microorganism, or Chlorella sp. microorganism at the completion of the second step of feeding the batch cultured Euglena sp. microorganism, Schizochytrium sp. microorganism, or Chlorella sp. microorganism is at least 1.5, 1.6, 1.7, 1.8, 1.9, 2.0, 2.1, 2.2, or 2.5 times higher than the cell density measured as gDCW/L at the end of the first step of batch culturing the Euglena gracilis.

In another embodiment, the cell density measured as gDCW/L of the cultured Euglena sp. microorganism, Schizochytrium sp. microorganism, or Chlorella sp. microorganism at the completion of the second step of feeding the batch cultured Euglena sp. microorganism, Schizochytrium sp. microorganism, or Chlorella sp. microorganism is at least 2.0 times higher than the cell density measured as gDCW/L at the end of the first step of batch culturing the Euglena sp. microorganism, Schizochytrium sp. microorganism, or Chlorella sp. microorganism.

In another embodiment, the first step of batch culturing the Euglena sp. microorganism, Schizochytrium sp. microorganism, or Chlorella sp. microorganism is carried out for between 1 and 7 days, and the second step of feeding the batch cultured Euglena sp. microorganism, Schizochytrium sp. microorganism, or Chlorella sp. microorganism is carried out for between 1 and 7 days.

In another embodiment, the third step of continuously culturing the Euglena sp. microorganism, Schizochytrium sp. microorganism, or Chlorella sp. microorganism comprises achieving a steady state condition.

In another embodiment, the third step of continuously culturing the Euglena sp. microorganism, Schizochytrium sp. microorganism, or Chlorella sp. microorganism is carried out for between 1 and 30 days.

In another embodiment, the productivity measured as gDCW/L/h during the first step of batch culturing Euglena sp. microorganism, Schizochytrium sp. microorganism, or Chlorella sp. microorganism is between 0.1 and 0.3.

In another embodiment, the productivity measured as gDCW/L/h during the second step of feeding the batch cultured Euglena sp. microorganism, Schizochytrium sp. microorganism, or Chlorella sp. microorganism is between 0.5 and 0.8.

In another embodiment, the productivity measured as gDCW/L/h during the third step of continuously culturing the Euglena sp. microorganism, Schizochytrium sp. microorganism, or Chlorella sp. microorganism is between 0.4 and 0.9. In another embodiment, the productivity measured as gDCW/L/h during the third step of continuously culturing the Euglena sp. microorganism, Schizochytrium sp. microorganism, or Chlorella sp. microorganism is between 0.4 and 0.9, 1.5, 1.6, 1.7, 1.8, 1.9, 2.0, 2.1, 2.2, 2.5, 3.0, or 4.0. In another embodiment, the productivity measured as gDCW/L/h during the third step of continuously culturing the Euglena sp. microorganism, Schizochytrium sp. microorganism, or Chlorella sp. microorganism is at least 0.9, 1.5, 1.6, 1.7, 1.8, 1.9, 2.0, 2.1, 2.2, 2.5, 3.0, or 4.0.

In another embodiment, the total productivity measured as gDCW/L/h across the first step of batch culturing Euglena sp. microorganism, Schizochytrium sp. microorganism, or Chlorella sp. microorganism, the second step of fed-batch culturing the microorganism, and the third step of continuously culturing the Euglena sp. microorganism, Schizochytrium sp. microorganism, or Chlorella sp. microorganism is between 0.4 and 0.9. In another embodiment, the total productivity measured as gDCW/L/h across the first step of batch culturing Euglena sp. microorganism, Schizochytrium sp. microorganism, or Chlorella sp. microorganism, the second step of fed-batch culturing the microorganism, and the third step of continuously culturing the Euglena sp. microorganism, Schizochytrium sp. microorganism, or Chlorella sp. microorganism is between 0.4 and 0.9, 1.5, 1.6, 1.7, 1.8, 1.9, 2.0, 2.1, 2.2, 2.5, 3.0, or 4.0. In another embodiment, the total productivity measured as gDCW/L/h across the first step of batch culturing Euglena sp. microorganism, Schizochytrium sp. microorganism, or Chlorella sp. microorganism, the second step of fed-batch culturing the microorganism, and the third step of continuously culturing the Euglena sp. microorganism, Schizochytrium sp. microorganism, or Chlorella sp. microorganism is at least 0.9, 1.5, 1.6, 1.7, 1.8, 1.9, 2.0, 2.1, 2.2, 2.5, 3.0, or 4.0.

Harvesting of Euglena gracilis

Once the culture volume reaches 80 to 90% of the maximum working volume of the bioreactor, part of or the entire content of the bioreactor is aseptically transferred to a surge tank or a volume buffer vessel prior to separation of the Euglena gracilis cells from the spent growth media. The cultivation may also be operated in a continuous mode. That is, the cell culture is transferred continuously at a dilution rate ranging between 0.01 and 0.05 h⁻¹. The final wet cell weight or the wet cell weight at which continuous cultivation is triggered typically ranges between 30 to 60 g/L wet cell weight (6.4 to 19.2 g/L dry cell weight). In some embodiments, the final wet cell weight ranges between 5 to 250 g/L (1.6 to 80 g/L dry cell weight), between 5 to 80 g/L (1.6 to 25.6 g/L), or between 30 to 60 g/L wet cell weight (6.4 to 19.2 g/L dry cell weight). The bubble column bioreactor containing the culture of Euglena gracilis to be harvested may or may not be pressurized to increase the volumetric rate of culture out of the production bubble column bioreactor. The culture of Euglena gracilis can also be transferred out of the bubble column bioreactor to be harvested by using a positive displacement pump.

The cells of Euglena gracilis may be settled in the surge vessel by adding a concentrated acid, such as phosphoric acid, or concentrated base, such as sodium hydroxide, to adjust the pH and to induce cell flocculation which accelerates cell settling. Once the surge tank reaches a pre-specified level or volume and that the cells are sufficiently flocculated, the harvested culture is transferred from the surge tank to a large-scale disk stack centrifuge through a 2 inch transfer line equipped with a variable speed centrifugal pump at a flow rate of 50 to 60 L/min.

The cell paste or cell sludge from the centrifugation may be transferred to a secondary fermentation bubble column bioreactor or to a cell storage tank. The centrate (spent growth media) can either be recycled back directly to the production bubble column bioreactor and/or be transferred to a liquid filtration and sterilization unit. The filtered and sterilized spent growth media is stored in a pre-sterilized vessel until needed and may or may not be incorporated into new growth media batches.

The overall scale-up factor of the Euglena gracilis cultivation is 640-fold considering the combined and total capacity of all commercial scale bubble column bioreactors from a seed 250 L cultivation of Euglena gracilis. Assuming a growth cycle of 8 days, the estimated current production rate is 270 kg dry cell weight in a 24 day-cycle. That is 2.6 metric tons of Euglena gracilis (dry weight basis) per year.

Aspects of the present disclosure also include harvesting Euglena sp. microorganism, Schizochytrium sp. microorganism, or Chlorella sp. microorganism cells and/or products produced by the methods of culturing Euglena sp. microorganism, Schizochytrium sp. microorganism, or Chlorella sp. microorganism described herein. Accordingly, aspects of the present invention also relate to Euglena sp. microorganism, Schizochytrium sp. microorganism, or Chlorella sp. microorganism cells and/or products harvested according to the methods described herein and compositions including such harvested Euglena sp. microorganism, Schizochytrium sp. microorganism, or Chlorella sp. microorganism cells and/or products.

Euglena sp. microorganism, Schizochytrium sp. microorganism, or Chlorella sp. microorganism cells and/or products contemplates Euglena sp. microorganism, Schizochytrium sp. microorganism, or Chlorella sp. microorganism biomass, extracts of Euglena sp. microorganism, Schizochytrium sp. microorganism, or Chlorella sp. microorganism biomass, and both intracellular and extracellular products of Euglena sp. microorganism, Schizochytrium sp. microorganism, or Chlorella sp. microorganism fermentation. Compositions including such harvested Euglena cells and/or products include, but are not limited, to, food (i.e., any composition intended to be or expected to be ingested by animals as a source of nutrition and/or calories), food products, food additives, food supplements, cosmetics, cosmetic supplements, fibers (e.g., bioplastic), plant fertilizer, and/or biofuel. Such compositions include, but are not limited to flour (e.g., microalgal flour), oil (e.g., microalgal oil), nutraceutical compositions (e.g., supplements, vitamin supplements, protein supplements, protein powders, oils, etc.)

In some embodiments, the Euglena sp. microorganism, Schizochytrium sp. microorganism, or Chlorella sp. microorganism cells produced by the methods of culturing Euglena sp. microorganism, Schizochytrium sp. microorganism, or Chlorella sp. microorganism described herein have increased concentrations of protein in the cell as compared to Euglena sp. microorganism, Schizochytrium sp. microorganism, or Chlorella sp. microorganism cell produced by other methods of culturing. High protein biomass from algae is an advantageous material for inclusion in food products. The methods of the invention can also provide biomass that has an amount of protein as measured by % of dry cell weight selected from the group consisting of about 20% to about 60%, about 25% to about 55%, about 30% to about 50%, and about 35% to about 45%.

In embodiments, the Euglena sp. microorganism, Schizochytrium sp. microorganism, or Chlorella sp. microorganism cells produced by the methods of culturing Euglena sp. microorganism, Schizochytrium sp. microorganism, or Chlorella sp. microorganism described herein have increased concentrations of oil in the cell as compared to Euglena sp. microorganism, Schizochytrium sp. microorganism, or Chlorella sp. microorganism cells produced by other methods of culturing.

Exemplary media and components thereof, as well as exemplary culture conditions and methods, are also described in the examples set forth herein below. The following examples are provided to illustrate embodiments of the present invention but are by no means intended to limit its scope.

### EXAMPLES

### Example 1: Preparation of Seed/Batch Medium

A number of shake flask and batch fermentation experiments were conducted in order to optimize the medium composition (carbon, nitrogen, salts, trace metal and vitamins) of E. gracilis testing 49 different media compositions. Carbon sources tested include glucose (10, 15 & 20 g/L), fructose (10 & 20 g/L) and molasses (10 & 20 g/L). Nitrogen and other components tested include; yeast extract (2, 5, 10 g/L), ethanol (2, 5, 10 g/L), vegetable oil (2, 5, 10 g/L), KH₂PO₄, MgSO₄.7H₂O, CaCl₂, 2H₂O, trace metals and vitamins, and combinations thereof. Hybrid media was also tested. With the aim to increase the productivity of Euglena biomass (gDCW/L/hr) either through fed-batch or chemostat (continuous feeding and harvesting) fermentation, heterotrophic cultivation of E. gracilis was initially started by batch fermentation in order to determine the growth characteristics of E. gracilis in a particular growth medium. The composition of the growth medium used is reflected in Table 1. The composition of this growth medium was experimentally optimized at both the shake flask and bioreactor scale, and resulted in higher growth rates of E. gracilis with improved yield of targeted products (i.e., protein, oil and paramylon) as compared to other medium compositions. The composition of the vitamin mix and the trace metal mix referred to in Table 1 are described in Tables 2 and 3, respectively.

TABLE 1
Composition and Preparation of Seed/Batch Growth Medium.

| Materials | Amount | | Comments |
|---|---|---|---|
| Glucose | 15 | g | Autoclave at 121° C. |
| Yeast extract | 5 | g | for 30 min |
| (NH₄)₂SO₄ | 2 | g | |
| KH₂PO₄ | 1 | g | |
| MgSO₄•7H₂O | 1 | g | |
| CaCl₂•2H₂O | 0.1 | g | |
| Trace metal mix | 0.4 | mL | |
| (2500×) | | | |
| Vegetable oil | 2 | mL | |
| Volume up to | 1 | L DW | |
| Adjust pH to 3.2 | | | |
| Vitamin mix (2500×) | 0.4 | mL | Add after autoclaving |

TABLE 2
Composition and Preparation of Vitamin Mix (2500×)

| | | | | Amount/L of |
|---|---|---|---|---|
| | | | | seed/batch |
| Materials | Amount | | Comments | medium |
| Vitamin B₁ (Thiamine) | 25 | g | Filter (0.2 μm) | 10 mg |
| Vitamin B₁₂ (Cyanocobalamin) | 125 | mg | sterilized & | 50 μg |
| Vitamin B₆ (Pyridoxine) | 5 | mg | keep at 4° C. | 2 μg |
| Vitamin B₇ (Biotin) | 0.25 | mg | | 0.1 μg |
| Volume up to | 1 | L DW | | |

TABLE 3
Composition and Preparation of Trace Metal Mixes (2500×) and (500×)

| | Trace Metal Mix (2500×) | | | | | | |
|---|---|---|---|---|---|---|---|
| | | | Amount/L | | | | |
| | | | of | | Trace Metal Mix | | |
| | | | seed/batch | | (500×) | | |
| Materials | Amount | | medium | | Amount | | Comments |
| FeCl₃•6H₂O | 105 | g | 42 | mg | 21 | g | Filter (0.2 μm) |
| ZnSO₄•7H₂O | 220 | g | 88 | mg | 11 | g | sterilized & |
| MnCl₂•4H₂O | 200 | g | 80 | mg | 4 | g | keep at 4° C. |
| CuSO₄•5H₂O | 1.95 | g | 0.78 | mg | 0.39 | g | |
| H₃BO₃ | 1.425 | g | 0.57 | mg | 0.285 | g | |
| Na₂MoO₄•2H₂O | 10 | g | 4 | mg | 0.9 | g | |
| Na₂EDTA•2H₂O | 125 | g | 50 | mg | 25 | g | |
| 1M HCL | 50 | mL | 20 | μL | N/A | | |

| | | | | |
|---|---|---|---|---|
| Volume up to | 1 | L DW | 1 | L DW |

In the case of fed-batch fermentation, 5× concentrated seed/batch growth medium was used as the feed medium, in which the concentration of glucose was 75 g/L. 2.5 L feed medium was prepared to in order to conduct fed-batch fermentation.

In the case of chemostat fermentation, 3× concentrated seed/batch growth medium was used as feed medium, in which the concentration of glucose was 45 g/L. 8 L feed medium was prepared in order to conduct the chemostat fermentation.

### Example 2: Preparation of Seed Inoculum

The seed/batch medium described in Table 1 was used for the preparation of seed inoculum. A mother culture of E. gracilis (approximately 20 million cells/mL, 200-500 mL in 1 L shake flask) has been maintained over time. This culture is routinely (once in every 4 days) fed with 100 mL seed/batch medium. Once the volume of the mother culture reaches to 500 mL, 300 mL of the culture (cells and media) is harvested from the shake flask and the resulting culture (˜200 mL) continues to be fed in a similar fashion as described above.

A brief description of seed inoculum preparation is as follows: on Day 4, before regular feeding to the mother culture of E. gracilis, 150 mL of seed/batch medium was inoculated with 50 mL culture broth from the mother culture.

The resulting culture (˜200 mL) was cultivated at 28° C., 150 rpm for 3-4 days.

The status of culture is checked by microscopy, and it is demonstrated that actively moving elongated cells are best for inoculation.

The cell density of resulting culture was determined by automated cell counter. A seed inoculum with a cell density of approximately 15-25 million cells/mL is suitable for inoculating the bioreactor.

### Example 3: Multi-Phase Fermentation Including Batch and Fed-Batch Fermentation

Methodology

Fermentation of E. gracilis was conducted in two steps: an initial batch fermentation phase as described in below, followed by a fed-batch fermentation phase as described herein.

Batch fermentation of E. gracilis is started by aseptically transferring 200 mL seed inoculum into a 5 L bioreactor containing 2.5 L of seed/batch medium. Hence, the culture volume at the start of batch fermentation was 2.7 L. The cell density of seed inoculum should ideally be close to 15-25 million cells/mL, as cell density at the onset (‘0’ hour) of fermentation should be approximately 1-2×10⁶ cells/mL (or optical density at 600 nm (OD600 or OD₆₀₀) should be approximately 0.5-1.0, or wet cell weight (WCW) should be approximately 2-4 g/L). Batch fermentation was carried out under the following parameters: Temperature at 28° C., pH at 3.2 controlled using 1 M NaOH, agitated at 70 rpm with a vertical flat blades (2) impeller, air flow rate of 0.2 vvm and DO was not controlled in this run. During batch fermentation, 25-30 mL samples were collected once per day, at ‘0’ ‘24’ ‘48’ and ‘72’ hours. Collection ceased after 72 hours, as glucose was undetectable in the bioreactor after 3 days of batch fermentation. Cell morphology/contamination was checked by microscopy and cell growth was monitored by automated cell counter, spectrophotometer (OD600) and wet cell weight (centrifugation). After completion of batch fermentation, all wet cell biomass (WCW) was freeze dried overnight to measure dry cell weight (DCW) of biomass. All WCW values (g/L) were plotted against DCW values (g/L) in order to calculate a correlation factor i.e., 1 WCW=0.32 DCW. The glucose concentration (g/L) in fermentation broth was measured by YSI autoanalyzer. The growth properties i.e., specific glucose uptake rate (qs, gglu/gDCW/hr), yield of dry biomass on glucose (Yxs, gDCW/gglu) and maximum specific growth rate (μmax, 1/h) of E. gracilis were calculated from the data collected during batch fermentation.

During batch fermentation, 25-30 mL samples were collected once per day, at ‘0’ ‘24’ ‘48’ and ‘72’ hours. Collection ceased after 72 hours, as glucose was undetectable in the bioreactor after 3 days of batch fermentation. Cell morphology/contamination was checked by microscopy and cell growth was monitored by automated cell counter, spectrophotometer (OD₆₀₀) and wet cell weight (centrifugation). After completion of batch fermentation, all wet cell biomass (WCW) was freeze dried overnight to measure dry cell weight (DCW) of biomass. All WCW values (g/L) were plotted against DCW values (g/L) in order to calculate a correlation factor i.e., 1 WCW=0.32 DCW. The glucose concentration (g/L) in fermentation broth was measured by YSI autoanalyzer. The growth properties i.e., specific glucose uptake rate (qₛ, gglu/gDCW/hr), yield of dry biomass on glucose (Yₓₛ, gDCW/gglu) and maximum specific growth rate (μₘₐₓ, 1/h) of E. gracilis were calculated from the data collected during batch fermentation.

In the case of automated cell counting, a 10 μL sample was loaded on both sides of the reusable slide provided by the manufacturer, which was then inserted into the Countess II FL Automated Cell Counter. “Autofocusing” is automatically adjusted by the machine/device. Once this is done after 20-30 seconds, the “COUNT” button is pressed. Samples were diluted if the cell count was over 5 million cells/mL.

In case of optical density (OD) measurement at 600 nm by spectrophotometer, samples were diluted, if required, to keep OD₆₀₀ values between 0.2-0.7. DI/DW was used as a blank.

In case of WCW, 25 mL samples were transferred to 50 mL falcon tube, which was pre-weighed. Tubes were centrifuged at 5000 rpm for 10 min. The supernatant was discarded and the cell pellets were washed once with 25 mL DI/DW. The tubes were centrifuged again at same setting. The supernatant was discarded and the tubes containing the pellets were weighed.

Tubes containing wet cells (after measuring WCW) were preserved minimum overnight at −20° C. (freezer). Samples were dried overnight in a freeze dryer, and dry biomass was weighed in tubes.

Glucose concentration was determined as follows: A sample of the supernatant was taken and measured by an YSI analytical instrument (YSI 2950) in order to determine the amount of glucose in the sample. More specifically, 1.5 mL samples in an Eppendorf tube were centrifuged at 10,000 rpm for 3 min. The supernatant was collected and loaded on to the YSI machine. The instrument can detect glucose in the range from 0.05 g/L to 9 g/L. It measures the glucose in an experimental sample and compares it to standard in order to determine the amount of glucose present.

During the initial batch fermentation phase, after glucose concentration dropped to below 5 g/L (i.e., after 48-72 hours of cultivation depending on inoculum density), the fed-batch fermentation phase commenced with the addition of 5× feed medium into the bioreactor in order to maintain glucose-limited culture condition of E. gracilis. The flow rate of feed medium (F (mL/hour)), was calculated by considering a specific-substrate uptake rate (qₛ=0.05 gglu/gDCW/hr) (which was calculated using the equation

$${q}_{s} = \frac{\frac{glu 1 - glu 0}{DCW 1}}{t 1 - t 0} ,$$

the culture volume (V=L), the dry cell density (X=gDCW/L) (measured WCW are multiplied by a factor of 0.32), and the concentration of glucose in feed medium (S_{f}=75 g/L). The specific growth uptake rate remained constant, while the cell density, culture volume, and glucose concentration variables change throughout the fermentation. Thus, the feed rate varied throughout fermentation. The equation used to calculate feed flow rate is

$$F ( \frac{mL}{h} ) = W C W ( \frac{g}{L} ) \times 0.32 \times {q}_{s} \times V \times 1000 / {S}_{f} .$$

The feed medium was continuously added into the bioreactor until the culture volume reached its maximum limit. The cultivation parameters during fed-batch fermentation were the same as those used in batch fermentation. After 72 hours of feeding, when the bioreactor was nearly full, the cultivation was stopped. Fermentation broth was harvested by centrifugation and the biomass was freeze dried for determination of protein, oil and paramylon content. Total biomass concentration was measured at the end of fermentation. Protein and oil content of the biomass is determined by near-infrared spectroscopy (NIR). Paramylon (beta-1,3-glucan) is determined by beta glycan assay kit (Megazyme).

Results

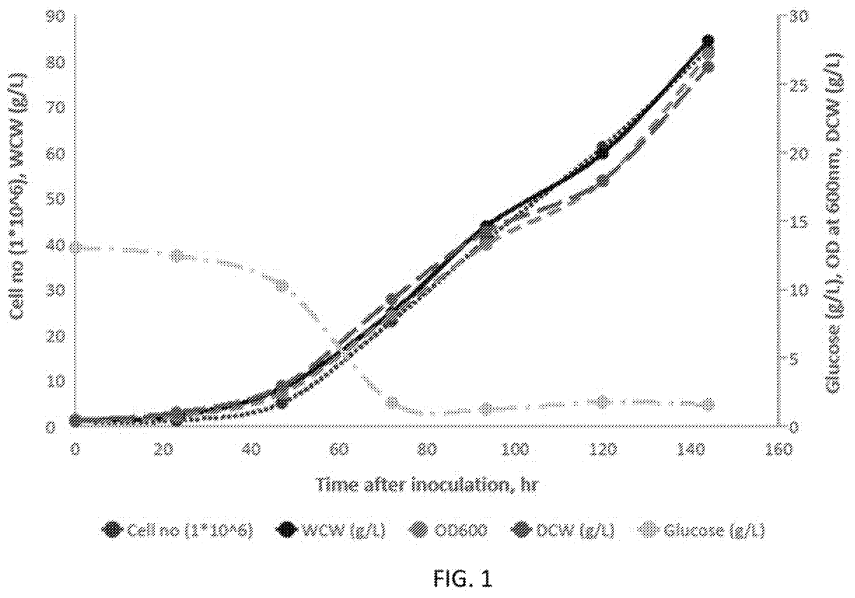

FIG. 1 and Table 4 show the E. gracilis growth characteristics of the fermentation carried out in presence of an optimized medium containing carbon (glucose), nitrogen (ammonium sulfate & yeast extract), different salts, vitamin and trace metals, as set forth in Example 1. Batch fermentation (as described in above) was conducted between hours 0-72 and fed-batch fermentation was conducted between hours 73-144. In this experiment, cell number, OD₆₀₀, and WCW at the start of cultivation were measured as 1.04×10⁶ cells/mL, 0.39 and 1.42 g/L, respectively (Table 4). The initial glucose concentration was determined to be 13.06 g/L. After 72 hours of cultivation, when the glucose concentration in the bioreactor dropped to 1.73 g/L, concentrated (5x of batch medium containing 75 g/L glucose) feed medium started to be supplied. The flow rate of feed medium (mL/hour) during fed-batch cultivation was calculated based on the equation above. After 72 hours, the feed medium was supplied at a flow rate of 13.32 mL/hour, which was increased to 26.19 mL/hour and 43.37 mL/hr at 93.5 hours and 120 hours, respectively (Table 4). The feed rates were changed due to increase in cell density and culture volume over the period of cultivation. However, the substrate uptake rate was kept constant to 0.05 gglu/gDCW/hr for calculating feeding rate.

At the end of fed-batch cultivation, the final culture volume reached approximately 4.52 L and dry cell density was 26.25 gDCW/L. In the case of this fed-batch fermentation, glucose concentration in the bioreactor were maintained below 2 g/L during feeding. The productivity during the initial batch phase was 0.129 gDCW/L/hr, however, glucose was not fully consumed, and 1.73 g/L glucose was available in the bioreactor at 72 hours. Nevertheless, productivity in this fermentation, at 0.182 gDCW/L/hr (overall) and 0.656 gDCW/L/hr (only fed-batch phase), was increased relative to the batch fermentation of Example 3. Approximately 41.5% higher productivity (overall) was obtained in this case of fed-batch fermentation than batch fermentation.

TABLE 4
Growth Characteristics of E. gracilis During Fed-Batch Fermentation.

| | Cell no. | | | | | Feeding | | | |
|---|---|---|---|---|---|---|---|---|---|
| Time | (1 × 10⁶) | | Glucose | WCW | DCW | rate | F | Reactor | |
| (hr) | cells/mL | OD₆₀₀ | (g/L) | (g/L) | (g/L) | (mL/hour) | (L/day) | Vol (L) | Phase |
| 0 | 1.04 | 0.39 | 13.07 | 1.42 | 0.45 | | | 2.65 | Batch |
| 23 | 1.28 | 0.73 | 12.47 | 2.33 | 1.00 | | | 2.60 | |
| 47 | 5.12 | 2.39 | 10.27 | 8.30 | 2.93 | | | 2.55 | |
| 72 | 23.03 | 8.12 | 1.73 | 24.98 | 9.28 | 13.32 | | 2.50 | |
| 93.5 | 40.80 | 13.30 | 1.25 | 43.85 | 14.25 | 26.19 | 0.286 | 2.78 | Fed-Batch |
| 120 | 61.18 | 17.90 | 1.76 | 59.80 | 17.95 | 43.37 | 0.694 | 3.48 | |
| 144 | 82.43 | 27.20 | 1.57 | 84.50 | 26.25 | | 1.041 | 4.52 | |

### Example 4: Multi-Phase Fermentation Including Batch, Fed-Batch, and Chemostat Fermentation

Methodology

As with the fermentation of Example 3, this multi-phase fermentation of E. gracilis was carried out in multiple steps, the first step being an initial batch fermentation phase as described in Example 3, the second step being a fed-batch fermentation phase as described in Example 3, and the third step being a chemostat fermentation phase as described herein.

During the initial batch fermentation phase, after the concentration of glucose in the bioreactor dropped to below 5 g/L, 3x feed medium was added in a fed-batch mode, at a feed rate calculated as described in Example 3, in order to increase culture volume (up to 75-80% of bioreactor's volume) with an improved cell density (15-20 gDCW/L, approximately 2-fold higher than the biomass measured at the end of batch phase). After 2-3 days of fed-batch feeding, when the volume of bioreactor reached to its maximum limit (3.75-4.0 L in case of 5 L), chemostat fermentation (i.e., continuous feeding and harvesting) was started. The feeding and harvesting rate (F (mL/hr)) during chemostat fermentation was calculated based on dilution rate (D=0.025 h⁻¹) and culture volume (V=3.75-4.0 L). The equation used to calculate feed flow rate is

$$F ( \frac{mL}{h} ) = V \times D \times 1000.$$

Maximum growth rate (μₘₐₓ) was calculated by batch fermentation. During chemostat fermentation, the dilution rate (D) should be maintained below μₘₐₓ. As μₘₐₓ was calculated to be ˜0.03-0.04 h⁻¹, D was set at 0.025 h⁻¹, in order to avoid washout. Based on the D, feed rate was calculated.

During chemostat fermentation, a steady state condition is generally achieved after 5-10 residence times (rₜ=1/D), where no accumulation of substrate, product or biomass occurred. However, steady state is only achieved at D below the maximum specific growth rate (μₘₐₓ). If D exceeds μₘₐₓ, washout of cells occurs. As it is very important to maintain the volume of bioreactor constant during chemostat fermentation, the pumps (feeding and harvesting) must be calibrated properly.

Results

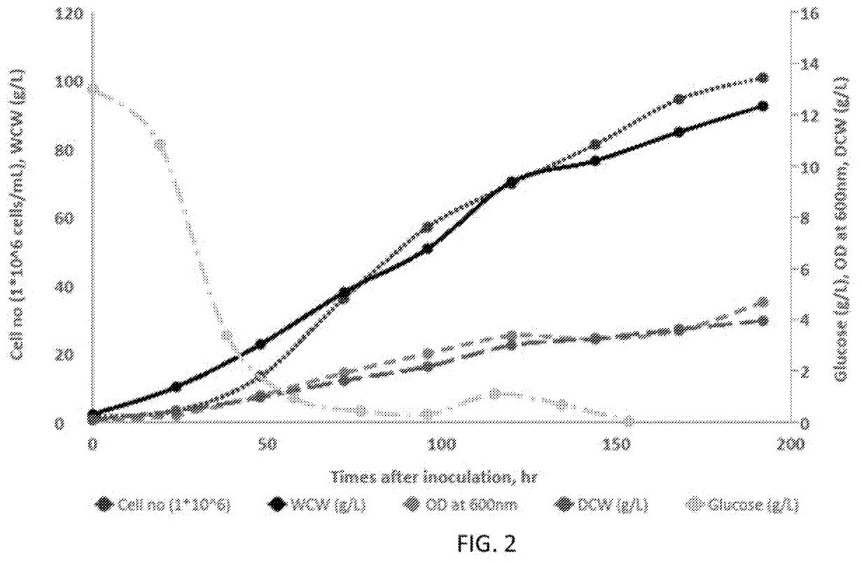

FIG. 2 shows the fermentation of E. gracilis where typical batch fermentation was conducted for 0-48 hours, fed-batch fermentation was run for 49-96 hours and chemostat fermentation was carried out for 97-192 hours, however the media was fully consumed by 171 hours. We initially started batch fermentation as per the procedure described in Example 3. In this particular experiment, cells number, OD₆₀₀ and WCW at the start of cultivation were measured 1.815×10⁶ cells/mL, 0.628 and 2.27 g/L, respectively (Table 5). The initial glucose concentration was determined to be 13.0 g/L. However, after 48 hours of cultivation, when the glucose concentration in the bioreactor dropped to 3.39 g/L, concentrated (3x of batch medium containing 45 g/L glucose) feed medium started to be supplied. The flow rate of feed medium (mL/hour) during fed-batch cultivation was calculated based on the equation

$$F ( \frac{mL}{h} ) = W C W ( \frac{g}{L} ) \times 0.32 \times {q}_{s} \times V \times 1000 / {S}_{f} .$$

After 48 hours, the feed medium was supplied at a flow rate of 17.93 mL/hour, which was increased to 36.6 mL/hour at 72 hours (Table 5). The feed rates varied due to increase in cell density and culture volume over the period of cultivation. However, the substrate uptake rate was kept constant to 0.05 gglu/gDCW/hr for calculating feeding rate.

TABLE 5
Growth Characteristics of E. gracilis During the Fermentation of
Example 4.

| | Cell no. | | | | | Feeding | | | |
|---|---|---|---|---|---|---|---|---|---|
| Time | (1 × 10⁶) | WCW | Glucose | DCW | | rates | F | Reactor | |
| (hr) | cells/mL | (g/L) | (g/L) | (g/L) | OD₆₀₀ | (mL/hour) | (L/day) | Vol (L) | Phase |
| 0 | 1.82 | 2.27 | 13.00 | 0.73 | 0.63 | | | 2.65 | Batch |
| 24 | 3.32 | 10.30 | 10.80 | 3.30 | 2.05 | | | 2.60 | |
| 48 | 13.40 | 22.80 | 3.39 | 7.28 | 7.82 | 17.93 | | 2.55 | |
| | | | | | | mL/hr | | | |
| 72 | 36.18 | 38.00 | 0.95 | 12.14 | 14.35 | 36.60 | 0.430 | 2.93 | Fed-batch |
| | | | | | | mL/hr | | | |
| 96 | 57.13 | 50.90 | 0.43 | 16.27 | 20.03 | 90.0 | 0.878 | 3.758 | |
| | | | | | | mL/hr | | | |
| 120 | 69.75 | 70.40 | 0.30 | 22.51 | 25.20 | 90.0 | 2.16 | 3.709 | Chemostat |
| | | | | | | mL/hr | | | |
| 144 | 81.25 | 76.50 | 1.10 | 24.48 | 24.30 | 90.0 | 2.16 | 3.659 | |
| | | | | | | mL/hr | | | |
| 168 | 94.50 | 84.90 | 0.68 | 27.17 | 27.00 | 90.0 | 2.16 | 3.609 | |
| | | | | | | mL/hr | | | |
| 192 | 100.75 | 92.50 | 0.03 | 29.60 | 35.10 | | 0.21 | 3.559 | |

### Example 5: Comparison of Fermentation Modes

Biomass

Generally fed-batch fermentation yields high cell density at the end of cultivation. On the other hand, chemostat cultivation yields higher productivity. However, continuous cultivation usually results in lower upstream costs due to the reduced downtime for cleaning, sterilization and setup. As reflected in Table 6, there is not much difference observed in biomass productivity between fed-batch and chemostat fermentations in this study. In case of fed-batch fermentation, the productivity was 0.656 gDCW/L/hr whereas it was 0.56-0.74 gDCW/L/hr during chemostat. However, increased dilution rates are expected to enhance cell growth rates, therefore resulting in higher cell density and/or productivity during chemostat fermentation, and thereby increasing biomass productivity. Additionally, repeated fed-batch fermentations may yield higher cell growth rates and therefore higher cell density and/or productivity during chemostat fermentation. Surprisingly, the combination of batch, fed-batch and chemostat fermentation increased starting biomass in the chemostat and, therefore, overall productivity.

TABLE 6
Productivity Throughout the Fermentation of Example 5.

| | Productivity (gDCW/L/h) | | | |
|---|---|---|---|---|
| Modes of | | Batch | Fed-batch | Continuous |
| fermentation | Overall | phase | phase | phase |
| Batch | 0.139 | N/A | N/A | N/A |
| Fed-batch | 0.182 | 0.129 | 0.656 | N/A |
| Chemostat | 0.151 | 0.152 | 0.734 | 0.56-0.74 |

Fermentation Products

Different fermentation modes also affected the production of oil and protein, as set forth in Table 7.

TABLE 7
Oil and Protein Production Throughout
the Fermentation of Example 5.

| Modes of | Oil | Protein |
|---|---|---|
| fermentation | (%) | (%) |
| Batch | 9.4 | 43.8 |
| Fed-batch | 10.0 | 27.4 |
| Chemostat | 12.5 | 37.1 |

### Example 6: Additional Fermentation Studies Using Recycled Culture Media

Monitoring of Nutrient Media During Supplementation of the Carbon Source of Recycled Media Cultivation of Euglena in a Batch Style Bioreactor

This study investigated the scale up of flask scale examples of carbon source supplemented recycled media to a 3 L bioreactor. In this experiment, nutrient monitoring is also conducted to see the rate of use glucose, ammonium, ammonium sulfate and potassium.

Methods and Materials

Seed Preparation and Generation of Recycled Media

A seed culture was prepared by inoculating 50 mL of E. gracilis cells from a mother culture into 150 mL of fresh media in 2×500 mL baffled flasks with vented caps. Cultures were kept in an incubator shaker (28° C., 120 rpm) for 3 days. At the end of incubation, all of the cells were inoculated into 3 L bioreactor containing 2.5 L of fresh media, as per Tables 1-3 (without oil). Initial cell count in the reactors was found to be approximately 1.5×10⁶ cells/mL. Glucose levels were tested using a YSI analytical instrument as described in Example 3.

Throughout the experiments, bioreactors were incubated at 28° C. with an impeller speed of 80 rpm and air flow rate of 0.4 vvm. The pH of used media was maintained at 3.2 by continual addition of 1M NaOH until the end of the fermentation. The experiments were carried out until the glucose levels in the media were quite low (i.e. ≤1 g/L). This cycle which went on for 72 h is designated as cycle 0. At the end of this cycle, all of the fermentation media (˜2.6 L) was harvested in 2×3 L sterile bottles. 200 mL of this media containing live cells was used as inoculum for starting the next cycle. Approximately 1600 mL of the media containing live biomass was centrifuged (5000 rpm, 10 minutes) in sterile centrifuge bottles to generate the recycle media (spent media) required for the next cycle (Cycle 1 or C1). Similar approach was repeatedly employed at the end of C1 to generate the spent media and seed inoculum for cycle 2 (C2) and subsequently for third cycle (C3).

For the analysis of different fermentation parameters, 30 mL of samples were taken out from both the reactors (Control and Recycled) throughout the experiments at 0, 24, 48, and if applicable, 72 hours. 5 mL of samples were used for the determination of cell count and OD (at 600 nm). Dry cell weight (DCW) was determined gravimetrically as follows. 25 mL of biomass was collected in a 50 mL preweighed centrifuged tubes. The tubes were then centrifuged at 5000 rpm for 10 min. The supernatant was separated and the tubes containing the pellet was freeze-dried to calculate the DCW. 5 mL of supernatant obtained by centrifuging 25 mL media was added into the 15 mL preweighed tubes and freeze-dried to determine the solute mass. Similarly, 5 mL was used to determine glucose, ammonium, and potassium and remaining supernatant were discarded, if any.

Cycling of Recycled Hybrid Media

Experimental Treatments

Fermentation conditions were maintained as listed above. Three cycles of growth occurred at 48 h per cycle, 200 mL of inoculum was maintained from the previous cycle to propagate the following culture (seed from Cycle 1 was used for the start of Cycle 2)

Control

The control treatment consisted of 2.5 L of fresh media and 200 mL of seed culture (inoculum), as discussed previously (without oil). Samples were taken under sterile conditions each day to monitor cell count, OD, glucose concentration, ammonium concentration, potassium concentration, dry cell weight and dry supernatant weight. Ammonium sulfate concentration was obtained by using the data of ammonium levels in the media by multiplying it by 132/36 which is the stoichiometric relationship between ammonium sulfate and ammonium based on their respective molecular weights. At the end of each cycle, lipid, protein and paramylon were measured using NIR. Biomass obtained at the end of each cycle were used as an inoculum for starting the next one.

Recycled Hybrid Media

Cell free spent media from the previous cycle was used as the cell free spent media for the subsequent cycle i.e. cell free spent media generated in the precursor cycle was used for cycle 1; cell free spent media generated in cycle 1 was used for cycle 2 and cell free spent media generated from cycle 2 was used for cycle 3. 1250 mL of cell free spent media was fed back into the bioreactor under sterile conditions. Similarly, 1250 mL of fresh media was added into the bioreactor making a total volume of 2.7 L (this included 200 mL inoculum). Samples were taken under sterile conditions each day to monitor cell count glucose concentration, ammonium concentration, potassium concentration, dry cell weight and dry supernatant weight. Ammonium sulfate concentration was obtained by using the data of ammonium levels in the media. Ammonium sulfate concentration was obtained by using the data of ammonium levels in the media by multiplying it by 132/36 which is the stoichiometric relationship between ammonium sulfate and ammonium based on their respective molecular weights. Glucose, ammonium and potassium levels were measured on a YSI 2950 instrument. Ammonium and potassium levels were determined in the same manner as glucose, with ammonium and potassium as the standards instead of glucose.

At the end of each cycle, lipid, protein and paramylon was measured using NIR. All biomass was harvested under sterile conditions, as before, cell free spent media was harvested following centrifugation and used in combination with fresh media in the subsequent cycle. Centrifugation was carried out at 5000 rpm for 10 mins in 6×250 mL sterile centrifuge bottles. At the end of centrifugation, the supernatant was collected in a 3 L sterile bottle under sterile conditions. 1250 mL of obtained supernatant was then transferred into the bioreactor containing equal volume of fresh ASAF6 media.

Data Analysis

Dried Biomass Weight

Dried biomass refers to biomass that has been freeze-dried in order to remove water molecules from the samples. The preparation of dried biomass was as described above. The skilled person can readily recognize different methods suitable for drying biomass, for example, oven drying might be used. Dried cell biomass weight over time (days) of the culture is a measure of cell growth. Cell growth could be due to more cells i.e. replication or due to compositional changes in the cell i.e. generation of carbohydrates, protein or lipids within the cell.

Dried Supernatant Weight

Supernatant was removed from the pelleted cells in the example above by decanting it from the pelleted cells and freeze drying it. This process involves freezing the cell supernatant in a 80° C. for 10 min to 12 hours before putting the sample in a Freeze dryer under vacuum. This removes the frozen water molecules. What remains is the dried solutes that were left in the media. Solutes would be the compounds i.e. components from the media as well as potential excreted materials from the cells i.e. waste products. Over time, the solutes levels will decrease as the components of the media are used, for example glucose, the major carbon source.

Determining Efficiency

Conversion efficiency is a measure of media efficiency. Conversion efficiency is defined as the amount of biomass generated divided by the total amount of solutes consumed in the media. Biomass generated is calculated by taking the total mass of biomass at the end of the cycle and subtracting the initial total biomass in the culture at the start. The total amount of solutes consumed is calculated as the total of solutes in the culture at the start minus the total solutes on the last day. Conversion efficiency is determined as follows:

Conversion efficiency=(Total biomass generated at the end of a cycle/Total solutes consumed at the end of a cycle)*100%

Total Biomass Generated

Total biomass generate per cycle is determined as follows:

Total biomass generated per cycle=total dried biomass weight at the end of a cycle−total dried biomass weight at the beginning of a cycle

Total Solutes Consumed

Total solutes consumed per cycle is determined as follows:

Total solutes consumed per cycle=Initial solutes weight at the beginning of a cycle−final solutes weight at the end of a cycle

Overall Yield

Overall Yield is the measure of how much of the inputs were converted into biomass. In this calculation the amount of biomass generated in a cycle is determined by subtracting the dried biomass at the end of each cycle in grams by the initial dried biomass weight in gram from the start of the cycle. This is then divided by the total mass of inputs used in grams i.e. all the components that are in the growth media. Dried cell weight is defined as the dried biomass weight. Overall yield is determined as follows:

Overall Yield (gDCW/gInput)=total mass of biomass (dry cell weight) generated in the cycle (gDCW)/total mass of inputs used (gInput)

Supplements Yield

Supplement Yield is calculated similarly to Overall Yield, however, instead of the total mass of inputs used, it is the total mass in the hybrid media that is from the fresh media supplementation. In this calculation the amount of biomass generated in a cycle is determined by subtracting the dried biomass at the end of each cycle in grams by the initial dried biomass weight in gram from the start of the cycle. This is then divided by the total mass of supplemented inputs used in grams i.e. all the components that are in the fresh growth media that was added. Dried cell weight is defined as the dried biomass weight. Supplement Yield is determined as follows:

Supplement Yield (gDCW/gSInput)=total mass of generated biomass (dry cell weight) in the cycle (gDCW)/total mass of inputs from the fresh growth media used in the cycle (gSInput)

Yield Based on Glucose

As glucose is the major carbon source and makes up ⅔ of the media with respect to mass, the yield in terms of glucose utilization is also reported. This is defined as the dry biomass weight generated for a cycle divided by how much glucose was used in that cycle. This is measured either as a mass (i.e. grams) or by grams per liter (concentration) of culture or growth media. Yield based on glucose (concentration) is determined as follows:

Yield based on glucose (concentration)=(concentration of cells at the end of a cycle (g/L)−concentration of cells at start of cycle (g/L))/(concentration of glucose at the start of the cycle (g/L)−concentration of glucose at the end of the cycle (g/L).

Results and Discussion

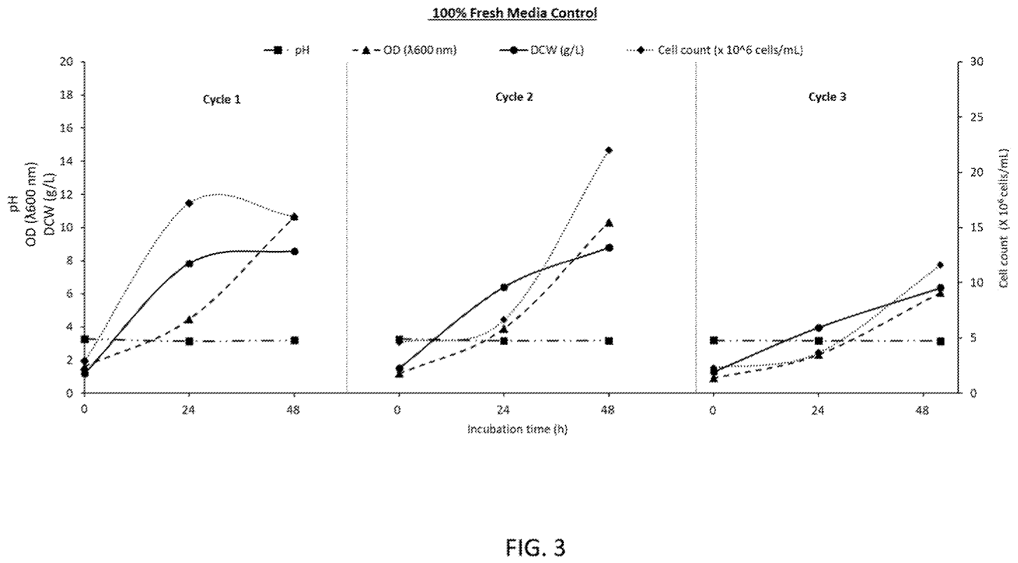

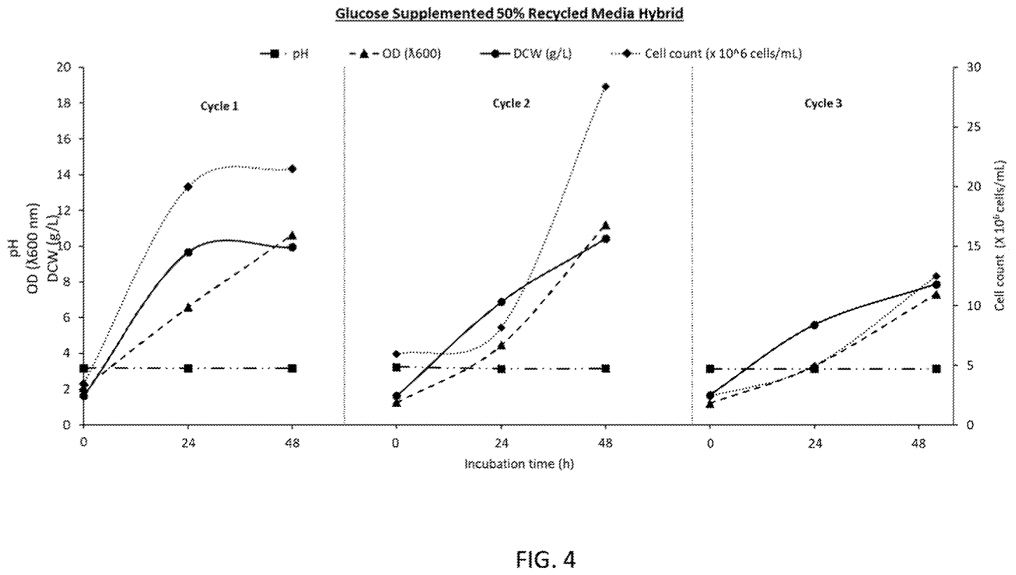

Tables 8 and 9 represent raw data for control bioreactor while Tables 10 and 11 represent raw data for the hybrid media bioreactor. Cell growth was determined by OD (600ₙₘ), cell count and dry cell weight. In general, OD, cell count, and DCW (g/L) increased over time for both recycled media bioreactor and control (FIG. 3 and FIG. 4). Control bioreactor samples were slightly higher in all cases compared to the recycled media measurements, but overall were comparable for amount of biomass generated.

TABLE 8
Control Media growth parameters OD, cell count and glucose over all
3 cycles.

| | Total Incubation | OD | Cell count | Glucose |
|---|---|---|---|---|
| Cycle | Time (h) | ( [symbol] 600 nm) | (x 10\hat{ }6 cells/mL) | (g/L) |
| Cycle 1 | 0 | 1.62 | 2.94 | 12.40 |
| | 24 | 4.47 | 17.20 | 4.55 |
| | 48 | 10.68 | 16.00 | 0.03 |
| Cycle 2 | 72 | 1.20 | 4.62 | 12.30 |
| | 96 | 3.90 | 6.62 | 6.05 |
| | 120 | 10.32 | 22.00 | 0.02 |
| Cycle 3 | 144 | 0.92 | 2.27 | 13.10 |
| | 168 | 2.33 | 3.66 | 8.65 |
| | 196 | 6.06 | 11.60 | 0.79 |

TABLE 9
Control Media growth parameters DCW (g/L), residual solute (g/L), total DCW,
total volume, and the total solute levels in the bioreactor over all 3 cycles.

| | Total | | Residual | | | |
|---|---|---|---|---|---|---|
| | Incubation | DCW | solute | Total DCW | | Total Residual |
| Cycle | Time (h) | (g/L) | (g/L) | (g) | Total vol (L) | solute (g) |
| Cycle | 0 | 1.20 | 24.20 | 3.24 | 2.70 | 65.34 |
| 1 | 24 | 7.84 | 13.60 | 20.93 | 2.67 | 36.31 |
| | 48 | 8.60 | 6.00 | 22.70 | 2.64 | 15.84 |
| Cycle | 72 | 1.52 | 25.20 | 4.10 | 2.70 | 68.04 |
| 2 | 96 | 6.40 | 16.20 | 17.09 | 2.67 | 43.25 |
| | 120 | 8.80 | 7.00 | 23.23 | 2.64 | 18.48 |
| Cycle | 144 | 1.28 | 25.20 | 3.46 | 2.70 | 68.04 |
| 3 | 168 | 3.96 | 20.00 | 10.57 | 2.67 | 53.40 |
| | 196 | 6.36 | 9.60 | 16.79 | 2.64 | 25.34 |

TABLE 10
Hybrid media growth parameters OD, cell count and glucose over all
3 cycles.

| | Total Incubation | OD | Cell count | Glucose |
|---|---|---|---|---|
| Cycle 1 | Time (h) | ( [symbol] 600 nm) | (x 10\hat{ }6 cells/mL) | (g/L) |
| Cycle 1 | 0 | 2.07 | 3.47 | 12.6 |
| | 24 | 6.58 | 20 | 3.1 |
| | 48 | 10.64 | 21.5 | 0.007 |
| Cycle 2 | 72 | 1.265 | 5.94 | 12.4 |
| | 96 | 4.46 | 8.18 | 6.31 |
| | 120 | 11.2 | 28.4 | 0.088 |
| Cycle 3 | 144 | 1.205 | 2.39 | 12.9 |
| | 168 | 3.29 | 4.96 | 8.08 |
| | 196 | 7.3 | 12.5 | 0.419 |

TABLE 11
Hybrid media growth parameters DCW (g/L),
residual solute (g/L), total DCW, total volume, and the total
solute levels in the bioreactor over all 3 cycles.

| | | | | | | Total |
|---|---|---|---|---|---|---|
| | Total | | Residual | Total | Total | Residual |
| Cycle | Incubation | DCW | solutes | DCW | volume | Solutes |
| 1 | Time (h) | (g/L) | (g/L) | (g) | (L) | (g) |
| Cycle | 0 | 1.64 | 24.2 | 4.428 | 2.7 | 65.34 |
| 1 | 24 | 9.68 | 12 | 25.8456 | 2.67 | 32.04 |
| | 48 | 9.96 | 6.8 | 26.2944 | 2.64 | 17.952 |
| Cycle | 72 | 1.64 | 25.4 | 4.428 | 2.7 | 68.58 |
| 2 | 96 | 6.88 | 15.2 | 18.3696 | 2.67 | 40.584 |
| | 120 | 10.44 | 7.8 | 27.5616 | 2.64 | 20.592 |
| Cycle | 144 | 1.68 | 25 | 4.536 | 2.7 | 67.5 |
| 3 | 168 | 5.6 | 22.2 | 14.952 | 2.67 | 59.274 |
| | 196 | 7.88 | 7.8 | 20.8032 | 2.64 | 20.592 |

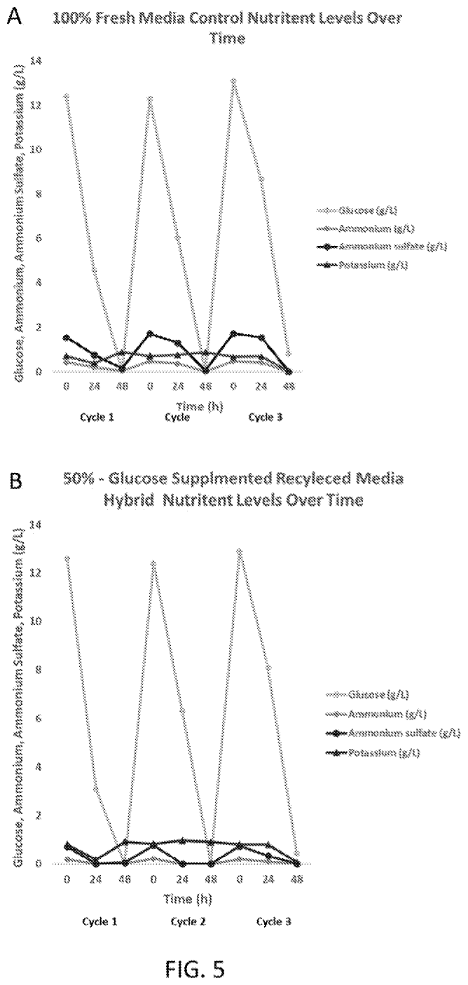

Nutrient profiles are seen in FIG. 5A and FIG. 5B for the control and hybrid media bioreactors respectfully. Glucose consumption shows a decrease in glucose with near 0 g/L levels observed by 48 hours in both the 100% fresh growth media control, as well as the glucose supplemented 50% recycled media and this trend was seen over all cycles (FIG. 5A and FIG. 5B). Ammonium concentrations decreased over time in both control and the recycled media bioreactors, and the trend was seen in all 3 cycles. The amount of ammonium in the recycled bioreactor at the start of each cycle was less than that in the fresh growth media control bioreactor. As ammonium sulfate concentration was inferred from ammonium levels, a similar trend was observed in both control and recycled media bioreactors. Ammonium sulfate concentrations in the recycled media bioreactor were approximately half that of the control, suggesting that cells are utilizing the majority of nitrogen in the growth media by the end of each cycle. Potassium levels varied in cycle 1 and 2 in the control bioreactor with lowest levels seen at 24 hours in cycle 1 and highest levels seen at 48 hours in cycle 1 and 2. However, there was a decrease in potassium levels at 48 hours in cycle 3 to near 0 g/L levels. The potassium levels in the recycled media bioreactor had similar variations with lowest level of potassium at 24 hours in cycle 1, highest levels of potassium at 48 hours in cycle 1 and at 24 hours in cycle 2. Cycle 3 again had near 0 g/L levels of potassium by 48 hours.

Conversion efficiency was calculated for all cycles for both fresh media control and glucose supplement 50% recycled media as seen in Table 12 below. The glucose supplemented 50% recycled media had a higher conversion efficiency (43%) compared to the fresh media control (36%) over all cycles. The recycled media had the highest conversion efficiency at 48% in cycle 2. If assuming fresh media was control efficiency, glucose supplemented 50% recycled media operated at a 119%% efficiency overall. This batch example compared to Example 3 and 4, where this example looked at recycling media and has shown in terms of conversion efficiency, to have a higher haver efficiency over the control, which is similar to the batch phases in Examples 3 and 4.

TABLE 12
Conversion efficiency of bioreactor scale recycled media experiment.
Cells were grown in batch style and supplemented with glucose to
bring levels to be equal to those in 100% fresh media control.

| | | 100% | 50% Recycled |
|---|---|---|---|
| Conversion | | Fresh | Media |
| Efficiency | | Media | (Glucose |
| Summary | | Control | Supplemented) |
| Cycle 1 | Total biomass (mg) | 19464 | 21866 |
| | Total Solutes consumed (mg) | 49500 | 47388 |
| | Conversion efficiency (%) | 39 | 46 |
| Cycle 2 | Total biomass (mg) | 19128 | 23134 |
| | Total Solutes consumed (mg) | 49560 | 47988 |
| | Conversion efficiency (%) | 39 | 48 |
| Cycle 3 | Total biomass (mg) | 13334 | 16267 |
| | Total Solutes consumed (mg) | 42696 | 46908 |
| | Conversion efficiency (%) | 31 | 35 |
| All cycles | Average Conversion Efficiency (%) | 36 | 43 |

Overall yield and yield based on glucose is shown below in Table 13 for 100% fresh media control and Table 14 for supplement yield and yield based on glucose for glucose supplemented 50% recycled media.

TABLE 13
summary of 100% fresh growth media overall yield and yield
based on glucose concentration.

| | | | | | | | Yield based on |
|---|---|---|---|---|---|---|---|
| | | | | | Overall | | glucose |
| | | | Volume of | Total | Yield | | (DCW(g/L))/ |
| | Age of | DCW | Culture | inputs | (gDCW/ | Glucose | Glucose |
| Cycle | culture | (g/L) | (L) | (g) | gInput) | (g/L) | (g/L)) |
| Cycle 1 | Day 0 | 1.2 | 2.7 | 65.80 | 0.296 | 12.4 | 0.60 |
| | Day 3 | 8.6 | 2.64 | | | 0.027 | |
| Cycle 2 | Day 0 | 1.52 | 2.7 | 65.80 | 0.291 | 12.3 | 0.59 |
| | Day 3 | 8.8 | 2.64 | | | 0.018 | |
| Cycle 3 | Day 0 | 1.28 | 2.7 | 65.80 | 0.203 | 13.1 | 0.41 |
| | Day 3 | 6.36 | 2.64 | | | 0.793 | |

| | | | | | |
|---|---|---|---|---|---|
| All Cycles | 23.76 g in | 197.41 | 0.263 | 36.96 | 0.54 |
| | 7.92 L | | | | |

TABLE 14
summary of glucose supplemented 50% recycled media supplement
yield and yield based on glucose concentration.

| | | | | | | | Yield based on |
|---|---|---|---|---|---|---|---|
| | | | | | Overall | | glucose |
| | | | Volume of | Total | Yield | | (DCW (g/L))/ |
| | Age of | DCW | Culture | inputs | (gDCW/ | Glucose | Glucose |
| Cycle | culture | (g/L) | (L) | (g) | gInput) | (g/L) | (g/L)) |
| Cycle 1 | Day 0 | 1.64 | 2.7 | 53.15 | 0.411 | 12.6 | 0.66 |
| | Day 3 | 9.96 | 2.64 | | | 0.007 | |
| Cycle 2 | Day 0 | 1.64 | 2.7 | 53.15 | 0.435 | 12.4 | 0.71 |
| | Day 3 | 10.44 | 2.64 | | | 0.088 | |
| Cycle 3 | Day 0 | 1.68 | 2.7 | 53.15 | 0.306 | 12.9 | 0.50 |
| | Day 3 | 7.88 | 2.64 | | | 0.419 | |

| | | | | | |
|---|---|---|---|---|---|
| All Cycles | 28.28 g in 7.92 | 159.5 | 0.384 | 37.39 | 0.62 |
| | L | | | | |

In the 100% fresh growth media the overall yield decreased from 0.296 to 0.203 as the cycled progressed, however cycle 1 and 2 were fairly similar. The same trend is seen in the yield based on glucose as cycle 1 and 2 are similar at 0.60 and 0.59 respectfully, and decreasing down to 0.41 in cycle 3. As a result the average overall yield for the control was 0.263 and for yield based on glucose 0.54.

When this is compared to the glucose supplemented 50% recycled media, the supplement yield and yield based on glucose is higher for all cycles. The supplement yield varied over the cycles with cycle 1 at 0.411, cycle 2 at 0.435, cycle 3 at the lowest of 0.306 and an average of 0.384 for all cycles. The yield based on glucose for the recycled media also varied between 0.66 for cycle 1, 0.71 for cycle 2 and 0.50 for cycle 3 with an average of 0.62 for all cycles.

Overall, the overall yield and yield based on glucose concentration was higher in the glucose supplemented 50% recycled media (hybrid media) than the 100% fresh growth media. This indicates that the amount of biomass generated based on the total amount of supplemented inputs or glucose used is higher in the 50% glucose supplemented hybrid media. Without wishing to be bound by theory, hybrid culture media has a higher yield may be due to the unique metabolism of the Euglena cell. “Waste” products that might be excreted by Euglena, such as acetic acid, lactic acid, fumaric acid, malate, pyruvate acid or succinic acid, may be able to metabolize and be useful as sources for growth. Even when taking account the amount of glucose added, the yield is still greater in the hybrid media, indicating that the Euglena cells are able to better utilized the hybrid media to generate biomass than the fresh culture media. This is seen as they generated 28 g of biomass in 7.92 L compared to the fresh media which generated 23.7 g in 7.92 L.

Based on the NIR results (Table 15, a clear trend is observed in overall biomass in each cycle and condition. The amount of paramylon (Beta-1,3-glucan) increased in the glucose supplemented 50% recycled media bioreactor compared to the 100% fresh growth media control. This is because the carbon to nitrogen ratio became higher, which supports the formulation of the carbohydrate, beta-1,3-glucan in Euglena. In terms of protein, over all cycled the percentage of protein as higher in 100% fresh growth media samples. This is expected as the carbon to nitrogen ratio in this media was lower than in recycled media condition. The lipid levels were similar between recycled and control conditions each cycle. The lowest amount of lipids was observed in the first cycle, with increased amounts in cycle 2 and cycle 3.

TABLE 15
NIR results of the biomass collected in Example 6 hybrid media and
control samples.

| | | | Lipid | Carbohydrate |
|---|---|---|---|---|
| Cycle | Sample type | Protein (%) | (%) | (%) |
| Cycle 1 | Hybrid (50% Recycled) | 30.82 | 3.25 | 65.92 |
| | 100% Fresh Control | 49.32 | 4.88 | 45.80 |
| Cycle 2 | Hybrid (50% Recycled) | 27.50 | 11.89 | 60.61 |
| | 100% Fresh Control | 49.50 | 9.10 | 41.40 |
| Cycle 3 | Hybrid (50% Recycled) | 24.80 | 8.70 | 66.50 |
| | 100% Fresh Control | 32.40 | 8.80 | 58.80 |

### Example 7: Use of Monitoring Media Components During Culturing of Euglena to Supplement Depleted Carbon Source During Continuous Culturing in a Bioreactor

In this study, glucose supplemented media was investigated in a bioreactor that undergoes all three different growth styles: batch, fed-batch, and continuous feed style. 100% fresh growth media is compared to a 50% hybrid media during batch, fed-batch and continuous culturing phases.

Methods and Materials

Preparation of Spent Media (3 Days Batch Fermentation)

Seed preparation and generation of recycled media was conducted as listed in Example 6.

To generate the recycled media for the subsequent feed style, 0.2 L of actively growing Euglena gracilis cells were inoculated into two 4 L flasks containing 3 L of media outlined in Example 6. The bioreactors were incubated at 28° C. until all glucose in the media reached near 0 g/L. Glucose consumption was measured by a YSI analytical instrument (YSI 2950) using the same method as outlined in Example 3. Ammonium and potassium levels were measured on a YSI 2950 instrument. Ammonium and potassium levels were determined in the same manner as glucose described in Example 3, with ammonium and potassium as the standards instead of glucose.

Air flow rate of at 0.4 vvm and an impeller speed of 80 rpm was maintained throughout. pH of the media was adjusted to 3.2 using 1 M NaOH solution. At the end of incubation, biomass were sterilely harvested from both the bioreactors and centrifuged at 5000 rpm for 10 mins. 5 L of the obtained cell free supernatant, also known as spent media, was sterilely transferred into 10 L flask.

Experimental Treatments

Two treatments were conducted which examined culture growth under batch, fed-batch and continuous batch fermentation conditions. The treatments were carried out in 6 L bioreactors designated as the experimental hybrid media bioreactor and the control fresh growth media bioreactor. Each of the two cultures were grown at 28° C., 0.4 vvm, impeller speed of 80 rpm and pH maintained at 3.2 by automatic addition of 1 M NaOH. Media compositions used for Example 7 are outlined below in Table 16. Initially, batch fermentation was carried out in both the reactors containing 1250 mL media A by inoculating actively growing Euglena gracilis cells. At the end of 48 h, the Hybrid Media bioreactor was fed with media D to a final working volume of 2.5 L. Similarly, in case of the control bioreactor, media C was used fill to a final volume of 2.5 L. This initiated the fed batch fermentation which was carried out for another 24 h. At the end of fed batch, the working volume was re-adjusted to 2.5 L in the bioreactors by using media D for the Hybrid Media bioreactor and C for the control bioreactor. Respective medias were fed into the systems continuously at a flow rate of 75 mL/hr. Continuous harvesting was also set up for both the tanks at similar flow rate (i.e. 75 mL/hr). Continuous fermentation was maintained for 5 days. Samples were collected every 24 h throughout the experiment under sterile conditions to measure cell count, dry cell weight (DCW), OD, solute and glucose concentration (g/L). Glucose consumption was measured by a YSI analytical instrument (YSI 2950) by the same method as outlined in Example 3.

Lipid, protein, and carbohydrate were determined using NIR at the end of a continuous cycle. Carbohydrate percentage was determined as follows:

100%−protein(%)−lipid(%)=carbohydrate percentage.

TABLE 16
Summary of Example 7 media recipes

| Compounds | Media A | Media B | Media C | Media D |
|---|---|---|---|---|
| Glucose | 15 | 22.5 | 11.25 | 1:1 ratio |
| Yeast Extract | 5 | 7.5 | 3.75 | of 50% |
| Ammonium Sulfate ((NH₄)₂SO₄) | 2 | 3 | 1.5 | spent |
| Potassium Phosphate (KH₂PO₄) | 1 | 1.5 | 0.75 | media |
| Magnesium sulfate (MgSO₄•7H₂O) | 1 | 1.5 | 0.75 | with |
| Calcium Chloride (CaCl₂•2H₂O) | 0.1 | 0.15 | 0.075 | Media B |
| Ethylenedinitrilotetraacetic acid disodium salt | 0.05 | 0.075 | 0.0375 | |
| dihydrate (Na₂EDTA•2H₂O) | | | | |
| Iron Chloride hexahydrate (FeCl₃•6H₂O) | 0.042 | 0.063 | 0.0315 | |
| Zinc sulfate heptahydrate (ZnSO₄•7H₂O) | 0.088 | 0.132 | 0.066 | |
| Manganese Chloride (MnCl₂•4H₂O) | 0.080 | 0.120 | 0.06 | |
| Copper sulfate (CuSO₄•5H₂O) | 0.78 | 1.17 | 0.585 | |
| | mg/L | mg/L | mg/L | |
| Boric Acid (H₃BO₃) | 0.57 | 0.855 | 0.4275 | |
| | mg/L | mg/L | mg/L | |
| Sodium Molybdate (Na₂MoO₄•2H₂O) | 0.004 | 0.006 | 0.002 | |
| Vitamin B₁ (Thiamine) | 0.01 | 0.015 | 0.003 | |
| Vitamin B₁₂ (Cyanocobalamin) | 0.05 | 0.075 | 0.0375 | |
| | mg/L | mg/L | mg/L | |
| Vitamin B₆ (Pyridoxine) | 0.002 | 0.003 | 0.0015 | |
| | mg/L | mg/L | mg/L | |
| Vitamin B₇ (Biotin) | 0.0001 | 0.00015 | 0.000075 | |
| | mg/L | mg/L | mg/L | |

Data Analysis

Dried Biomass weight was done as outlined in Example 6.

Dried supernatant weight was done as described in Example 6.

Further parameters that were determined in Example 6 were calculated as well.

Results and Discussion

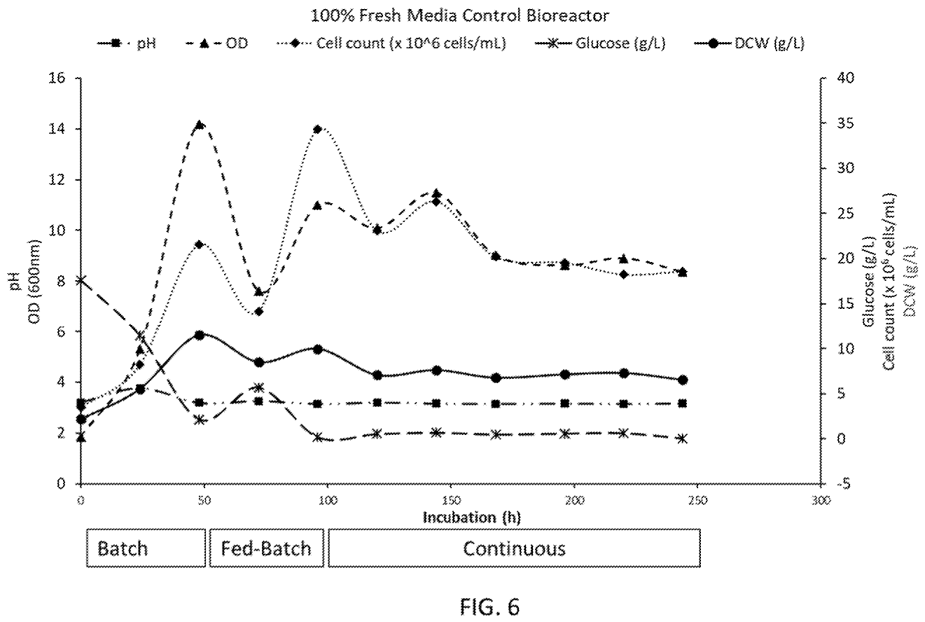

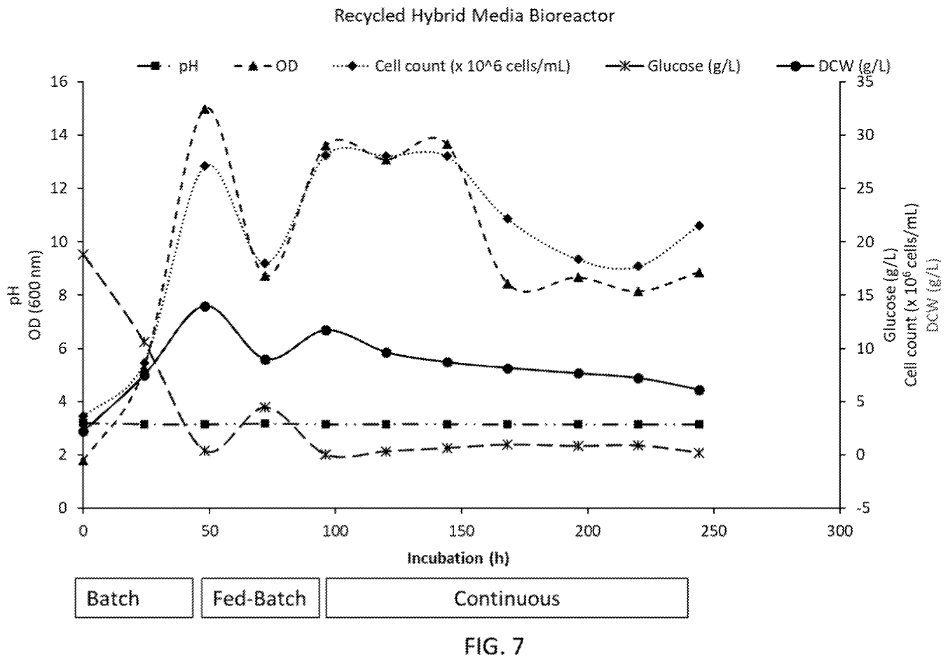

Tables 17 and 18 represents the raw data for the control bioreactor whereas Tables 19 and 20 represent the hybrid media bioreactor raw data. Cell growth was determined by OD (600 nm), cell count, dry cell weight, glucose consumption and pH (FIG. 6 and FIG. 7). For both the control and the hybrid media the batch biomass and cell count increased until the end of the cycle. This was followed by the biomass and cell count decreasing for the start of the fed batch, then increasing slightly by 24 hours. For the 100% fresh media control, the dry cell weight remained constant during the continuous phase over the 120 hours. For the hybrid media bioreactor, the dried cell weight slightly decreased over time in the continuous phase. OD and cell count for the control followed the same trend, which was varying in the start and stabilizing by the end of the phase. The hybrid media bioreactor showed more variability in the OD and cell count over the course of the continuous phase. These results suggest that the media removal and addition rate needs to be optimized for the hybrid media bioreactor, however, biomass was still generated over the course of the experiment. Glucose consumption was similar in both control and hybrid media, with decrease in batch, increased in fed batch and remained constant in the continuous phase. pH remained constant in both conditions as was controlled by 1M NaOH addition.

TABLE 17
Control Media growth parameters DCW (g/L), OD at 600 nm, cell
count, solutes, feeding rate and volume added, the reactor volume and the
harvested volume over all 3 phases of cultivation.

| | | | | Cells | | | Vol. | | |
|---|---|---|---|---|---|---|---|---|---|
| | Time | DCW | OD₆₀₀ | (1 × 10\hat{ }6 | Solutes | Feeding rate | added | Reactor | Harvested |
| Phase | (hr) | (g/L) | nm | cells/mL) | (g/L) | (mL/hr) | (L) | vol (L) | vol (L) |
| Batch | 0 | 2.16 | 1.84 | 3.45 | 35.2 | | | 1.5 | |
| | 24 | 5.52 | 5.3 | 8.2 | 26.8 | | | 1.47 | |
| | 48 | 11.5 | 14.18 | 21.6 | 20 | | | 1.44 | |
| Fed-batch | 72 | 9.96 | 11 | 34.35 | 16.4 | At 48 hr (end | 1.1 | 2.5 | |
| | | | | | | of batch | | | |
| | | | | | | phase), added | | | |
| | | | | | | 1.1 L feed | | | |
| | | | | | | medium (one | | | |
| | | | | | | shot) | | | |
| Continuous | 96 | 7.6 | 11.48 | 26.35 | 9 | 75 | 1.8 | 2.5 | 1.8 |
| | 120 | 6.76 | 9.02 | 20.2 | 5.8 | 75 | 1.8 | 2.5 | 1.8 |
| | 144 | 7.12 | 8.62 | 19.55 | 5 | 75 | 1.8 | 2.5 | 1.8 |
| | 168 | 7.28 | 8.9 | 18.25 | 5.2 | 75 | 1.8 | 2.5 | 1.8 |
| | 196 | 6.52 | 8.36 | 18.6 | 3.6 | 75 | 1.8 | 2.5 | 1.8 |

TABLE 18
Control Media growth parameters total DCW in the bioreactor, total
DCW in the harvested biomass, Total DCW, which in the continuous is
cumulative, the solutes in the reactor, the solutes in the harvested biomass
and the total solutes in the bioreactor at each stage.

| | | | Harvested | Total | Solutes | | |
|---|---|---|---|---|---|---|---|
| | Time | DCW (g) in | DCW | DCW | (g) in | Harvested | Total |
| Phase | (hr) | reactor | (g) | (g) | reactor | solutes (g) | solutes (g) |
| Batch | 0 | 3.24 | | 3.24 | 52.8 | | 52.8 |
| | 24 | 8.11 | | 8.11 | 39.4 | | 39.4 |
| | 48 | 16.53 | | 16.53 | 28.8 | | 28.8 |
| Fed-batch | 72 | 24.9 | | 24.9 | 41.0 | | 41.0 |
| Continuous | 96 | 19.0 | 13.68 | 32.68 | 22.5 | 16.2 | 38.7 |
| | 120 | 16.9 | 12.17 | 42.75 | 14.5 | 10.44 | 41.14 |
| | 144 | 17.8 | 12.82 | 56.46 | 12.5 | 9.0 | 48.14 |
| | 168 | 18.2 | 13.1 | 69.97 | 13.0 | 9.36 | 58.0 |
| | 196 | 16.3 | 11.74 | 79.8 | 9.0 | 6.48 | 60.48 |

TABLE 19
Hybrid media growth parameters DCW (g/L), OD at 600 nm,
cell count, solutes, feeding rate and volume added, the reactor volume
and the harvested volume over all 3 phases of cultivation.

| | | | | Cells | | Feeding | Vol. | | |
|---|---|---|---|---|---|---|---|---|---|
| | Time | DCW | | (1 × 10\hat{ }6 | Solutes | rate | added | Reactor | Harvested |
| Phase | (hr) | (g/L) | OD₆₀₀ | cells/mL) | (g/L) | (mL/hr) | (L) | vol (L) | vol (L) |
| Batch | 0 | 2.24 | 1.79 | 3.64 | 34.8 | | | 1.5 | |
| | 24 | 7.48 | 5.26 | 8.65 | 26.4 | | | 1.47 | |
| | 48 | 14 | 14.98 | 27.1 | 5 | | | 1.44 | |
| Fed-batch | 72 | 11.72 | 13.6 | 28.1 | 19.6 | At 48 hr | 1.1 | 2.5 | |
| | | | | | | (end of | | | |
| | | | | | | batch | | | |
| | | | | | | phase), | | | |
| | | | | | | added 1.1 L | | | |
| | | | | | | feed | | | |
| | | | | | | medium | | | |
| | | | | | | (one shot) | | | |
| Continuous | 96 | 8.72 | 13.66 | 28 | 10.6 | 75 | 1.8 | 2.5 | 1.8 |
| | 120 | 8.16 | 8.44 | 22.15 | 13 | 75 | 1.8 | 2.5 | 1.8 |
| | 144 | 7.64 | 8.66 | 18.35 | 10.8 | 75 | 1.8 | 2.5 | 1.8 |
| | 168 | 7.2 | 8.14 | 17.7 | 9.6 | 75 | 1.8 | 2.5 | 1.8 |
| | 196 | 6.12 | 8.86 | 21.5 | 8.2 | 75 | 1.8 | 2.5 | 1.8 |

TABLE 20
Hybrid Media growth parameters total DCW in the bioreactor,
total DCW in the harvested biomass, Total DCW, which in the
continuous is cumulative, the solutes in the bioreactor, the solutes in the
harvested biomass and the total solutes in the bioreactor at each stage.

| | | | | Total | | | |
|---|---|---|---|---|---|---|---|
| | Time | DCW (g) | Harvested | DCW | Solutes (g) | Harvested | Total |
| Phase | (hr) | in reactor | DCW (g) | (g) | in reactor | solutes (g) | solutes (g) |
| Batch | 0 | 3.36 | | 3.36 | 52.2 | | 52.2 |
| | 24 | 11.0 | | 11.0 | 38.81 | | 38.81 |
| | 48 | 20.16 | | 20.16 | 7.2 | | 7.2 |
| Fed-batch | 72 | 29.3 | | 29.3 | 49.0 | | 49.0 |
| Continuous | 96 | 21.8 | 15.7 | 37.5 | 26.5 | 19.08 | 45.58 |
| | 120 | 20.4 | 14.69 | 50.78 | 32.5 | 23.4 | 74.98 |
| | 144 | 19.1 | 13.75 | 63.24 | 27.0 | 19.44 | 88.92 |
| | 168 | 18.0 | 12.96 | 75.1 | 24.0 | 17.28 | 103.2 |
| | 196 | 15.3 | 11.02 | 83.41 | 20.5 | 14.76 | 114.46 |

NIR results are seen in Table 21 for Example 7. During Batch similar results are observed between control and hybrid media. During fed batch phase, there is a decrease in lipid and increase in carbohydrate in the hybrid media samples whereas there is an increase in protein in the control sample. During continuous phase, the control samples slightly increase in protein over time, while lowering in lipids and remained similar for the carbohydrates. In the hybrid media samples, a similar trend was observed as the control conditions, with a slight increase in lipids when compared to the control sample. Both conditions had less than 5% lipids, 33-41% protein and 59-64% carbohydrate. This suggests that at a continuous fermentation culture, using a hybrid media comprising of 50% recycled media gives comparable biomass composition to the 100% fresh growth media control. As well, this showed the productivity over a constant feed rate. In the next example, the feeding rate is adapted to cell growth over every 12 hour time frame.

TABLE 21
Summary of NIR results for the protein, lipid and carbohydrate
(carbs) content in Example 7 experimental conditions and time points.

| | | Control Bioreactor | | | Hybrid Media Bioreactor | | |
|---|---|---|---|---|---|---|---|
| Culture Phase | Timepoint (h) | Protein | Lipid | Carbs | Protein | Lipid | Carbs |
| End of Batch | 48 | 26.63 | 4.51 | 68.86 | 28.18 | 3.71 | 68.11 |
| Fed Batch | 0 | 28.40 | 5.65 | 65.95 | 29.16 | 0.77 | 70.06 |
| | 24 | 34.65 | 3.86 | 61.50 | 33.34 | 0.15 | 66.51 |
| Continuous | 0 | 35.30 | 4.16 | 60.54 | 32.95 | 3.33 | 63.72 |
| | 24 | 35.06 | 2.42 | 62.52 | 35.31 | 2.60 | 62.09 |
| | 48 | 39.63 | 0.33 | 60.03 | 35.80 | 3.39 | 60.82 |
| | 72 | 38.29 | 0.00 | 61.71 | 35.17 | 1.23 | 63.61 |
| | 96 | 40.59 | 0.00 | 59.41 | 39.81 | 0.95 | 59.25 |

Medium length times i.e. 3-4 days have higher conversion efficiencies compared to short cycle lengths and long cycle lengths. As well, they have a higher conversion efficiency when looking at all cycles over time i.e. at the end of all the cycles, as they on average perform better than the other cycle day lengths. Long cycle day lengths have lower conversion efficiencies as the number of cycles increases.

### Example 8: Continuous Cultivation of Euglena gracilis Using Recycled/Hybrid Medium Compared to a Control Run

1. Background

In this experiment, continuous fermentation with hybrid (recycled) media is used in a three step process of batch, fed-batch, and continuous format. These results are compared to a control experiment where fresh media is added instead of a hybrid mix.

2. Methodology:

2.1. Maintenance of Mother Culture & Preparation of Seed Inoculum:

Seed/batch/feed medium described in Tables 1-3 was used for the maintenance of the mother culture and the preparation of the seed inoculum. A mother culture of Euglena gracilis [approximately 20˜40 g/L wet cell weight (WCW), 200˜500 mL culture broth in 1 L shake flask] has been maintained in our laboratory for an extended period of time. This culture is fed thrice weekly, with 100 mL seed/batch/feed medium. Once the volume of mother culture reaches 500 mL, 300 mL of the culture broth is harvested from the shake flask and the resulting culture (˜200 mL) continues to be fed as described above.

A brief description of seed inoculum preparation is as follows.

50 mL of the mother culture of E. gracilis was inoculated to 150 mL seed/batch/feed medium in a 500 mL shake flask. 80 μL of 2500× vitamin mix is also added into the shake flask. Seed propagation was carried out at 28° C., 150 rpm for 48˜72 hours.

The status of seed inoculum is checked by microscopy, and it is demonstrated that actively moving elongated cells are the best for inoculation.

The cell density of resulting culture was determined by WCW (centrifuged 20 mL of culture broth, discarded supernatant and weighed cell pellet to determine WCW).

A seed inoculum with a cell density of approximately 20˜40 g/L WCW is used for starting a fermentation at bioreactor scale.

2.2. Continuous Fermentation:

With the aim of using recycled media for Euglena growth, while maintaining similar or better biomass yield and productivity as compared to fresh or regular medium, a continuous/chemostat fermentation was conducted in this study.

All media and concentrated stock solutions were prepared and autoclaved before beginning the experiment. A complex medium (i.e., containing glucose, yeast extract, ammonium sulfate, a range of salts, a range of vitamins, a range of trace metal salts, vegetable oil, pH adjusted to 3.2) was used throughout the entire experiment (i.e., maintenance of mother culture, seed propagation and continuous fermentation). The composition of vitamin mix and trace metal mix is described in Tables 2 and 3, respectively, and the composition of seed/batch/feed/complex medium is set forth in Table 1.

Continuous fermentation was initially started with a batch in cultivation mode. The cell density of the seed inoculum should be 20˜40 g/L WCW so that cell concentration at the onset (‘0’ hour) of fermentation is approximately OD₆₀₀ (optical density at 600 nm): 0.5˜2.0 or WCW: 2˜4 g/L. The cultivation parameters of continuous fermentation are as follows: temperature at 28° C., pH of 3.2, agitated with 300-600 rpm with a rushton turbine impeller, airflow rate of 0.4-2 vvm, and DO/pO2 at 20% using agitation and air. During fermentation, 30 mL samples were routinely collected every 12 hours. Right after sampling, the specimen were analyzed for cell morphology by microscopy, pH by pH meter, cell density by spectrophotometer (OD₆₀₀) and centrifugation of a 20 mL of culture broth (WCW), glucose concentration by YSI. Samples were further analyzed by CEDEX bioanalyzer and HPLC to determine the concentration of metabolites. Cell pellets obtained through WCW measurement were frozen at −80° C. until dry cell weight (DCW) of those samples was determined. Total solutes concentration in culture broth was also measured by freeze drying a known amount of supernatant (i.e., after removing cell pellets through centrifugation).

After running the fermentation for 36˜48 hours, the glucose concentration in the bioreactor was observed to be limiting (i.e., 0˜5 g/L). Cultivation was continued a further 2 days through fed-batch mode (i.e., feed medium was supplied into the bioreactor at a constant flow rate) before switching the cultivation to true continuous mode (i.e., continuous feeding and harvesting at a similar flow rate in order to maintain the culture volume constant). Once the glucose concentration in batch phase is close to 5 g/L, feed medium (i.e., contains 15 g/L glucose) was added at a constant flow rate without harvesting culture broth from the bioreactor. The flow rate of feed medium (F, mL/hour) was calculated using the exponential feeding formula, which is based on cell density (X=gDCW/L, measured initially as WCW that is multiplied by a factor of 0.32) and culture volume (V=L) at the end of batch phase and a constant specific glucose uptake rate (qₛ=0.07 gglu/gDCW/hr) and the concentration of glucose in feed medium (S_{f}=15 g/L). The equation used to calculate feed flow rate in fed-batch phase is as follows:

$$F ( m L / h ) = \frac{{q}_{s} . X . V}{{s}_{f}} \times 1000$$

In order to prepare the hybrid medium used in continuous fermentation, to start the continuous fermentation 1.5 L of fermentation broth were harvested and centrifuged aseptically to recover recycled medium. Once recovered, the media is aseptically mixed (1:1) with fresh medium (2500× vitamin mix was not added). The hybrid media was then added into the bioreactor at a certain dilution rate to start continuous fermentation. In this experiment, a dilution rate (D) of 0.02 h⁻¹ was set to start feeding, which is lower than the critical dilution (D_{crit})/maximum specific growth rate (μₘₐₓ) at which point cell washout occurs. In order to harvest the fermentation broth continuously and maintain constant culture volume, one end of the metal dip tube available in the bioreactor was set to a pre-determined volume mark while the other end was attached to a silicone tube, that was inserted into a peristaltic pump for withdrawing fermentation broth over the pre-set volume continuously. The feed rate (F, mL/hr) during continuous fermentation was calculated based on the pre-determined dilution rate (D=0.02 h⁻¹) and culture volume (V=2.5 L). The equation used to calculate feed flow rate in continuous phase is: F (mL/h)=V. D. 1000.

To achieve steady state and maintain a constant cell density during continuous fermentation, the feed rate was changed every 12 hours. This differs from Example 7 where the feed rate was constant during the continuous phase. A feed rate calculator was developed based on the targeted cell density (i.e., WCW at the end of fed-batch phase, constant), current feed rate (i.e., the rate at which the hybrid medium was fed for last 12 hours), current cell density (i.e., present WCW that is measured). The equation [D=(B*C)/A] in Table 22 was used to calculate the new feed rate every 12 hours. 10/18 is a conversion factor for instrument (i.e., 10% at 18 ml/hr). Each new feed rate meant that the dilution rate also was changed at the same time to account for the new rate. The range of harvested fermentation broth was 0.2 L-1.86 L in a 12 hour time span.

TABLE 22
Feed rate calculator used during continuous fermentation

| | Parameters | Values | Units | Notes |
|---|---|---|---|---|
| Inputs | Targeted cell | A | g/LWCW | This is the cell density at the end of fed- |
| | density | | | batch or beginning of continuous phase |
| | Current feed | B | mL/hr | The feed rate (%) used during last 12 |
| | rate | | | hours period |
| | Current cell | C | g/L | Cell density measured at present time |
| | density | | WCW | |
| Output | New feed rate | D = (B*C)/A | mL/hr | Revised feed rate for next 12 hours |
| | | | | period |
| | New feed rate | D*(10/18) | % | The feed rate (%) that needs to be set on |
| | | | | bioreactor's control panel |

Aside from maintaining constant cell density during continuous fermentation phase, another major goal was to increase the productivity of Euglena biomass using hybrid media. Hence, an investigation into the effects (i.e., to what extent of cells washout is observed, influence on metabolites profile) of growing Euglena at higher specific growth/dilution rate than the maximum specific growth/critical dilution rate was commenced. As per the Monod equation, it is known that the specific growth rate of an organism usually increases with nutrient concentration in the bioreactor. Hence, glucose concentrations were adjusted in the bioreactor ˜10 g/L by adding concentrated glucose solution (200 g/L) every 12 hours. The equation [D=(C−B)*(A/200)*1000] in Table 23 was used to determine the amount of concentrated glucose solution required to feed into the bioreactor.

TABLE 23
Concentrated glucose addition calculator used during continuous
fermentation

| Parameters | Values | Units | Notes |
|---|---|---|---|
| Current culture | A | L | 2.5 L, constant |
| volume | | | |
| Current glucose | B | g/L | Measured |
| concentration | | | |
| Target glucose | C | g/L | 10 g/L, constant |
| concentration | | | |
| Required volume | D = (C-B)*(A/200)*1000 | mL | Calculated |

3. Results & Discussion:

Although 200 mL seed inoculum was prepared, only 100 mL was added to 1.5 L of batch medium. The culture volume at the start of batch phase was 1.6 L. Prior to inoculating the seed culture, 600 μL of 2500× vitamin mix (i.e., the amount of vitamin required for 1.5 L batch medium) was added into the inoculation flask. The cell density at “0” hour of fermentation (i.e., just after seed inoculation) was OD₆₀₀˜2 and WCW˜3.7 g/L (Table 24). The glucose concentration at “0” hour was determined 13.5 g/L by YSI although it was supposed to be 15 g/L., which is due to the glucose concentration in the medium being diluted because of the addition of 100 mL seed inoculum into the bioreactor. After 36 hours of batch cultivation, the glucose concentration in the bioreactor was measured 5 g/L, and cell density was increased to OD₆₀₀˜12.57 and WCW˜22.75 g/L (Table 24). A quicker rate of glucose consumption was observed in this experiment due to the fact of inoculating higher concentration of seed inoculum at the start of fermentation.

TABLE 24
Growth of E. gracilis and culture conditions during continuous
fermentation using hybrid medium

| | | | | | Gax | Stirrer | pO2/ |
|---|---|---|---|---|---|---|---|
| Modes of | EFT | WCW | | Glucose | mix | speed, | DO |
| cultivation | (hr) | (g/L) | OD₆₀₀ | (g/L) | (%) | rpm | (%) |
| Batch | 0 | 3.70 | 2.02 | 13.50 | 21 | 300 | 64.9 |
| | 12 | 7.60 | 3.42 | 11.90 | 21 | 300 | 29 |
| | 24 | 14.45 | 6.25 | 10.35 | 21 | 336 | 19.8 |
| | 36 | 22.75 | 12.57 | 5.00 | 21 | 396 | 19.8 |
| Fed-batch | 48 | 28.50 | 14.70 | 2.62 | 21 | 478 | 19.6 |
| | 60 | 36.30 | 26.40 | 1.00 | 21 | 519 | 22.4 |
| | 72 | 38.55 | 26.67 | 0.36 | 21 | 376 | 21 |
| | 84 | 34.95 | 32.49 | 0.06 | 21 | 300 | 35.4 |
| Continuous | 96 | 27.70 | 24.96 | 0.24 | 21 | 301 | 31.5 |
| using recycled | 108 | 30.05 | 25.38 | 0.16 | 21 | 300 | 25.5 |
| medium (no | 121 | 34.55 | 21.18 | 0.19 | 21 | 300 | 33.4 |
| Glucose top up) | 132 | 28.05 | 25.59 | 0.26 | 21 | 300 | 34.8 |
| (1:1) | 148 | 27.55 | 20.46 | 0.22 | 21 | 300 | 53.5 |
| | 156 | 33.15 | 20.48 | 0.20 | 21 | 600 | 93.8 |
| | 168 | 31.25 | 19.28 | 0.17 | 21 | 600 | 95.5 |
| | 180 | 37.55 | 20.74 | 0.21 | 21 | 600 | 96.3 |
| | 192 | 32.05 | 19.02 | 0.16 | 21 | 600 | 95.8 |
| | 204 | 34.05 | 16.24 | 0.21 | 21 | 600 | 96.5 |
| Continuous (1:1) | 216 | 41.3 | 18.82 | 1.37 | 21 | 600 | 73.9 |
| top up with | 228 | 46.95 | 29.43 | 0.94 | 21 | 600 | 70.6 |
| glucose | 240 | 50.4 | 25.05 | 0.82 | 21 | 600 | 67.8 |
| | 252 | 57.8 | 24.18 | 1.04 | 21 | 600 | 62.3 |
| | 264 | 54.1 | 21.7 | 4.34 | 21 | 600 | 33.6 |
| | 276 | 48.45 | 18.84 | 3.55 | 21 | 600 | 13.6 |
| | 288 | 37.8 | 16.85 | 8.75 | 21 | 600 | 20.6 |
| | 300 | 37.8 | 19.16 | 3.44 | 21 | 600 | 32.1 |
| | 312 | 35.75 | 18.74 | 5.59 | 21 | 600 | 20.6 |
| | 324 | 34.05 | 16.42 | 5.466 | 21 | 600 | 19.5 |
| | 336 | 29.95 | 14.12 | 6.100 | 21 | 600 | 19.8 |
| | 348 | 29.5 | 17.7 | 4.826 | 21 | 601 | 20.2 |
| | 360 | 33.8 | 16.5 | 4.667 | 21 | 600 | 19.5 |
| | 372 | 32.95 | 16.98 | 4.145 | 21 | 600 | 22.9 |
| | 384 | 35.2 | 14.84 | 4.467 | 21 | 600 | 31.7 |
| | 396 | 29.15 | 13.06 | 5.583 | 21 | 600 | 36.5 |
| | 408 | 36.45 | 12.3 | 5.717 | 21 | 600 | 45.9 |
| | 420 | 32.65 | 9.98 | 6.141 | 21 | 600 | 49.5 |
| | 432 | 25.7 | 8.9 | 6.820 | 21 | 600 | 56.9 |
| | 444 | 24.8 | 9.44 | 6.540 | 21 | 600 | 62.8 |
| | 456 | 28.75 | 8.07 | 7.304 | 21 | 600 | 65 |
| | 468 | 28.2 | 8.77 | 6.272 | 21 | 600 | 66.8 |
| | 480 | 31.35 | 9.195 | 7.040 | 21 | 600 | 66.7 |

At the end of batch phase (at 36 hours), however, feed medium started to be added at a constant feed rate of 52.1 mL/hr so that cell density in the bioreactor could be increased further. A total of 2.5 L feed medium was added over 48 hours period to reach a culture volume to 4 L. In this experiment, specific glucose/substrate uptake rate of 0.07 gglu/gDCW/hr was set for calculating the feed rate although a value of 0.05 gglu/gDCW/hr was considered for all previous fed-batch experiments conducted in the laboratory. The reason for considering a higher value for specific glucose uptake rate was to provide cells sufficient amount of glucose to be grown at their maximum growth rate. Hence, a cell density of WCW˜35 g/L was achieved at the end of fed-batch phase (i.e., at 84 hours of cultivation) (Table 24). Nevertheless, a higher cell density of WCW˜38.6 at 72 hours was observed (Table 24), which confirmed that cell density was reduced due to unavailability of glucose in the bioreactor. Furthermore, this data shows that the residual glucose concentration during fed-batch phase was low (Table 24) even after considering a higher value of specific glucose uptake rate. This result suggests using feed medium with a higher concentration of glucose.

At the end of fed-batch phase (at 84 hours), ˜1.5 L fermentation broth was aseptically harvested using a peristaltic pump while confirming the culture volume in the bioreactor was 2.5 L. In order to ensure sterility, one end of a silicone tube was connected to the dip tube available in the bioreactor and the other end was connected to a harvest vessel. The harvested fermentation broth was then aseptically transferred to a sterile shake flask, centrifuged to recover recycled medium, and mixed with the fresh feed medium in the ratio of 1:1. The hybrid medium was then added into the bioreactor at a rate of 50 mL/hour to meet the pre-set dilution rate of 0.02 h⁻¹ and harvested fermentation broth at the same rate to maintain constant culture volume in the bioreactor. In this study, the feed rate was changed every 12 hours with a goal to maintain a constant cell density (i.e., WCW at the end of fed-batch phase) during continuous fermentation. However, it was observed that the feed rates decreased (from 50 mL/hour to 17.2 mL/hour) continuously during 84˜204 hours of cultivation (Table 25). Due to this, it was found that the dilution rates at those corresponding points also decreased. Nevertheless, apart from the few initial sampling points, cell density was able to be maintained close to the initial value (35 g/L WCW at 84 hours) (Table 24).

It was observed that the residual glucose concentration during 84˜204 hours of cultivation was almost zero (Table 24). The question of whether cell growth was hampered due to the unavailability of a sufficient amount of glucose in the bioreactor was then considered. As ensuring high productivity was of prime interest, it was decided to add concentrated glucose (200 g/L) to maintain glucose level ˜10 g/L and ensure that cell growth was not limited due to lack of carbon source in the bioreactor. Concentrated glucose was added for the first time at 204 hours along with hybrid medium at a particular feed rate. As a result, an increase to the feed rate/dilution rate was observed as soon as glucose concentration in the bioreactor ˜10 g/L was maintained at each sampling time. Due to implementation of this approach, higher cell density and biomass productivity was eventually achieved. The highest cell density ˜58 g/L WCW (Table 24) was measured at 252 hours of fermentation and the highest productivity ˜2.3 gWCW/L/hour (˜0.74 gDCW/L/hour) (Table 25). Higher productivity (˜2 gWCW/L/hour) was maintained for 60 hours (i.e., from 276 to 336 hours) (Table 25). Furthermore, it was observed that cells washed out slowly, which eventually reduced cell density and biomass productivity.

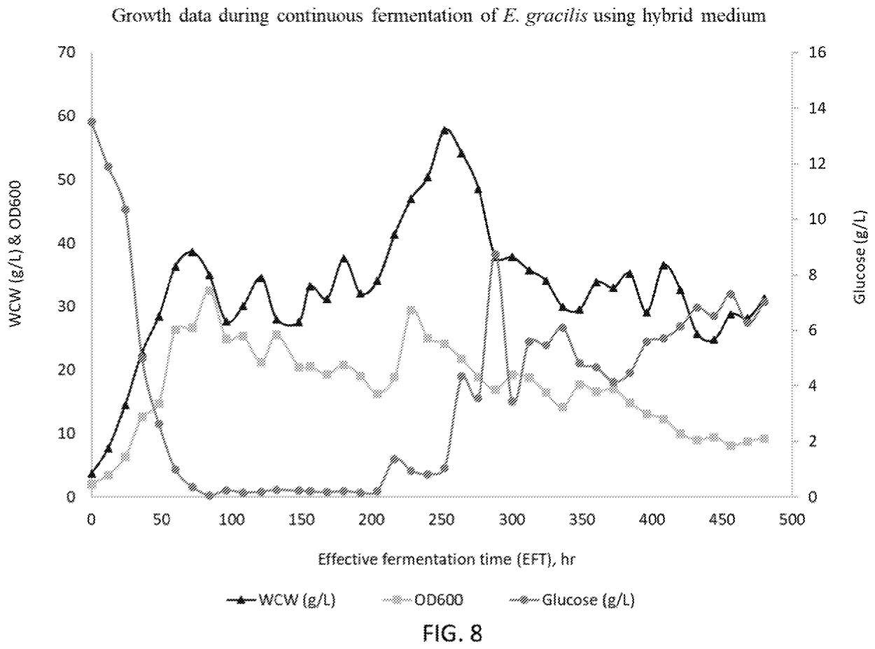

FIG. 8 shows the growth of Euglena gracilis during continuous fermentation on hybrid medium. Here, two major growth parameters (i.e., WCW, OD₆₀₀) of Euglena are presented along with a glucose consumption profile over the course of continuous cultivation. It was clearly observed that cell growth was limited from 72/84 to 204 hours due to unavailability of sufficient amount of glucose as hybrid medium was being supplied, which is estimated as containing ˜7.5 g/L glucose. In general, concentrated feed (5˜50 folds higher glucose concentration than batch medium) is used for fed-batch feeding. After realizing this potential fact, glucose in the bioreactor was maintained at ˜10 g/L by adding concentrated glucose solution (i.e., 200 g/L). The cell density was increased from 204 (34 g/L) to 252 (57.8 g/L) hours due to glucose feeding, however cell density started to drop again (Table 24). This might have occurred due to adding the hybrid medium as higher dilution rates, which resulted in cell wash out. In addition, these results show that the residual glucose level in the bioreactor started to increase from 252 hour.

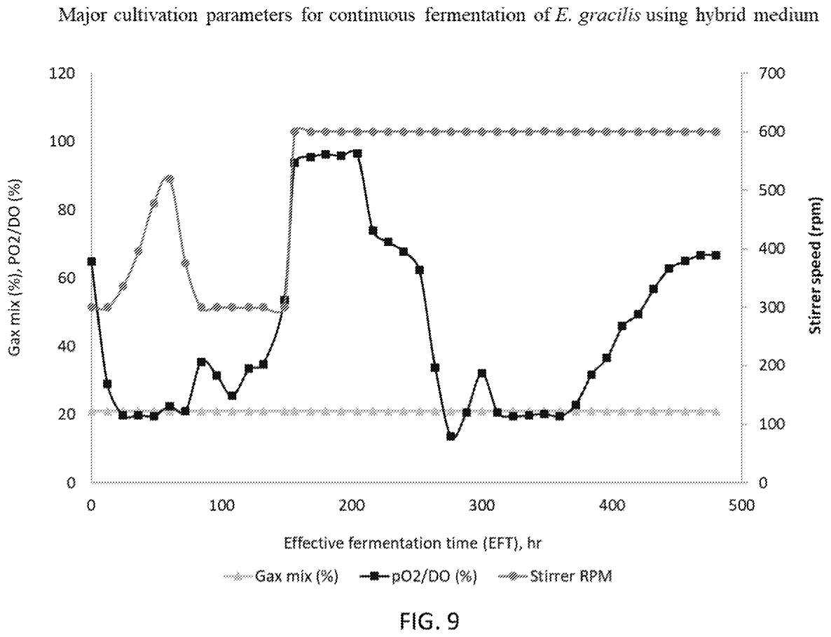

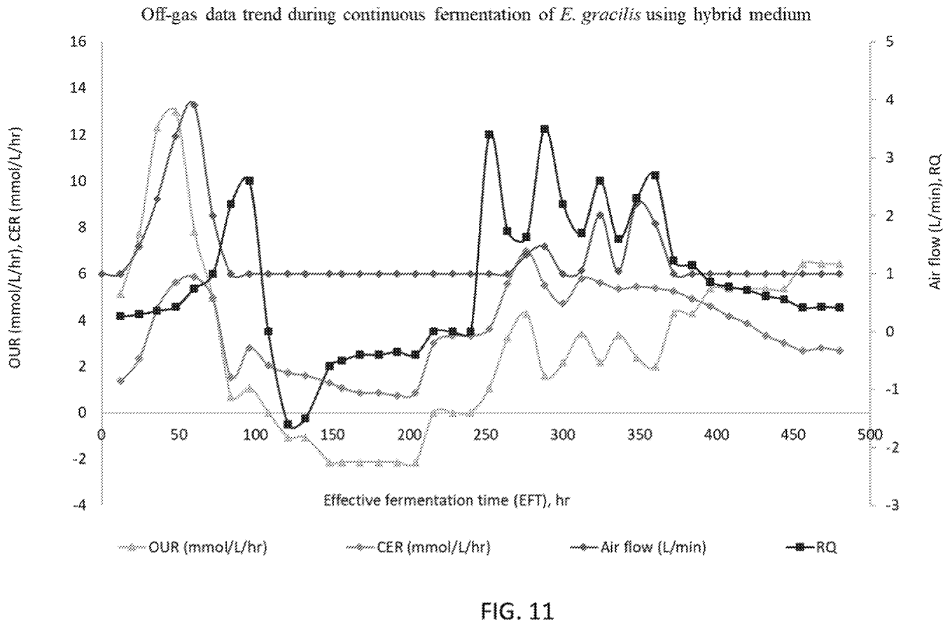

FIG. 9 shows the profile of major cultivation parameters involved during the continuous fermentation. In this study, as air was only supplied to control pO₂/DO level, the gas mix was automatically maintained at 21% (i.e., air contains approximately 21% O₂). In order to control pO₂/DO level to 20%, a cascade control was considered through the addition of air (1˜5 L/min) and agitation (300˜600 rpm). Although the cultivation was started at an agitation speed of 300 rpm, it increased almost to its maximum level during fed-batch and continuous feeding in order to meet the minimum pO₂/DO requirement (i.e., 20%). Similarly, air flow rate was automatically increased during the course of fermentation (FIG. 11). Any fermentation was typically stated at a pO₂/DO level close to 100% as the dissolved oxygen probe was calibrated to 100% using air before sterilizing the bioreactor. As per the pO₂/DO profile of this experiment, it was noticed that DO level at the start of inoculation was ˜65%, although this is not a significant concern. Nevertheless, it is required to mention that a sharp DO level decrease (i.e., 5˜10%) was often observed as soon as a seed inoculation into the bioreactor was carried out. According to this pO₂/DO profile, it is clearly observed that cells were deprived of glucose or any other carbon source from 108 to 204 hours. However, as soon as glucose concentration was maintained at ˜10 g/L by suppling concentrated glucose into the bioreactor, a slow decline in DO level towards the set point (i.e., 20%) was observed. Nevertheless, an increase in DO level was observed again from 372 hours even after maintaining the glucose concentration at ˜10 g/L. This likely occurred due to metabolic changes in cells (i.e., cells movement was very low, and growth was almost stopped).

TABLE 25
Key parameters for continuous fermentation of E. gracilis and
biomass productivity on hybrid medium

| | | | Harvested | Productivity | Productivity |
|---|---|---|---|---|---|
| | Feed rate | | biomass | (gWCW/ | (gDCW/ |
| EFT, hr | (mL/hr) | D (h-1) | (L) | L/hr) | L/hr) |
| 0 | | | | | |
| 12 | | | | | |
| 24 | | | | | |
| 36 | 52.1 | | | | |
| 48 | 52.1 | | | | |
| 60 | 52.1 | | | | |
| 72 | 52.1 | | | | |
| 84 | 50 | 0.020 | | | |
| 96 | 39.6 | 0.016 | 0.600 | 0.63 | 0.20 |
| 108 | 34 | 0.014 | 0.475 | 0.46 | 0.15 |
| 121 | 33.6 | 0.013 | 0.442 | 0.44 | 0.14 |
| 132 | 26.8 | 0.011 | 0.370 | 0.42 | 0.13 |
| 148 | 21.3 | 0.009 | 0.429 | 0.30 | 0.10 |
| 156 | 20.2 | 0.008 | 0.170 | 0.26 | 0.08 |
| 168 | 18 | 0.007 | 0.242 | 0.26 | 0.08 |
| 180 | 19.3 | 0.008 | 0.216 | 0.25 | 0.08 |
| 192 | 17.6 | 0.007 | 0.232 | 0.27 | 0.09 |
| 204 | 17.2 | 0.007 | 0.211 | 0.23 | 0.07 |
| 216 | 20.2 | 0.008 | 0.206 | 0.26 | 0.08 |
| 228 | 27 | 0.011 | 0.242 | 0.36 | 0.12 |
| 240 | 38.9 | 0.016 | 0.324 | 0.53 | 0.17 |
| 252 | 63.9 | 0.026 | 0.467 | 0.84 | 0.27 |
| 264 | 98.8 | 0.040 | 0.767 | 1.43 | 0.46 |
| 276 | 136.8 | 0.055 | 1.186 | 2.03 | 0.65 |
| 288 | 147.7 | 0.059 | 1.642 | 2.31 | 0.74 |
| 300 | 152.3 | 0.061 | 1.772 | 2.18 | 0.70 |
| 312 | 155.6 | 0.062 | 1.828 | 2.24 | 0.72 |
| 324 | 151.3 | 0.061 | 1.867 | 2.17 | 0.69 |
| 336 | 129.5 | 0.052 | 1.816 | 1.94 | 0.62 |
| 348 | 109.2 | 0.044 | 1.554 | 1.54 | 0.49 |
| 360 | 105.6 | 0.042 | 1.310 | 1.38 | 0.44 |
| 372 | 99.5 | 0.040 | 1.267 | 1.41 | 0.45 |
| 384 | 100 | 0.040 | 1.194 | 1.36 | 0.44 |
| 396 | 83.5 | 0.033 | 1.200 | 1.29 | 0.41 |
| 408 | 87.1 | 0.035 | 1.002 | 1.10 | 0.35 |
| 420 | 81.3 | 0.033 | 1.045 | 1.20 | 0.38 |
| 432 | 60 | 0.024 | 0.976 | 0.95 | 0.30 |
| 444 | 42.7 | 0.017 | 0.720 | 0.61 | 0.19 |
| 456 | 35.1 | 0.014 | 0.512 | 0.46 | 0.15 |
| 468 | 28.3 | 0.011 | 0.421 | 0.40 | 0.13 |
| 480 | N/A | N/A | 0.340 | 0.34 | 0.11 |

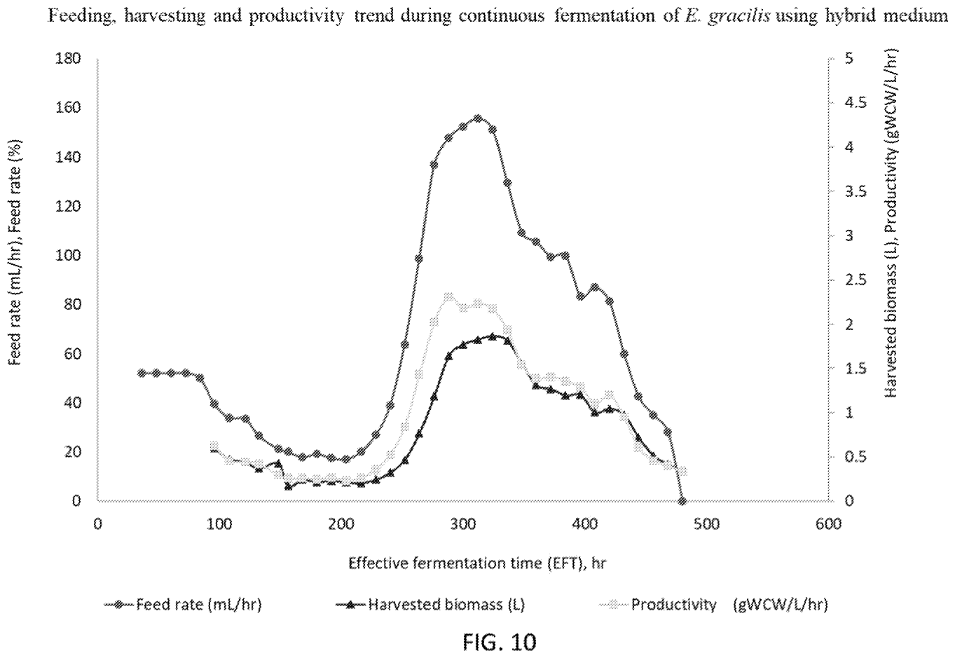

In this study, the feed rates were changed throughout the experiment in order to maintain a constant cell density in the bioreactor during continuous phase, which differs from Example 7 where they were constant. During fed-batch phase (from 36 to 84 hours), feed medium was added at a constant feed rate of 52.1 mL/hr (Table 25). However, feed rate of 50 mL/hr (i.e., dilution rate of 0.02 h⁻¹ as the culture volume was 2.5 L) was pre-set in order to start continuous feeding and harvesting. The feed rates were thereafter changed every 12 hours to maintain WCW of 35 g/L (i.e., cell density at the start of continuous phase). The results in FIG. 10 show feed rates were continuously decreased from 84 to 204 hours. However, feed rates started to increase when glucose concentration of ˜10 g/L was maintained in the bioreactor at each point of sampling. Nevertheless, after 324 hours of cultivation, it was not possible to keep feeding the culture at higher feed rates. It seems that the culture was overfed and cells were eventually washed out from the bioreactor. In this study, a total of 29.2 L of fermentation broth was harvested over the period of 20 days, although continuous feeding and harvesting was started at a surprising 2.5 L culture volume. This is one of the most important advantages of running continuous fermentation. In addition to this significant accomplishment, a significant biomass productivity was achieved. A productivity of 2.31 gWCW/L/hour (0.74 gDCW/L/hour) was achieved, which is the highest biomass productivity to date with hybrid media. The advantage of the continuous fermentation compared to a batch was amount of biomass harvested over the 20 days. The amount varied daily, however during times of high productivity, more biomass is harvested in order to keep the vessel at a steady state i.e. 35 WCWg/L. The higher productivity possibility due to the change of adapting our feed rate every 12 hours in accordance to the cell density, if the cell density was increasing, our dilution rate increased. Likewise, if the biomass was decreasing, the dilution rate or feed rate was decreased to try and keep it at that steady state. Previously, the dilution rate (feed rate) was fixed, meaning that we were not adapting for the difference in growth throughout time. By having the adaptive feeding, this kept up with the cell growth and increased productivity compared to previous runs like in example 7. In addition, concentrated glucose was added (after 204 hour time point), which had a direct impact on the cell biomass. These changes allowed for the harvest of more biomass and at a higher productivity than previously demonstrated.

FIG. 11 shows air flow and off gas profile during the continuous fermentation of E. gracilis using hybrid medium. In this study, air was supplied into the bioreactor through a cascade fashion, i.e., an air flow rate of 1˜5 L/min was set on the control panel and the system automatically adjusted its requirement in order to maintain minimum pO₂/DO level (i.e., 20%) in the bioreactor. This study shows a highest level of air (i.e., 3.9 L/min) was required during the fed-batch feeding phase where cells were growing in an exponential manner. In addition, it was observed that the stirrer speed was also high enough at 60 hours of cultivation although it reduced to minimum level (i.e., 300 rpm) until agitation speed was increased to 600 rpm at 156 hours (Table 24). However, these results show that the demand for air supply was minimal as soon as hybrid medium started to be added into the bioreactor. This may occur due to the unavailability of a sufficient amount of glucose in the medium. Nevertheless, air supply was automatically increased as soon as glucose level was maintained at ˜10 g/L in the bioreactor, although it was again dropped to a minimum level at 372 hours of cultivation. Based on the air flow and off-gas profile, it is clear that cells became inactive after 372 hours of cultivation. However, these result show the off-gas data (i.e., OUR, CER and RQ) depends on the air supplied into the system as OUR, CER and RQ were increased while higher amount of air was required and vice versa. OUR represents the oxygen uptake/utilization rate which is how many moles of O₂ consumed per litre of culture per hour. CER is carbon dioxide evolution rate which is how many moles of CO₂ produced per litre of culture per hour. RQ is respiratory quotient/coefficient where it is the ratio of the volume of carbon dioxide produced by Euglena to the volume of oxygen consumed by Euglena during respiration. Looking at the OUR profile, however, some negative values were obtained during continuous feeding of hybrid medium. This occurred due to measuring the exit O₂ values slightly higher than gas mix (i.e., 21%). The negative values were obtained at the start (121˜204 hr) of continuous feeding and harvesting, while glucose concentration in the bioreactor was very low due to feeding recycled medium (contained ˜7 g/L glucose). When it reached a maintaining glucose concentration ˜10 g/L, cells started using glucose, the Exit O₂ (%) values started decreasing (using oxygen), and Exit CO₂ (%) values were increased (producing CO₂) as a byproduct. Because Euglena is microalgae, it has the ability to use CO₂ as an energy source and produce O₂. In the case of off-gas analysis, it is a common trend where exit O₂ values are decreased, exit CO₂ will be increased and vice versa. More experiments are needed to determine the outcome where pure CO₂ is supplied into the bioreactor while organic carbon sources are reduced in bioreactor.

Without wishing to be bound by theory, one explanation for the observed increase in O₂ level and decrease in CO₂ amount could be that the heterotrophic Euglena are able to utilize the CO₂ as a carbon source under stressed or carbon-starved conditions, which is unexpected. This suggests that there is a pathway or that the energy generating pathway in Euglena is able to function in the plastid and not just in the fully functional chloroplast that is seen under light conditions. As a comparison, in the control experiment where there was not a carbon limitation, there were no observed negative values in O₂. Additionally it has been shown that heterotrophically grown E. gracilis cells are capable of fixing CO₂. This is typically done under limiting nutrient conditions and functions as a way to replenish TCA intermediates and can lead to generation of specific amino acids.

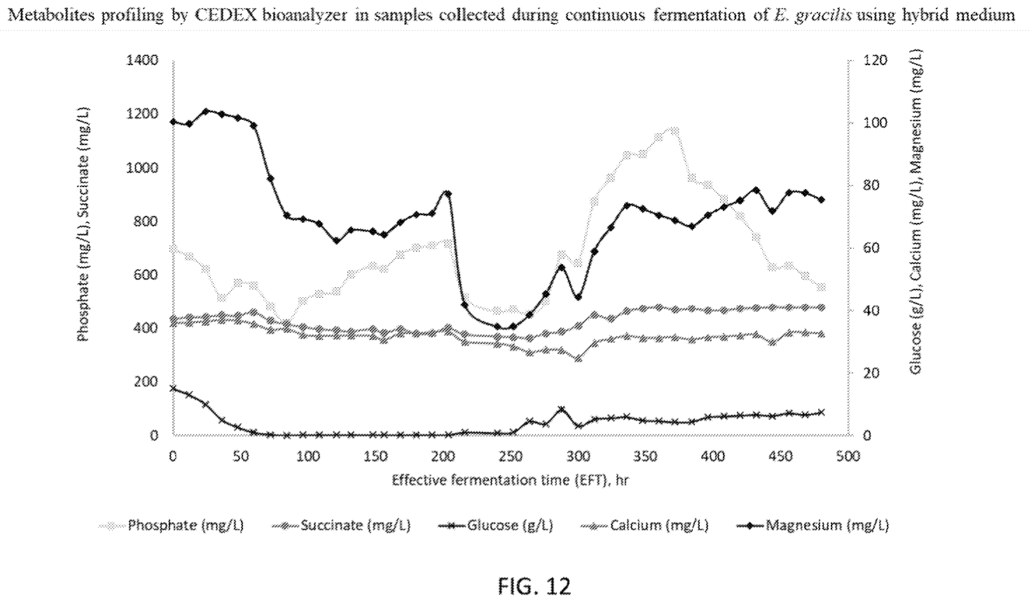

FIG. 12 shows the metabolite profile during continuous fermentation of E. gracilis. According to this data, not much consumption of Ca+ was observed during the course of fermentation, which pointed toward a possible reduction of CaSO₄ from the complex medium that was used in this experiment. The results of CaSO₄ optimization in chemically defined medium (CDM) conducted by our laboratory also confirmed this observation. Looking at this data, it is clearly observed that the cells consumed both phosphate and magnesium during batch and fed-batch phase. However, both components were seen to accumulate as soon as hybrid medium started to be added into the bioreactor. As such, both components were sharply reduced when glucose concentration was maintained at ˜10 g/L in the bioreactor. It seems that cells consumed both phosphate and magnesium rapidly in order to meet the growth rate due to the addition of glucose. However, both phosphate and magnesium again started to increase from 264 hours. Nevertheless, phosphate level again decreased from 372 hours, which is unexplained. Succinate/succinic acid content was also analyzed in all samples by HPLC. These results show that approximately 0.4 g/L of succinate was present from the start of the fermentation i.e. at 0 hour. While succinate was not added to our media, without wishing to be bound by theory, it may be for the yeast extract. 0.4 g/L has been seen in previous experiments, with slight variations over the course of the experiments. In this experiment, succinate's level was slightly reduced at some points of fermentation but increased back to about 0.4 g/L by the end of the fermentation. In addition, acetate, lactate, ethanol and pyruvate content were analyzed in all samples by CEDEX. However, the results showed that these were below the detection limits in all samples.

Continuous cultivation of Euglena gracilis as a control

1. Background

In this experiment, continuous fermentation with fresh media is used in a three-step process of batch, fed-batch, and continuous format. These results are compared to the recycled (hybrid) experiment above where hybrid media is added instead of fresh.

2. Methodology:

2.1. Maintenance of Mother Culture & Preparation of Seed Inoculum is the Same as Above for Hybrid Media in Example 8.

2.2. Continuous Fermentation:

A continuous/chemostat fermentation was conducted in this study in order to investigate whether cell growth/density can be maintained at a constant level while changing the dilution/feed rates every 12 hours of cultivation. An attempt was given to establish a steady state where cell growth occurs at a constant specific growth rate and all culture parameters (i.e., culture volume, dissolved oxygen concentration, nutrient and product concentration, pH, cell density etc.) remain constant. Before commencing this experiment, all media and associated stocks/reagents were prepared to accomplish the goal smoothly. A complex medium (i.e., contains glucose, yeast extract, ammonium sulfate, a range of salts, a range of vitamins, a range of trace metal salts, and vegetable oil, pH adjusted to 3.2) was used throughout the entire experiment (i.e., maintenance of mother culture, seed propagation and continuous fermentation). The composition of vitamin mix and trace metal mix is described in Tables 2 and 3, respectively and the composition of seed/batch/feed/complex medium is described in Table 1.

Continuous fermentation was initially started with a batch cultivation mode. The cell density of seed inoculum should be 20˜40 g/L WCW so that cell concentration at the onset (‘0’ hour) of fermentation is approximately OD₆₀₀ (optical density at 600 nm): 0.5˜2.0 or WCW: 2-4 g/L. The cultivation parameters of continuous fermentation are as follows: Temperature at 28° C., pH 3.2, agitation at 300-600 rpm, airflow rate of 0.4-2 vvm and 20% DO/pO₂. During fermentation, 30 mL samples were routinely collected every 12 hours. Right after sampling, samples were analyzed for cell morphology by microscopy, pH by pH meter, cell density by spectrophotometer (OD₆₀₀) and centrifugation of a known amount of culture broth (WCW), glucose concentration by YSI. Samples were further analyzed by CEDEX bioanalyzer and HPLC to determine metabolites concentration. Cell pellets obtained through WCW measurement were frozen at −80° C. until dry cell weight (DCW) of those samples was determined. Total solutes concentration in culture broth was also measured by freeze drying a known amount of supernatant (i.e., after removing cell pellets through centrifugation)

After running the fermentation for 36˜48 hours as batch mode while glucose concentration in the bioreactor was observed to be limiting (i.e., 0˜5 g/L), the cultivation was carried out for a further 2 days through fed-batch mode (i.e., feed medium was supplied into the bioreactor at a constant flow rate) before switching the cultivation to true continuous mode (i.e., continuous feeding and harvesting at a similar flow rate in order to maintain the culture volume constant). Once the glucose concentration in batch phase is close to 5 g/L, feed medium (i.e., contains 15 g/L glucose) was added at a constant flow rate without harvesting culture broth from the bioreactor. The flow rate of feed medium (F, mL/hour) was calculated using the exponential feeding formula, which is based on cell density (X=gDCW/L, measured initially as WCW that is multiplied by a factor of 0.32) and culture volume (V=L) at the end of batch phase and a constant specific glucose uptake rate (qₛ=0.07 gglu/gDCW/hr) and the concentration of glucose in feed medium (S_{f}=15 g/L). The equation used to calculate feed flow rate in fed-batch phase is as follows:

F (mL/hr)×1000

According to Monod equation, it has been well proven that the specific growth rate of an organism usually increases with nutrient concentration in the bioreactor. In addition, as it was observed many times a limited glucose level in the bioreactor during fed-batch fermentations of Euglena even after continuous addition of feeding medium through the above-discussed exponential feeding formula, it was decided to maintain glucose concentration in the bioreactor ˜10 g/L by adding concentrated glucose solution (200 g/L) every 12 hours of cultivation. This differs from the hybrid example where concentrated glucose was not fed until 204 hours of cultivation. The equation [h=(g−f)*(e/200)*1000] in Table 26 was used to determine the amount of concentrated glucose solution required to feed into the bioreactor during fed-batch and continuous feeding phases.

TABLE 26
Concentrated glucose addition calculator used during fed-batch &
continuous fermentation

| Parameters | Values | Units | Notes |
|---|---|---|---|
| Current culture volume | e | L | 2.5 L, constant |
| Current glucose concentration | f | g/L | Measured |
| Target glucose concentration | g | g/L | 10 g/L, constant |
| Required volume | h = (g-f)*(e/200) | mL | Calculated |
| | *1000 | | |

In this experiment, we set a dilution rate (D) of 0.03 h⁻¹ to start feeding, which is lower than the critical dilution (D_{crit})/maximum specific growth rate (μₘₐₓ) at which point cells washout is occurred. This differs from the hybrid media example as dilution rate was lowered to try to prevent washout. In order to harvest the fermentation broth continuously and maintain constant culture volume, one end of the metal dip tube available in bioreactor was set at the pre-determined volume mark and the other end was attached to a silicone tube, which was inserted into a peristaltic pump for withdrawing fermentation broth over the pre-set volume continuously. The feed rate (F, mL/hr) during continuous fermentation was calculated based on the pre-determined dilution rate (D=0.03 h⁻¹) and culture volume (V=2.5 L). The equation used to calculate feed flow rate in continuous phase is as follows:

F (mL/hr)=V.D.1000

In this study, the feed rate was changed every 12 hours in order to achieve steady state and maintain a constant cell density during continuous fermentation. Apart from maintaining constant cell density, one of our major goals was to increase the productivity of Euglena biomass. Hence, the effects (i.e., to what extent of cells washout is observed, influence on metabolites profile) of growing Euglena at higher specific growth/dilution rate than the maximum specific growth/critical dilution rate were investigated. A feed rate calculator was developed based on the targeted cell density (i.e., WCW at the end of fed-batch phase, constant), current feed rate (i.e., the rate at which the feed medium was fed for last 12 hours), current cell density (i.e., present WCW that is measured). The equation [d=(b*c)/a] in Table 27 was used to calculate new feed rate every 12 hours. Nevertheless, it is obvious that the dilution rates were changed simultaneously due to changing the feed rates.

TABLE 27
Feed rate calculator used during continuous fermentation

| | Parameters | Values | Units | Notes |
|---|---|---|---|---|
| Inputs | Targeted cell | a | g/LWCW | This is the cell density at the end of |
| | density | | | fed-batch or beginning of continuous |
| | | | | phase |
| | Current feed | b | mL/hr | The feed rate (%) used during last 12 |
| | rate | | | hours period |
| | Current cell | c | g/L | Cell density measured at present time |
| | density | | WCW | |
| Output | New feed rate | d = (b*c)/a | mL/hr | Revised feed rate for next 12 hours |
| | | | | period |
| | New feed rate | d*(10/18) | % | The feed rate (%) that needs to be set |
| | | | | on bioreactor's control panel |

3. Results & Discussion:

Although 200 mL of seed culture was prepared, only 100 mL to 1.5 L batch medium was inoculated. Hence, the culture volume at the start of batch phase was 1.6 L. Before inoculating seed culture, 600 μL 2500× vitamin mix (i.e., the amount of vitamin required for 1.5 L batch medium) was added into the inoculation flask. The cell density at “0” hour of fermentation (i.e., just after seed inoculation) was OD₆₀₀˜1.36 and WCW˜11.55 g/L (Table 28). There might be an error (i.e., probably water was not completely removed from the centrifuge tube) in measuring WCW of “0” hour sample as the WCW determined in a 12 hour sample was 3.6 g/L, which is fairly reasonable. The glucose concentration at “0” hour was determined to be 13.54 g/L by YSI although it was supposed to be 15 g/L. This is probably occurred due to the glucose concentration in the medium being diluted because of inoculating 100 mL seed inoculum into the bioreactor. After 36 hours of cultivation (i.e., at the end of batch phase), however, the glucose concentration in the bioreactor was measured 7.63 g/L and cell density was increased to OD₆₀₀˜8.89 and WCW˜16.2 g/L (Table 28).

TABLE 28
Growth of E. gracilis and culture conditions during continuous
fermentation using hybrid medium

| | | | | | Gas | Stirrer | pO2/ |
|---|---|---|---|---|---|---|---|
| Modes of | EFT | WCW | | Glucose | mix | speed, | DO |
| cultivation | (hr) | (g/L) | OD₆₀₀ | (g/L) | (%) | rpm | (%) |
| Batch | 0 | 11.55 | 1.362 | 13.540 | 21 | 300 | 98.7 |
| | 12 | 3.6 | 2.473 | 12.318 | 21 | 300 | 70.2 |
| | 24 | 12 | 4.3 | 11.500 | 21 | 300 | 35.5 |
| | 36 | 16.2 | 8.89 | 7.630 | 21 | 349 | 20.5 |
| Fed-batch | 48 | 23 | 12.86 | 5.055 | 21 | 416 | 19.5 |
| | 60 | 35.9 | 14.58 | 3.423 | 21 | 450 | 24.1 |
| | 72 | 52.15 | 32.25 | 3.408 | 21 | 504 | 18.6 |
| | 84 | 57.85 | 37.69 | 0.987 | 21 | 455 | 20 |
| Continuous | 96 | 65.35 | 35.63 | 1.632 | 21 | 600 | 19.4 |
| (1:1) top | 108 | 67.05 | 43.52 | 1.842 | 21 | 600 | 21.9 |
| up with | 120 | 58.8 | 33.52 | 4.794 | 21 | 600 | 18.5 |
| glucose | 132 | 67.45 | 44.65 | 2.279 | 21 | 600 | 45.3 |
| | 144 | 71.6 | 45.30 | 4.208 | 21 | 601 | 53.8 |
| | 156 | 67.85 | 44.94 | 10.333 | 21 | 601 | 20.4 |
| | 168 | 46.75 | 26.55 | 7.436 | 21 | 600 | 46.1 |
| | 180 | 49.35 | 24.24 | 4.470 | 21 | 600 | 46.7 |
| | 192 | 50.8 | 17.33 | 6.858 | 21 | 600 | 72 |
| | 204 | 32.65 | 17.52 | 7.465 | 21 | 600 | 55.2 |
| | 216 | 23.7 | 11.28 | 8.835 | 21 | 600 | 74.9 |
| | 228 | 22.8 | 7.90 | 9.000 | 21 | 600 | 83.4 |
| | 240 | 21.65 | 7.55 | 8.765 | 21 | 600 | 90.5 |
| | 252 | 20.15 | 8.13 | 10.146 | 21 | 600 | 83.6 |
| | 264 | 23 | 8.49 | 8.417 | 21 | 600 | 84.3 |
| | 276 | 25.4 | 8.66 | 9.563 | 21 | 600 | 82.1 |
| | 288 | 24.55 | 9.85 | 9.563 | 21 | 600 | 90.2 |
| | 300 | 23.65 | 11.78 | 7.564 | 21 | 600 | 90.1 |
| | 312 | 26.9 | 12.99 | 8.495 | 21 | 602 | 90.9 |

After completion of batch phase, feed medium was added at a constant feed rate of 52.1 mL/hour as the glucose concentration in the bioreactor was approaching ˜5 g/L. A total of 2.5 L feed medium was added over a 48 hour period to reach culture volume to 4 L. In this experiment, the specific glucose/substrate uptake rate of 0.07 gglu/gDCW/hr was set for calculating the feed rate although a value of 0.05 gglu/gDCW/hr was considered for all previous fed-batch experiments conducted in our laboratory. The reason for considering a higher value of specific glucose uptake rate was to provide cells sufficient amount of glucose to be grown at their maximum growth rate. In addition, a concentrated glucose solution (200 g/L) was added every 12 hours to maintain glucose concentration in the bioreactor at ˜10 g/L so that cell growth was not limited due to lack of glucose. Hence, a cell density of OD₆₀₀˜37.69 and WCW˜57.85 g/L was achieved at the end of fed-batch phase (i.e., at 84 hours of cultivation). Furthermore, it resulted in much higher cell density than that of other fed-batch fermentations conducted earlier. This result might have occurred due to adjusting glucose concentration in the bioreactor to ˜10 g/L every 12 hours of cultivation. Nevertheless, this data shows that the residual glucose concentration at the end of fed-batch phase was ˜1 g/L (Table 28) even after considering a higher specific glucose uptake rate and maintaining ˜10 g/L glucose in the bioreactor. This result suggests maintaining even higher than ˜10 g/L glucose during exponential feeding of fed-batch cultivations.

At the end of fed-batch phase, ˜1.5 L fermentation broth was harvested using a peristaltic pump while confirming the culture volume in the bioreactor was 2.5 L. In order to ensure sterility, one end of a silicone tubing was connected to the dip tube available in the bioreactor and the other end was connected to a harvest vessel. The feed medium was then added into the bioreactor at a rate of 75 mL/hour to meet the pre-set dilution rate of 0.03 h⁻¹ and harvested fermentation broth at the same rate to maintain culture volume constant. During continuous feeding phase, the feed rates were changed every 12 hours with a goal of maintaining a constant cell density of ˜50 g/L WCW, although the WCW at 84 hours of fermentation was 57.85 g/L. This is due to the WCW that was achieved during fed-batch phase. However, the feed rates were observed to increase (from 75 mL/hour to 180 mL/hour) continuously during 84˜168 hours of cultivation (Table 29). In fact, the feed rate was expected to be higher than 180 mL/hour from 120 hours of cultivation, however, the upper limit was 180 mL/hour given the restraints of the pump attached with the bioreactor.

This resulted in a cell density ˜71.6 g/L WCW (Table 29) and a productivity ˜5 gWCW/L/hour (˜1.6 gDCW/L/hour) (Table 29) at 144 hours of fermentation. In addition, a higher level of productivity (˜2 gWCW/L/hour) was maintained for 108 hours (i.e., from 96 to 204 hours of fermentation) (Table 29). It was initially assumed that these higher cell density and productivity were achieved due to adjusting glucose concentration ˜10 g/L in the bioreactor from the start of fed-batch phase. However, these productivity values were much higher than expected, and this promising result didn't match with the growth characteristics data (i.e., maximum specific growth rate, yield etc.) calculated earlier through the batch fermentation of Euglena. Nevertheless, it was noticed that the silicone tube (i.e., through which feed medium was added into the bioreactor) was attached to a wrong port of the 2^{nd} feeding bottle (i.e., the feeding bottle had to be changed during continuous feeding because only 7 L medium could be prepared in each 10 L bottle) that was connected with the bioreactor, which means feed medium was not pumped into the bioreactor for 25˜30 hours (i.e., approximately from 125 to 150 hours of fermentation), because this fault was identified after 25˜30 hours. However, the concentrated glucose solution (200 g/L) was added during that period, which resulted in high cell density and productivity. The higher productivity metric was not able to be maintained for a longer period of time due to wash out of the cells from the bioreactor. This wash out reduced cell density and biomass productivity.

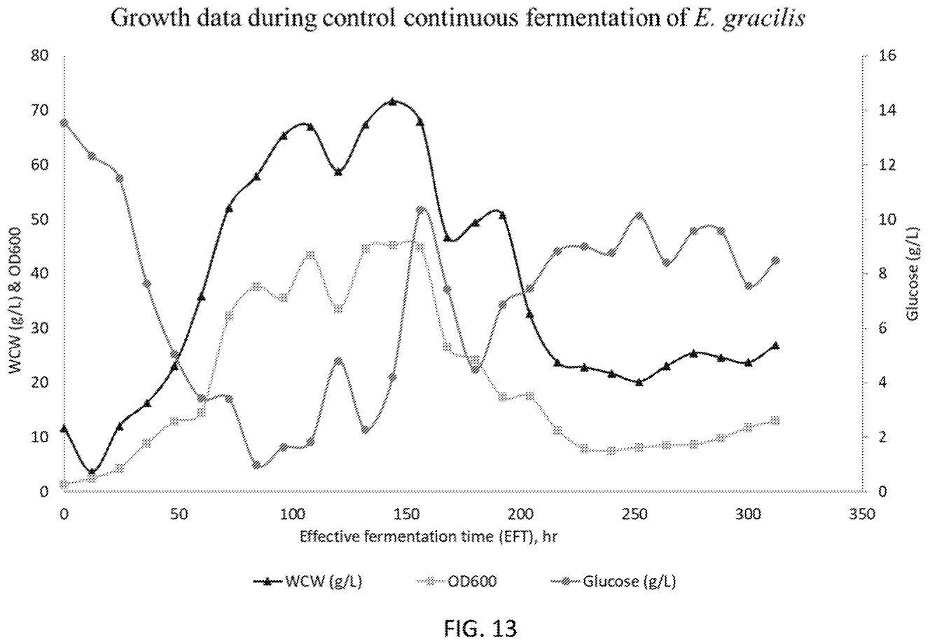

FIG. 13 shows 2 major growth parameters (i.e., WCW, OD₆₀₀) of Euglena along with glucose consumption profile over the course of continuous cultivation. We clearly observe that cell density (both OD₆₀₀ and WCW) was kept increasing till 144 hours of fermentation and started to decrease thereafter till to the end of fermentation. However, these results showed that cells were not stressed due to unavailability of glucose in the bioreactor as the residual glucose concentration was at least ˜2 g/L during continuous phase. However, cell density was sharply reduced from 156 hours of cultivation, which was likely due to supplying feed medium at high feed rates. The dilution rate that was set at 156 hours was 0.072 h⁻¹, which is much higher than the maximum specific growth rate (μₘₐₓ) of Euglena, and this probably resulted in cells wash out rapidly. Hence, a higher glucose level was determined in the bioreactor during the later phase of fermentation. However, there was no justification identified as to why cell density (i.e., both OD₆₀₀ and WCW values) at 120 hours suddenly dropped.

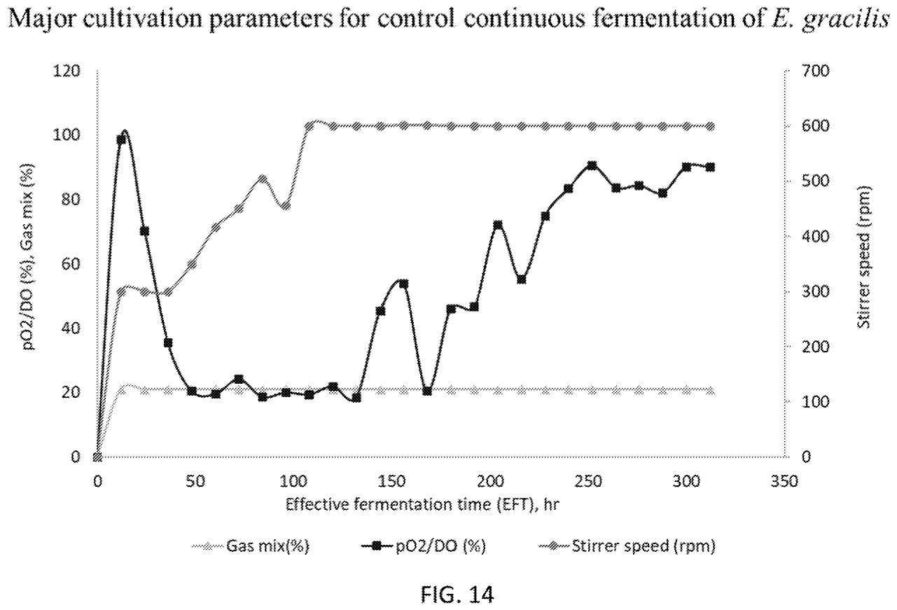

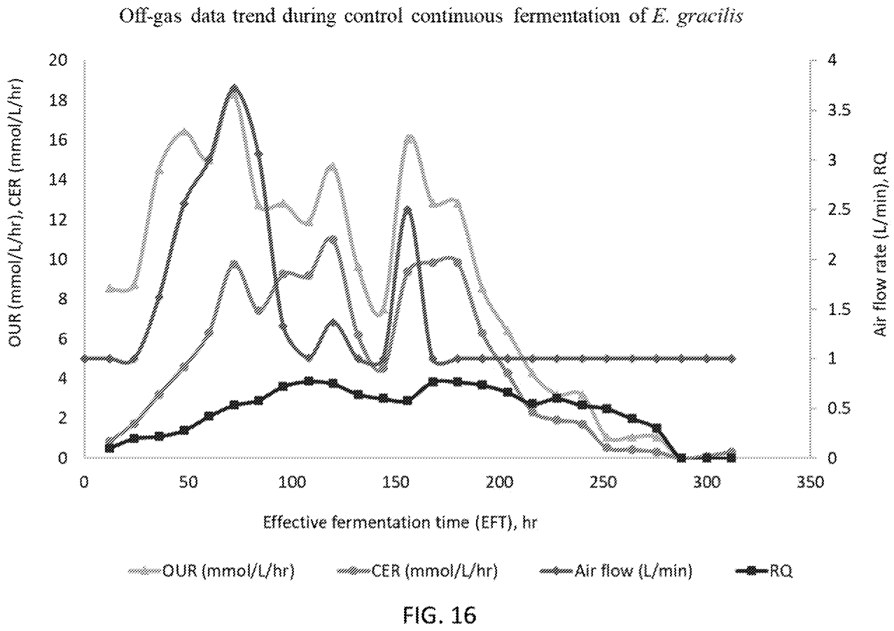

FIG. 14 shows the profile of major cultivation parameters involved during the continuous fermentation. In this study, the gas mix was maintained 21% (i.e., air contains approximately 21% O₂) as only atmospheric air was supplied to control pO₂/DO level. In order to control pO₂/DO level to 20%, a cascade control was considered through the addition of air (1˜5 L/min) and agitation (300˜600 rpm). Although the cultivation was started at an agitation speed of 300 rpm, it increased almost to its maximum level during fed-batch and continuous feeding in order to meet the minimum pO₂/DO requirement (i.e., 20%). Similarly, air flow rate was automatically increased or decreased during the course of fermentation (FIG. 16). FIG. 14 clearly shows that cells were growing in exponential pattern till 132 hours of fermentation. As soon as feed medium started to be supplied at high feed/dilution rates, cells wash out took place and the OUR of cells were reduced (FIG. 16), which resulted in increased pO₂/DO level in the bioreactor. There might be other metabolic changes that occurred in cells during this time.

TABLE 29
Key parameters for continuous fermentation of E. gracilis and
biomass productivity

| | Feed | | Harvested | Productivity | Productivity |
|---|---|---|---|---|---|
| EFT, | rate | | biomass | (gWCW/ | (gDCW/ |
| hr | (mL/hr) | D (h-1) | (L) | L/hr) | L/hr) |
| 0 | | | | | |
| 12 | | | | | |
| 24 | | | | | |
| 36 | 52.1 | | | | |
| 48 | 52.1 | | | | |
| 60 | 52.1 | | | | |
| 72 | 52.1 | | | | |
| 84 | 75 | 0.03 | | | |
| 96 | 98 | 0.0392 | 0.9 | 1.848 | 0.59136 |
| 108 | 132 | 0.0528 | 1.176 | 2.59504 | 0.8304128 |
| 121 | 157 | 0.0628 | 1.584 | 3.32244 | 1.0631808 |
| 132 | 180 | 0.072 | 1.884 | 3.96425 | 1.26856 |
| 144 | 180 | 0.072 | 2.16 | 5.0058 | 1.601856 |
| 156 | 180 | 0.072 | 2.16 | 5.0202 | 1.606464 |
| 168 | 168.3 | 0.06732 | 2.16 | 4.1256 | 1.320192 |
| 180 | 166 | 0.0664 | 2.0196 | 3.234726 | 1.03511232 |
| 192 | 168.5 | 0.0674 | 1.992 | 3.32498 | 1.0639936 |
| 204 | 110 | 0.044 | 2.022 | 2.812265 | 0.8999248 |
| 216 | 52 | 0.0208 | 1.32 | 1.2397 | 0.396704 |
| 228 | 23.8 | 0.00952 | 0.624 | 0.4836 | 0.154752 |
| 240 | 10.3 | 0.00412 | 0.2856 | 0.211582 | 0.06770624 |
| 252 | 4.67 | 0.001868 | 0.1236 | 0.086108 | 0.02755456 |
| 264 | 2.18 | 0.000872 | 0.05604 | 0.0403021 | 0.01289667 |
| 276 | 2.18 | 0.000872 | 0.02616 | 0.0211024 | 0.00675277 |
| 288 | 1.08 | 0.000432 | 0.02616 | 0.0217782 | 0.00696902 |
| 300 | 0.54 | 0.000216 | 0.01296 | 0.0104112 | 0.00333158 |
| 312 | 0.29 | 0.000116 | 0.00648 | 0.0054594 | 0.00174701 |

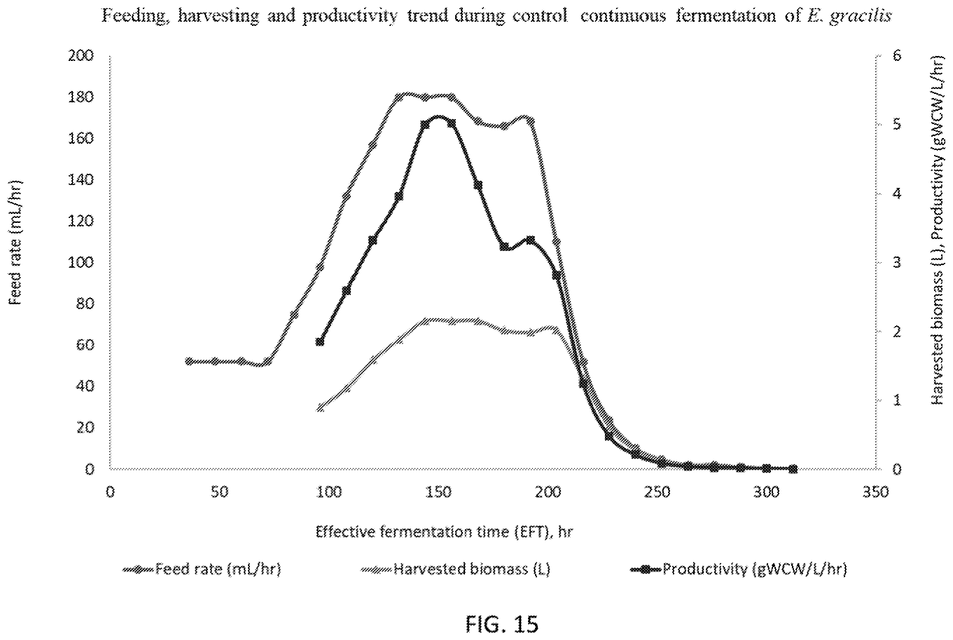

In this study, feed rates were changed throughout the experiment in order to maintain cell density in the bioreactor constant, which is an improvement from Example 4 where the feed rate was kept constant during continuous phase. During fed-batch phase (from 36 to 84 hours), feed medium was added at a constant feed rate of 52.1 mL/hr (Table 29). However, a feed rate of 75 mL/hr (i.e., dilution rate of 0.03 h⁻¹ as the culture volume was 2.5 L) was pre-set in order to start continuous feeding and harvesting. The feed rates were thereafter changed every 12 hours to maintain WCW of 50 g/L (i.e., although the cell density at the start of continuous phase was WCW˜57.85 g/L). The results in FIG. 15 show that feed rates were continuously increased from 84 to 132 hours. However, feed rates started to decrease sharply from 192 hours of fermentation. The productivity was observed to decrease from 156 hours cultivation. However, it was not possible to keep feeding the culture at higher feed rates after 192 hours of cultivation. It seems that the culture was overfed and cells were eventually washed out from the bioreactor. In this study, a total of 23 L fermentation broth was harvested over the period of 13 days, although continuous feeding and harvesting was started at 2.5 L culture volume. This is one of the most important advantages of running a continuous fermentation.

FIG. 16 shows air flow and off gas profile during the continuous fermentation of E. gracilis. In this study, air was supplied into the bioreactor through a cascade fashion i.e., an air flow rate of 1˜5 L/min was set on the control panel and the system automatically adjusted its requirement in order to maintain minimum pO₂/DO level (i.e., 20%) in the bioreactor. This study shows a highest level of air (i.e., 3.72 L/min) was required during the fed-batch feeding phase where cells were growing in an exponential manner. In addition, it was observed that the stirrer speed was also high enough at 72 hours of cultivation although it reduced a bit before agitation speed was increased to 600 rpm at 96 hours. These result shows that the demand of air supply was minimal as soon as we started diluting the culture rigorously. Based on the air flow and off-gas profile cells became inactive after 168 hours of cultivation. However, these result shows the off-gas data (i.e., OUR, CER and RQ) depends on the air supplied into the system as OUR, CER, and RQ were increased while a higher amount of air was required and vice versa.

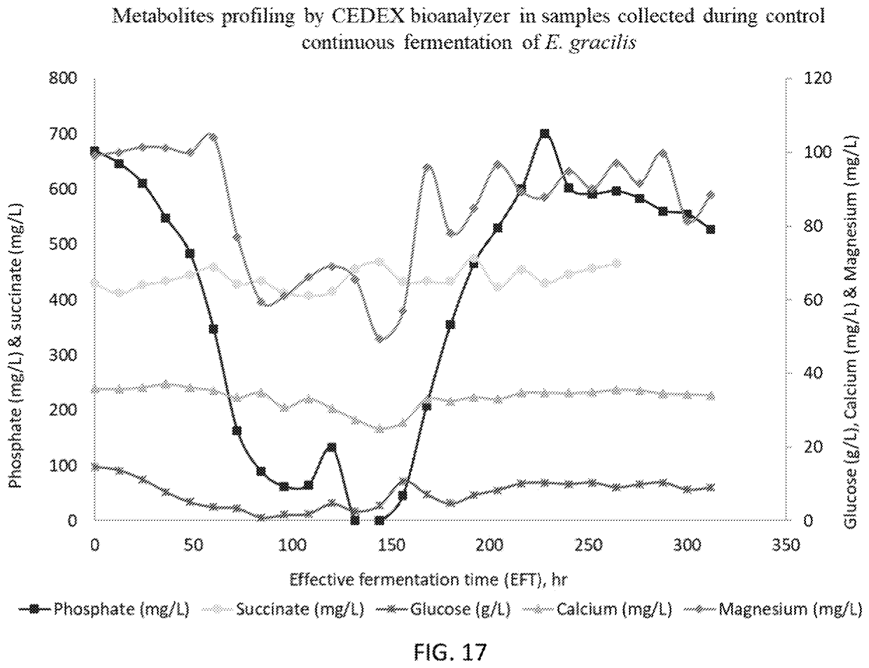

FIG. 17 shows the metabolites profiling during continuous fermentation of E. gracilis. According to this data, little consumption of Ca+ was observed (i.e., approximately 30% consumption after 144 hours as compared to the initial concentration at 0 hour of cultivation) during the course of fermentation, which indicates the possible reduction of CaSO₄ from the complex medium. Looking at this data, it was observed that cells consumed both phosphate and magnesium during batch and fed-batch phase. However, both components were seen to be accumulating from 144 hours of fermentation (i.e., during continuous feeding phase). This result probably occurred due to addition of feed medium at high feed rates. Beside this, there might be a metabolic shift, which resulted in slower rate of phosphate and magnesium consumption. Succinate/succinic acid content was also analyzed in all samples by HPLC. These results shows that approximately 0.4 g/L of succinate was present from the start of fermentation, which might be coming from yeast extract. In addition, acetate, lactate, ethanol, and pyruvate content in all samples was analyzed by CEDEX. However, the results showed that these were below the detection limits in all samples.

4. Conclusions:

This study confirmed that continuous cultivation of Euglena can be conducted at a range of dilution/feed rates using the standard complex medium that was used in the laboratory. Although an objective was to establish a steady state condition by changing feed rates every 12 hours, this data suggests this needs to be optimized if using a 12 hour sampling time point. If additional measurements are made every few hours, for example, or an online monitoring system is used, a steady state may be possible.

By running the continuous fermentation, it was possible to increase the overall productivity to 5 gWCW/L/hour (1.6 gDCW/L/hour), although there was an error while adding the feed medium into the bioreactor. However, the productivity metrics achieved through the study were much higher than expected as it did not match with the growth characteristics data (i.e., maximum specific growth rate, yield etc.) that was calculated earlier through the batch fermentation of Euglena. Nevertheless, it was possible to maintain a higher level of productivity (˜2 gWCW/L/hour) for 108 hours (i.e., from 96 to 204 hours of fermentation). This is due to an adaptive feeding style instead of keeping it constant, which in turn lead to a higher cell productivity. This can be seen by the differences in Example 4 where the feed rate was constant and this example where it matched cell growth. However, here, this productivity was not able to be maintained for a longer period of time. This is due to adding feed medium at a higher dilution rate than the maximum growth rate (μₘₐₓ), which resulted in cells washing out rapidly and reduced cell density and productivity.

Overall biomass yield was calculated on each input basis (gDCW/g inputs), which is approximately 34%. This is very similar to what was previously achieved from batch fermentations. However, this result is considerably higher than that achieved in fed-batch experiments that were previously performed in the laboratory. The reason for having low biomass yield on each input basis in the fed-batch fermentations is using 5× feeding medium, where 5× concentration of salts was added along with 5× concentrated glucose.

Mass Balance:

Another aspect that was measured is the inputs and outputs of the fermentation run. This included the oxygen gas (O₂) in and out, Carbon dioxide gas (CO₂) in and out, weight of feed materials (i.e., feed media), weight of fresh water in, weight of the recycled media used, and mass of the biomass out. These were calculated as follows:

$${O}_{2} In = \frac{( Liters of Air In ( L ) \times Percentage of {O}_{2} in ( \% ) )}{Liters per mole of ideal gas ( \frac{L}{mole} )} \times molecular weight of {O}_{2} ( \frac{g}{mole} ) ,$$

where liters of air in is the amount of air pumped in, percentage of Oxygen gas in is the assumed percentage in the atmospheric air, liters per mole of ideal gas is 22.4 L/mole, and the molecular weight of O₂ is 32 g/mole.

O₂ Out is calculated as follows:

$${O}_{2} Out = \frac{( Liters of Air In ( L ) \times Percentage of {O}_{2} Out ( \% ) )}{Liters per mole of ideal gas ( \frac{L}{mole} )} \times molecular weight of {O}_{2} ( \frac{g}{mole} ) ,$$

where liters of air in is the amount of air pumped in, percentage of Oxygen gas out is measured by the Blue Sense off-gas analyzer, liters per mole of ideal gas is 22.4 L/mole, and the molecular weight of O₂ is 32 g/mole.

The CO₂ in is measured by the following formula:

$${CO}_{2} In = \frac{( Liters of Air In ( L ) \times Percentage of {CO}_{2} in ( \% ) )}{Liters per mole of ideal gas ( \frac{L}{mole} )} \times molecular weight of {CO}_{2} ( \frac{g}{mole} ) ,$$

where liters of air in is the amount of air pumped in, percentage of Carbon dioxide gas in is the assumed percentage in the atmospheric air, liters per mole of ideal gas is 22.4 L/mole, and the molecular weight of CO₂ is 44.01 g/mole.

CO₂ Out is calculated as follows:

$$\begin{matrix}{CO}_{2} Out = \frac{( Liters of Air In ( L ) \times Percentage of {CO}_{2} in ( \% ) )}{Liters per mole of ideal gas ( \frac{L}{mole} )} \times molecular weight of {CO}_{2} ( \frac{g}{mole} ) , & \end{matrix}$$

where liters of air in is the amount of air pumped in, percentage of Carbon Dioxide gas out is measured by the Blue Sense off-gas analyzer, liters per mole of ideal gas is 22.4 L/mole, and the molecular weight of CO₂ is 44.01 g/mole. The Euglena dry biomass weight is determined by the Dry Cell Weight (DCW) which is the wet cell weight in grams multiplied by a conversion factor (0.32) that is based on the ratio between the wet and dry cell weights.

The Feed based on dry weight is calculated as follows:

$$\begin{matrix}Feed ( Dry Weight ) & = & ( Total Media Dry Weight ( \frac{g}{L} ) \times Total Fresh Media ( L ) ) & \\ & + & ( Total Glucose Feed Volume ( L ) \times Glucose Feed Concentration ( \frac{g}{L} ) , & \\ & & & \end{matrix}$$

where the total media dry weight is the mass of the total media in grams per liter, the total fresh media is the volume of media added, the total glucose feed volume is how much added and the glucose feed concentration is concentration of the glucose added in grams per liter.

The net yield of biomass generated per amount of feed added is calculated as follows:

$$Net Yield = \frac{Amount of dried biomass ( kg )}{Amount of Feed ( dry weight ) ( kg )} \times 100 \% ,$$

where the amount of dried biomass is the mass of the biomass, amount of feed (dry weight) is the mass of the feed (inputs, media) that was used to generate the biomass and it is multiplied by 100 in order to give the value as a percentage.

Water usage is calculated as follows:

$$Fresh water Use = \frac{Volume of fresh water used ( L )}{Mass of biomass generated ( kg )}$$

In addition, the amount of CO₂ used per amount of biomass generated is also calculated as follows:

$${CO}_{2} generation = \frac{( {CO}_{2} Out - {CO}_{2} In ) ( kg )}{Biomass Generated ( kg )} ,$$

where the CO₂ generation is the difference in the CO₂ out and the CO₂ in, divided by the amount of biomass generated to give you the amount of CO₂ produced per kg of biomass.

In Tables 30 and 31 below, the total amount of inputs and outputs for the fermentation runs (hybrid and control) is tabulated. From these numbers, the net yield, fresh water use and amount of CO₂ generated per unit of biomass is as follows: Hybrid: Net yield=37%, fresh water use=52.2 (L/kg of biomass) and kg of CO₂ generated per kg of biomass is 0.466.

In order to give significance to these numbers, a control run where no recycled media was added was conducted and the numbers for its fermentation run are as follows: Control: Net yield=34%, fresh water use=68.2 (L/kg of biomass) and kg of CO₂ generated per kg of biomass is 0.410.

When compared to the hybrid media run, the control has lower efficiency, used more water per kg of biomass generated, and had slightly less carbon dioxide produced than the hybrid media case. This suggests that the hybrid media approach is more efficient in its input usage, uses less water but generates very similar CO₂ amounts as the control continuous experiment.

TABLE 30
Mass balance calculation inputs for hybrid media fermentation run
and control fermentation run.

| Inputs | Hybrid Media (g) | Control (g) |
|---|---|---|
| Feed (dry weight) | 892 | 1038 |
| Fresh Water | 17,380 | 24,320 |
| O₂ In | 11,241 | 7,165 |
| CO₂ In | 29.4 | 19 |
| Recycled Media | 13,520 | — |

TABLE 31
Mass Balance outputs for calculation for hybrid media fermentation
run and control fermentation run.

| Fermentation | Hybrid Media (g) | Control (g) |
|---|---|---|
| O₂ Exhaust | 11,137 | 6,955 |
| CO₂ Exhaust | 184 | 165 |
| Dry Weight Euglena | 333 | 357 |

Furthermore, it is concluded that recycled medium may be used for the continuous cultivation of Euglena. These conclusions are drawn based on the demonstrated ability to run this fermentation for 3 weeks without any interruption. However, cell growth depends on the availability of a carbon and nitrogen source. Here, a complex nitrogen source (i.e., yeast extract) was used in the medium. Based on the glucose profile, it was observed that residual glucose level was very low during both fed-batch and initial continuous feeding phases of recycled medium. As such, the decision was made to include glucose at 204 hours of cultivation, which caused a change in the cell growth pattern when concentrated glucose was added into the bioreactor to maintain ˜10 g/L glucose. Although there was a desire to establish steady state conditions by changing feed rates every 12 hours, this data shows that it will need to be optimized if based on a 12 hour sampling time point. If additional measurements are made every few hours, for example, or an online monitoring system is used, a steady state may be observed. Additional experiments using recycled medium containing sufficient amount of glucose are needed before drawing any conclusion in this regard.

By running the continuous fermentation, an increase in the overall productivity to 2.31 gWCW/L/hour (0.74 gDCW/L/hour) was achieved. Higher productivity (˜2 gWCW/L/hour) was maintained for only 60 hours (i.e., from 276 to 336 hours), which is thought to be the result of adding hybrid medium at a higher dilution rate than the maximum growth rate (μₘₐₓ) that resulted in cells washing out slowly and reduced cell density and productivity.

We calculated overall biomass yield on each input basis (gDCW/g inputs), which is approximately 37%. This is very similar to what was previously achieved from batch fermentations. However, this result is considerably higher than that was previously achieved in previous fed-batch experiments. A reason for having low biomass yield on each input basis in fed-batch fermentations is using a 5× feeding medium where 5× concentration of salts was added along with 5× concentrated glucose. Excess salts could be inhibiting the growth when only glucose and or a nitrogen source is needed at the time.

In terms of mass balance, the hybrid media run had better efficiency, used less water than the control, but had slightly higher CO₂ production. The hybrid example also produced oxygen, suggesting that it might have converted CO₂ into energy. Overall, the continuous hybrid media run showcased increased productivity, efficiency and used less water overall.

### Example 9: Effect of Organic Acids at Two Concentrations on Euglena Growth

In this Example, the effect of five different organic acids, at different concentrations (low and high) were tested in the presence and absence of glucose. The low level of organic acids was chosen to mimic conditions that were found when using recycled media in a hybrid media approach where low concentrations of organic acids were found in the media. The higher concentration was chosen to see the impact at a higher concentration and preference of Euglena to utilize it as a carbon source. According to metabolic theory, carbon sources can be grouped into two categories based on their entry point into metabolism and whether or not they are used successively or co-utilized: Group A (refer to Example 10) sources enter metabolism through a common entry point and are predominantly metabolized successively in order of cellular preference—a process that is commonly ascribed as catabolite repression (generally but not limited to sugars). Group B (refer to Example 10) sources enter metabolism at multiple points and can be co-utilized leading to increased growth rates and enhanced production of products (generally but not limited to organic acids).

Methodology:

Cell culture preparation was conducted as described previously. Briefly, 3 mL of actively growing E. gracilis seed inoculum was added into 125 mL flasks containing 50 mL of media as mentioned in Example 6 Media contained different combinations of carbon sources as shown in Table 32. The final cell count in the media was million cells/mL.

Fermentation was carried out at 28° C. for 120 hours with continuous shaking (120 rpm). Measurements were taken at 0, 24, 48 and 120 h. At 0 h, 48 h and 120 h, 12 mL of sample was taken. The sample was used to measure dry cell weight (DCW), optical density (OD600), cell count, % solid content, glucose, microscopic cellular morphology and organic acids concentrations. At 24 h, 8 mL of sample was taken out as solid content and DCW were not determined.

Analytical Methods:

Dry Cell Weight (DCW) was determined gravimetrically as described in Example 3. Glucose concentration was measured as determined in Example 3.

$$DCW = \frac{[ {( Weight of boat + dried biomass )}_{{Final}_{}} - Weight of {boat}_{Initial} ]}{5} \times 1000 g / L$$

Organic Acid: 1 mL of harvested biomass was centrifuged (14000 rpm; 1 min) and supernatant was obtained. CEDEX was used to determine acetic acid, pyruvic acid and lactic acid present in the supernatant. The concentrations of analyzed acids in the supernatant were determined by comparison with the standard run.

5 mL of supernatant was collected from each treatment and stored at −80° C. until analysis could take place. 2 mL of sample were filtered through a 0.2 μm filter and 5 cc syringe into running vials. Organic acid content was detected using HPLC. Agilent HPLC-1260 infinity system equipped with DAD and an Aminex HPLC Column of HPX-87H (300×7.8 mm) were used. The mobile phase was 5 mM sulfuric acid with a flow rate of 0.35 mL/min heated at 40° C. The DAD detector was set at 210 nm. 10 [symbol] L sample was directly injected after it was filtered through a 0.2 um syringe filter through an autosampler. Individual organic acid concentrations were calculated using calibration curves achieved from generated standard calibration curves using: Fumaric acid; Malate Standard for IC, Succinate Standard for IC, Pyruvic acid (Sigma Aldrich).

% Solid Content: Solid content was determined gravimetrically. 5 mL of biomass was centrifuged (5000 rpm; 10 mins) and the supernatant was transferred into a pre-weighed 15 mL falcon tube. The tube along with supernatant was reweighed and then freeze dried using LABCONCO vacuum freeze dryer at −87° C. At the end of the drying process, the tube and residual solid was weighed. Solid content was determined by using the following formula:

$$\% Solid Content ( g / g ) = \frac{[ {( Weight of tube + residue )}_{Freeze dried} - {( Weight of tube )}_{Initial} ]}{[ {( Weight of tube + biomass )}_{Initial}} \times 100$$

OD: 1 mL of biomass was added into the cuvette and OD was measured using a spectrophotometer at 600 nm.

TABLE 32
Composition of media
Media Composition

| Chemical name | Concentration (g/L) |
|---|---|
| Carbon source | See Table 33 |
| Yeast extract | 5 |
| Ammonium sulfate (AS) | 2 |
| KH₂PO₄ | 1 |
| MgSO₄•7H₂O | 1 |
| CaCl₂•2H₂O | 0.1 |
| Vitamin solution (2500X) | 0.4 mL/L |
| Mineral solution (500X) | 2 mL/L |

TABLE 33
Concentrations of glucose and/or acids added into the media. Each
treatment was conducted in duplicate.

| | Glucose | | |
|---|---|---|---|
| Carbon source | conc (g/L) | Other carbon conc. (g/L) | |
| Glucose (Control) | 15 | — | |
| Pyruvate + glucose | 15 | ~0.05 | (0.03-0.07) |
| Pyruvate + glucose | 15 | ~2 | (1.3-2) |
| Malic Acid + glucose | 15 | ~0.05 | (0.04-0.11) |
| Malic Acid + glucose | 15 | ~5 | (4.5-5) |
| Succinic Acid + glucose | 15 | ~0.05 | (0.01-0.06)* |
| Succinic Acid + glucose | 15 | ~5 | (4.5-5.2) |
| Lactic Acid + glucose | 15 | ~0.05 | (0.04-0.07) |
| Lactic Acid + glucose | 15 | ~5 | (3.6-5) |
| Fumaric Acid + glucose | 15 | ~0.0005 | (0.0005-0.0008) |
| Fumaric Acid + glucose | 15 | ~5 | (3.4-5) |
| Pyruvate only | — | ~0.05 | (0.03-0.07) |
| Pyruvate only | — | ~2 | (1.3-2) |
| Malic acid only | — | ~0.05 | (0.04-0.11) |
| Malic acid only | — | ~5 | (4.5-5) |
| Succinic acid only | — | ~0.05 | (0.01-0.06)* |
| Succinic acid only | — | ~5 | (4.5-5.2) |
| Lactic acid only | — | ~0.05 | (0.04-0.07) |
| Lactic acid only | — | ~5 | (3.6-5) |
| Fumaric acid only | — | ~0.0005 | (0.0005-0.0008) |
| Fumaric acid only | — | ~5 | (3.4-5) |

| | | |
|---|---|---|
| Negative control | — | — |

*Some organic acids, including succinic acid are inherently present in media and therefore may impact concentrations detected.

Conclusions

1^{st} Conclusion from Experiment: (i) Euglena gracilis Z can utilize different types of acids as a carbon source and (ii) Acid supplementation into the glucose containing media can improve the biomass production of Euglena gracilis Z and allow for co-utilization of carbon sources.

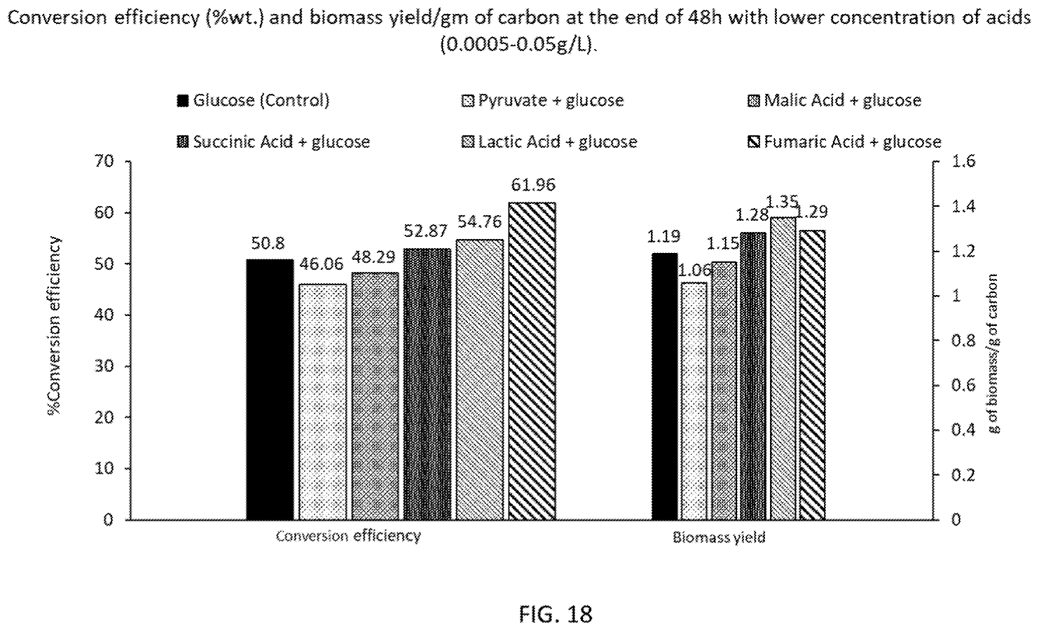

At lower concentrations (0.0005 g/L-0.05 g/L), none of the acids tested, either as a sole carbon or in combination with glucose (15 g/L), showed any inhibiting effect on the growth of Euglena gracilis Z (see Table 34). Compared to the negative control (no glucose), all of the acids alone gave a net biomass increase in the range of 20-100%. As compared to glucose alone (control), lactic and fumaric in combination with glucose gave 13.4% and 7.5% higher biomass respectively at the end of 48 h. At this concentration (i.e. 0.05 g/L), the conversion efficiency and biomass yield (g)/g of carbon with lactic acid & glucose containing media was 54.8% (˜8% higher than the glucose control) and 1.35 g/g of carbon (i.e. ˜13% higher than the control; FIG. 18). Similarly, fumaric acid (0.0005 g/L) in combination with glucose (15 g/L) had 21.9% higher conversion efficiency compared to glucose alone (15 g/L). Even though such a good conversion efficiency was seen with fumaric and glucose, biomass yield (g)/g of carbon was just 8.4% higher (see FIG. 18). This was due to the fact that the amount of fumaric acid (0.0005 g/L) added into the system was far less compared to glucose (15 g/L). So, even when there was complete consumption of fumaric acid and it contributed towards the improvement of biomass concentration, the contribution by the glucose portion in the media overshadowed its effect.

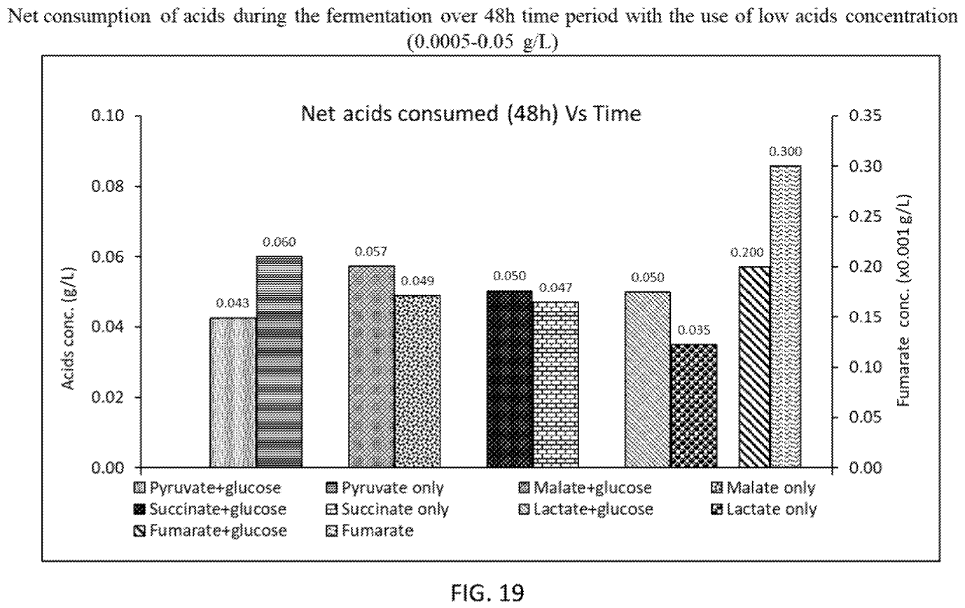

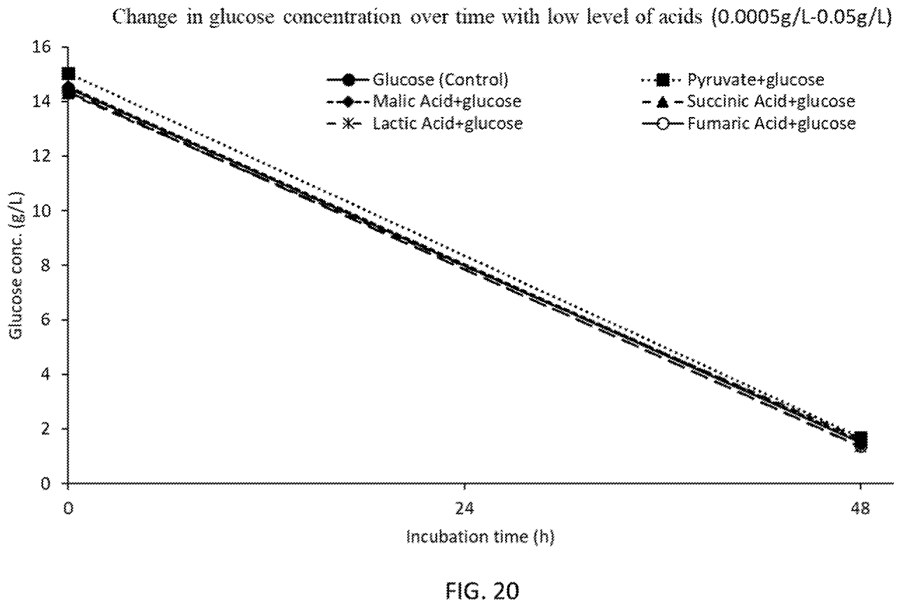

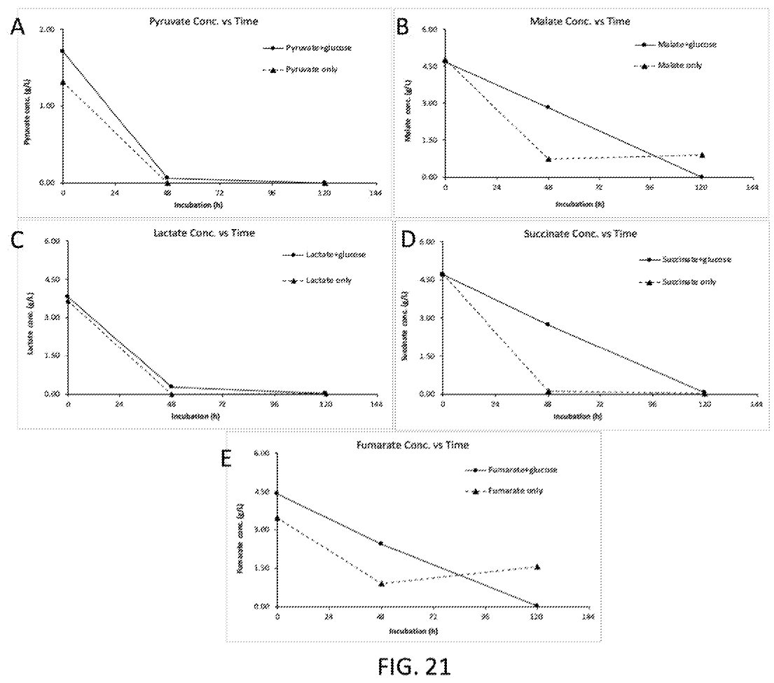

The consumption of all of the acids can also be seen from the graphs below (see FIG. 19; Table 35). Levels of organic acids were not detectable by the end of 48 h for lactate and pyruvate and these acid levels did not significantly affect the glucose uptake rate by the cells. Approximately 90% of glucose was consumed in all of the cases by the end of 48 h and a fairly similar level of glucose was consumed in all the cases (Refer FIG. 20) Similar results were obtained at higher concentrations of acid additions as seen in Table 36, FIGS. 21,22.

From FIGS. 19, 20, it can also be seen that, by the end of 48 h, all of the acids were consumed whereas some glucose was still left. From this, it can be concluded that Euglena gracilis Z simultaneously consumes small quantities of acids along with glucose rather than waiting for the major carbon source (glucose) to be exhausted.

In the presence of the organic acid alone, the organic acid is consumed as a sole carbon source (FIG. 19 and Tables 34 and 35). This aligns with reported literature which characterizes glucose and organic acids as separate carbon source groupings. When added in combination, glucose and the organic acid can be co-utilized (Tables 34 and 35; FIG. 21) and this type of fermentation has not been fully explored in E. gracilis until now. The presence of this flexible metabolism in E. gracilis allows for growth and product output to be directed on a wide variety of carbon/nitrogen sources.

TABLE 34
Net biomass change (g/L) at the end of 48 h with lower
concentration of acids.

| | | | Net |
|---|---|---|---|
| | Glucose | | biomass |
| | conc. | Acid conc. | conc. |
| Carbon source | (g/L) | (g/L) | (48 h) |
| Glucose (Control) | 15 | — | 6.70 |
| Pyruvate + glucose | 15 | 0.05 | 6.20 |
| Malic Acid + glucose | 15 | 0.05 | 6.50 |
| Succinic Acid + glucose | 15 | 0.05 | 7.00 |
| Lactic Acid + glucose | 15 | 0.05 | 7.60 |
| Fumaric Acid + glucose | 15 | 0.0005 | 7.20 |
| Negative control | — | — | 0.50 |
| (no carbon) | | | |
| Pyruvate only | — | 0.05 | 1.00 |
| Malic acid only | — | 0.05 | 0.60 |
| Succinic acid only | — | 0.05 | 0.70 |
| Lactic acid only | — | 0.05 | 0.60 |
| Fumaric acid only | — | 0.0005 | 0.70 |

TABLE 35
Change in organic acid concentrations (0.0005-0.05 g/L) during the
fermentation over 48 h time period (0.00 are the numbers that are
below the detection limit)
Change in acid conc. over time (g/L)

| | | Incubation (h) | |
|---|---|---|---|
| | Carbon source | 0 | 48 |
| Malate | Glucose (Control) | 0 | 0.0757 |
| | Malate + glucose | 0.103 | 0.1214 |
| | Malate only | 0.049 | 0 |
| | Control-gluc | 0 | 0 |
| Succinate | Glucose (Control) | 0.4884 | 0.6744 |
| | Succinate + glucose | 0.5135 | 0.6942 |
| | Succinate acid only | 0.5328 | 0.5042 |
| | Control-gluc | 0.4768 | 0.4953 |
| Fumarate | Glucose (Control) | 0.0003 | 0.0039 |
| | Fumarate + glucose | 0.0007 | 0.0041 |
| | Fumarate only | 0.0007 | 0.0003 |
| | Control-gluc | 0.0003 | 0.0002 |
| Pyruvate | Glucose (Control) | 0 | 0 |
| | Pyruvate + glucose | 0.04 | 0 |
| | Pyruvate only | 0.06 | 0 |
| | Control-gluc | 0 | 0 |
| Lactate | Glucose (Control) | 0.01 | 0 |
| | Lactic Acid + glucose | 0.06 | 0 |
| | Lactic acid only | 0.05 | 0 |
| | Control-gluc | 0.01 | 0 |

TABLE 36
Change in organic acid concentrations (2-5 g/L) during the
fermentation over 120 h time period
Change in acid conc. over time (g/L)

| | | Incubation (h) | | |
|---|---|---|---|---|
| | Carbon source | 0 | 48 | 120 |
| Malate | Glucose (Control) | 0.00 | 0.03 | 0.00 |
| | Malate + glucose | 4.67 | 2.84 | 0.00 |
| | Malate only | 4.78 | 0.73 | 0.90 |
| | Control-gluc | 0.00 | 0.00 | 0.00 |
| Succinate | Glucose (Control) | 0.43 | 0.65 | 0.72 |
| | Succinate + glucose | 5.15 | 3.37 | 0.77 |
| | Succinate acid only | 5.17 | 0.61 | 0.51 |
| | Control-gluc | 0.43 | 0.48 | 0.50 |
| Fumarate | Glucose (Control) | 0.00 | 0.00 | 0.00 |
| | Fumarate + glucose | 4.41 | 2.43 | 0.02 |
| | Fumarate only | 3.46 | 0.91 | 1.55 |
| | Control-gluc | 0.00 | 0.00 | 0.00 |
| Pyruvate | Glucose (Control) | 0.00 | 0.00 | 0.00 |
| | Pyruvate + glucose | 1.71 | 0.06 | 0.00 |
| | Pyruvate only | 1.31 | 0.00 | 0.00 |
| | Control-gluc | 0.00 | 0.00 | 0.00 |
| Lactate | Glucose (Control) | 0.01 | 0.00 | 0.00 |
| | Lactic Acid + glucose | 3.83 | 0.29 | 0.01 |
| | Lactic acid only | 3.67 | 0.00 | 0.02 |
| | Control-gluc | 0.01 | 0.00 | 0.00 |

When higher concentrations of acids were added into the media alone, most of the acids were consumed by the end of 48 h (FIG. 21; Table 36). At the end of 48 h, 0.90 g/L, 1.40 g/L, 2.20 g/L, 2.10 g/L and 2.10 g/L of net biomass media were respectively obtained using a media containing pyruvate (˜2 g/L), malate (˜5 g/L), succinate (˜5 g/L), lactate (˜5 g/L) or fumarate (˜5 g/L) as a sole carbon source. In the case of succinate and lactate, a further improvement in net biomass concentration by 13.6% and 9.5% respectively (compared to 48 h) were obtained by the end of 120 h (See Table 37).

TABLE 37
Net change in biomass over fermentation when higher levels
of acids (~2-5 g/L) were added solely or in combination with
glucose (~15 g/L)

| | | | % Increase |
|---|---|---|---|
| | | | in biomass |
| | Incubation time (h) | | between 48 h |
| Carbon source | 48 | 120 | and 120 h |
| Glucose (Control) | 6.50 | 6.50 | 0.0 |
| Pyruvate + glucose | 6.20 | 7.60 | 22.6 |
| Malic Acid + glucose | 6.80 | 8.70 | 27.9 |
| Succinic Acid + glucose | 6.70 | 8.30 | 23.9 |
| Latic Acid + glucose | 6.60 | 8.30 | 25.8 |
| Fumaric Acid + glucose | 7.30 | 9.50 | 30.1 |
| Negative Control (no carbon) | 0.60 | 0.60 | 0.0 |
| Pyruvate only | 0.90 | 0.80 | −11.1 |
| Malic acid only | 1.40 | 1.30 | −7.1 |
| Succinic acid only | 2.20 | 2.50 | 13.6 |
| Lactic acid only | 2.10 | 2.30 | 9.5 |
| Fumaric acid only | 2.10 | 2.20 | 4.8 |

When higher levels of acids (˜2-5 g/L) were added along with glucose (˜15 g/L), overall biomass concentration at the end of 48 h was higher compared to control (glucose alone). 4.6%, 3.1%, 1.5% and 12.3% higher biomass were respectively obtained when malic, succinic, lactic and fumaric were supplemented into the media containing 15 g/L of glucose (Table 37). The results were slightly different from what was obtained with lower acid supplementation. At lower acid supplementation (˜0.0005-0.05 g/L), malic acid, lactic, and fumaric acid produced −3% (low), 13.4% (high) and 7.5% (high) biomass concentration respectively; but at higher concentration (˜5 g/L), malic acid, lactic and fumaric respectively produced 4.6%, 1.5% and 12.3% more biomass compared to the glucose control. From this, it can be concluded that determining the optimum concentration of acid to be used along with glucose is important.

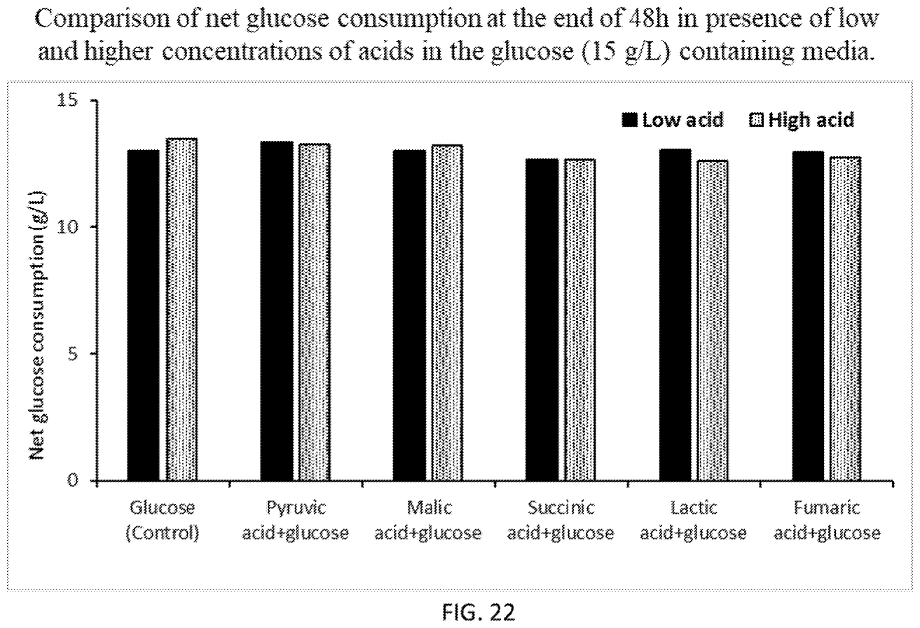

Glucose consumption in the presence of higher concentrations of acids during the first 48 h were quite similar to that at lower concentration of acids (see FIG. 22).

2^{nd} Conclusion from experiment: Euglena is capable of consuming glucose and acid together as a carbon source when supplied in combination with glucose:

At lower concentrations of acids: As already discussed above, the consumption of all of the acids can also be seen from Tables 36 and 36. All of the acids were depleted by the end of 48 h and these acid levels did not significantly affect the glucose uptake rate by the cells. Approximately 90% of glucose was consumed in all of the cases by the end of 48 h and the fairly similar level of glucose was consumed in all the cases (see FIG. 20, 23). From FIGS. 19 and 20, it can also be seen that, by the end of 48 h, all of the acids were consumed whereas some glucose was still left. From this, it can be concluded that Euglena gracilis Z simultaneously consumes small quantities of acids along with glucose rather than waiting for the major carbon source (glucose) to be exhausted.

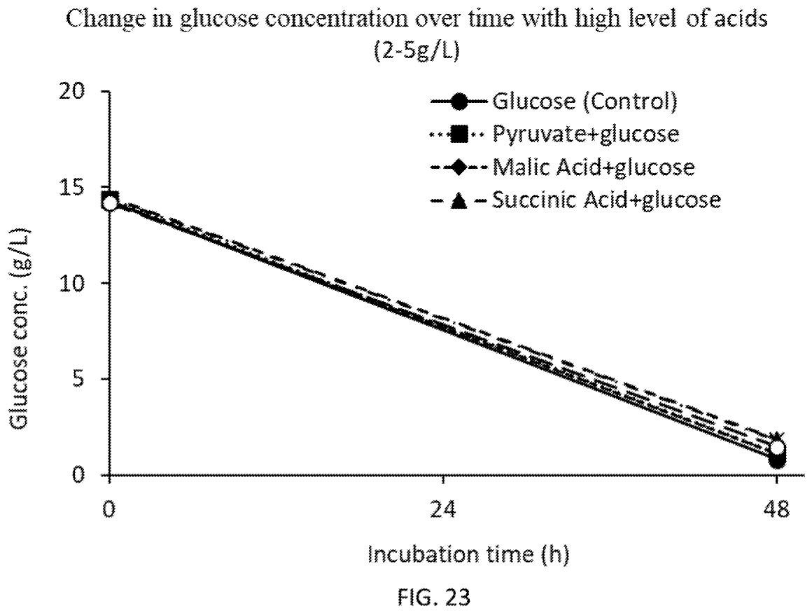

At higher concentrations of acid, at the end of 48 h, in all of the cases, some glucose is still left in the media whereas a certain level or all of the acid was consumed during this time. Like with lower concentrations of acids, even with higher concentrations, small quantities of acids are simultaneously consumed along with glucose (see FIG. 23).

3^{rd} conclusion from experiment: (i) At higher concentration of acids in glucose media, the growth takes place in two phases: glucose prominent phase (primary) and acid prominent phase (secondary). (ii) During the primary growth in glucose, Euglena gracilis Z may produce some growth promoting compounds or a metabolic pathway may be impacted, which improves the biomass production from acid during the secondary growth.

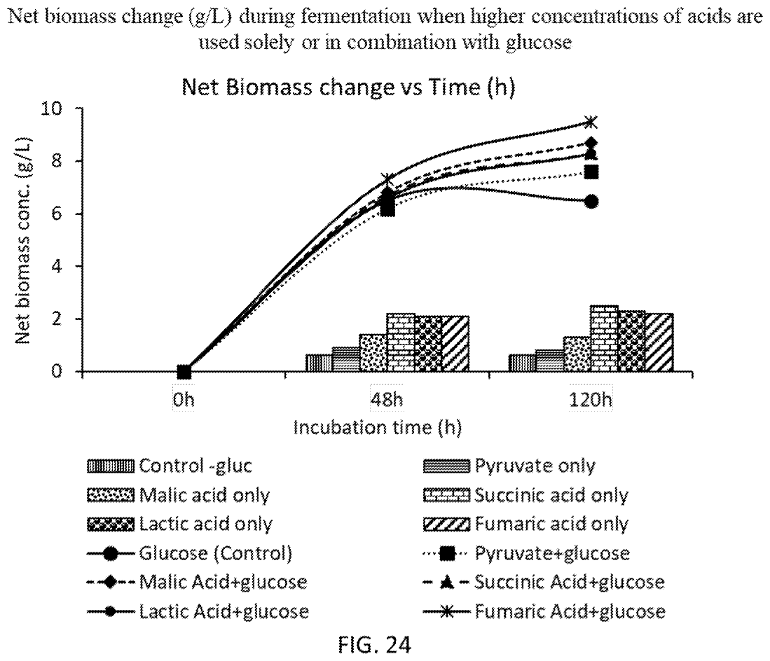

When higher concentrations of these acids are provided along with glucose, it consumes carbon (glucose & acids) in two phases. In the first phase, end of 48 h, it consumes glucose along with some amount of acid. During the second phase, between 48 h and 120 h, it utilizes acids as a carbon source for its survival and growth (see FIG. 24). Glucose and acid consumption profiles show that most of the glucose was consumed by the end of 48 h (FIG. 23). Also, during this phase some amount of acids were consumed. The amount of glucose and acids consumed were similar to what was obtained with lower concentrations of acid (Table 33 and 36, FIGS. 20, 23). Once glucose depleted, after 48 h, Euglena cells utilized acids as a carbon source (Table 36, FIG. 24). When higher concentrations of acids are solely fed, it starts to consume it as a carbon source right away.

To further understand the ability of Euglena to consume different concentrations of acids, we grew cells in different concentrations of fumaric acid (2 and 5 g/L). We found that by the end of 120 h, the microbe consumed almost all of the fumaric acid in both cases. Net fumaric acid consumption was determined at 48 h and 120 h for each treatment. The amount of fumaric acid consumed during the first 48 h when fed in combination with glucose was ˜48-67% whereas for fumaric acid alone it was ˜57-71%. By the end of 120 h, fumaric acid was completely utilized when fed along with glucose, at both the concentrations. However, only up to 77% of fumaric acid was consumed when fed alone. From this, it can be seen that the addition of glucose aids in the full utilization of fumaric acid overtime.

The maximum biomass obtained at 2 g/L and 5 g/L of fumaric acid was quite similar (i.e. ˜1.5 g/L). From this, we can tell that the level of fumaric acid to be used along with glucose should be optimized at lower concentrations (i.e. ≤2 g/L). Such optimization will possibly result in better synergistic effects of glucose and fumaric acid. This will subsequently give a higher or similar level of biomass compared to what we obtained when media with 15 g/L of glucose+5 g/L of fumaric acid was used. Obtaining a similar or higher level of biomass by using a lower level of acid is always preferable from an economic point of view.

Relative to the glucose control treatment with a conversion efficiency of 37.75% at 120 h; treatments with fumaric acid and glucose increased conversion efficiency of cells. However, regardless of fumaric acid concentration used (i.e. 2 g/L or 5 g/L), the conversion efficiencies remained similar. When 15 g/L glucose and 2 g/L of fumaric acid was used, the conversion efficiency was found to be 57.4%. Similarly, when 15 g/L glucose and 5 g/L of fumaric acid was used a conversion efficiency of 58.41% was obtained. This indicated that fumaric acid addition into the glucose containing media can improve the efficient conversion of inputs to biomass output. However, the level of acid to be added has to be further optimized.

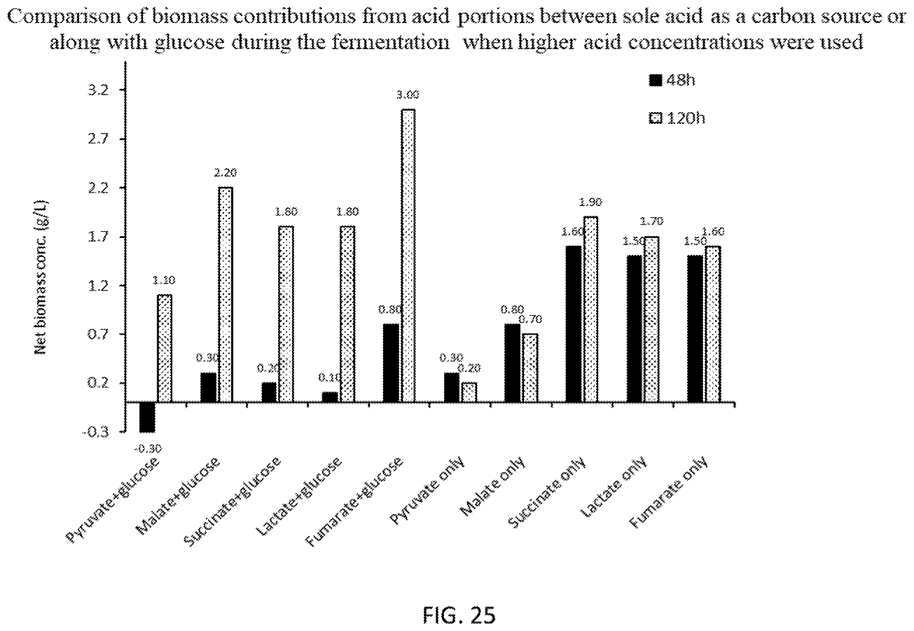

In a glucose media supplemented with higher concentrations of acids, at the end of 120 h, the total contribution from acids is higher compared to the acid alone contributions (FIG. 25). It means during the 1st phase, when glucose is consumed some other growth promoting compounds are also released which supports the growth from acids more effectively. Most of the acids (except succinic) were consumed and metabolized (acid to biomass production) better when fed in combination with glucose indicating that based on organic acid concentration the two carbon sources can be sequentially consumed one after another (diauxie) or simultaneously consumed (co-utilization). If we compare the contribution from acids at the end of 120 h with maximum acid alone contribution during the overall fermentation process (i.e. 48 or 120 h), it can be seen that pyruvate, malic, lactic and fumaric had 72.73%, 63.64%, 5.56% and 46.67% higher acid contribution when fed along with glucose.

### Example 10: Metabolic Theory for Utilization of Inputs by Euglena gracilis During Fermentation

Due to Euglena's remarkable metabolic capacity that allows it to grow in a wide range of conditions, it has been the subject of scientific inquiry for understanding fundamental aspects of biochemistry, physiology, evolution, anatomy and industrial potential.

Euglena can harness energy heterotrophically in aerobic and anaerobic conditions (intake of organic carbon sources for growth), mixotrophically (using a mix of different sources of energy for growth), and photo-autotrophically (obtaining carbon exclusively via CO₂ fixation) granting it unique status among microorganisms used in present day biotechnology.

Euglena's metabolic plasticity is a product of over a billion years of evolution whereby it has acquired and/or evolved biochemical pathways that permit survival under diverse environmental conditions. This is highlighted by the presence and in some cases redundancy of all central energy systems found throughout higher organisms including but not limited to glycolysis, gluconeogenesis, the tricarboxylic acid cycle (TCA), the pentose phosphate pathway (PPP) and the calvin cycle. Furthermore, Euglena has added pathways for fatty acids and wax esters, the anti-oxidant astaxanthin, vitamins and the major storage carbohydrate in Euglena, paramylon. Interestingly, Euglena appears to fix CO₂ in dark, heterotrophic conditions as a carbon source in carbon depleted and/or anoxic conditions.

A consequence of this diverse metabolic capacity is a seemingly limitless number of feedstocks for Euglena cultivation, and in this regard there is tremendous potential to utilize non-traditional feedstocks.

During heterotrophic fermentation (including but not limited to aerobic and/or anaerobic batch fermentation, aerobic and/or anaerobic fed-batch and/or repeated fed-batch, aerobic and/or anaerobic continuous fermentation, and/or aerobic and/or anaerobic recycled/batch or continuous fermentation), inputs are metabolized for the production of specific natural products. Natural products include but are not limited to: paramylon, protein, amino acids, wax esters, fatty acids, and vitamins.

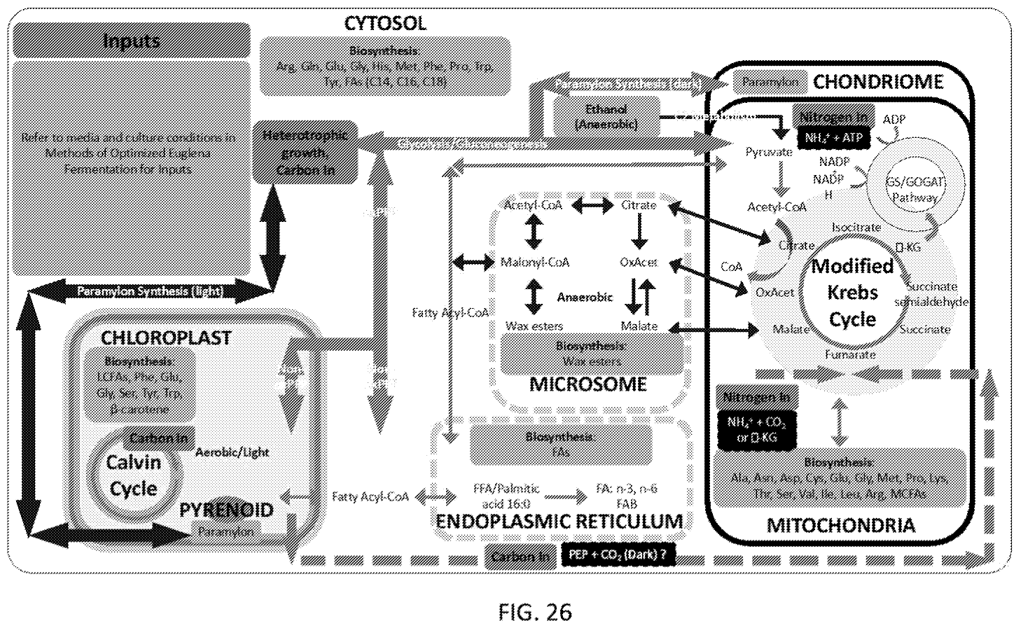

Under heterotrophic growth conditions, the carbon source is metabolized via glycolysis and/or gluconeogenesis and/or wax ester metabolism and/or fatty acid metabolism and/or amino acid metabolism and/or protein metabolism and/or paramylon metabolism. As an example, Pyruvate is oxidized and/or reduced in the mitochondria leading to the synthesis of amino acids and/or proteins and/or fatty acids and/or wax esters and/or glucose and/or paramylon and/or vitamins. Excess carbon is sequestered into the major carbon storage products of Euglena gracilis, namely paramylon and/or wax esters (FIG. 26). The quantity and ratio of end products (paramylon:fatty acids:proteins:amino acids:wax esters:vitamins) is governed by the carbon:nitrogen ratio (C:N ratio) utilized during growth and/or the growth parameters including but not limited to: pH, temperature, dissolved oxygen, dissolved CO₂, aeration, harvesting technique and fermentation technique (including but not limited to aerobic and/or anaerobic batch fermentation, aerobic and/or anaerobic fed-batch and/or repeated fed-batch, aerobic and/or anaerobic continuous fermentation, and/or aerobic and/or anaerobic recycled/batch or continuous fermentation). For example, high C:N ratios generally yield more storage products (paramylon and/or wax esters) and low C:N ratios generally yield more protein, amino acids and fatty acids. It is noteworthy that carbon sources can be grouped into two categories based on their entry point(s) into metabolism and/or whether or not they are used successively or co-utilized: Group A sources (including but not limited to mono, di and poly saccharides) enter metabolism through common entry point(s) and are predominantly metabolized successively in order of cellular preference—a process that is commonly ascribed as catabolite repression (generally but not limited to sugars and carbohydrates). Group B sources (including but not limited to organic acids) enter metabolism at multiple points and can be co-utilized with group A sources leading to increased growth rates and enhanced production of products (including but not limited to paramylon, fatty acids, proteins, amino acids, wax esters and vitamins).

### Example 11: Fed-Batch Fermentation of Euglena gracilis in 6 L Bioreactor

Objective: The main objective of this experiment was to optimize an exponential fed-batch feeding strategy for high cell density cultivation of E. gracilis. In addition, two most important growth parameters i.e., yield and productivity of Euglena at both batch and fed batch phases were determined. Beside this, the mass (input and output) balance for batch and fed batch cultivations of Euglena was also calculated.

Materials & Methods:

Preparation of seed inoculum: growth medium was used for seed propagation. A mother culture of E. gracilis, which has been cultivating for about 2-3 months, was fed once every 3-4 days with about 100-200 mL growth medium. 50 mL of this mother culture is used to inoculate a 500 L shake flask containing 150 mL growth medium. In addition, 0.08 mL of 2500× vitamin stock will be added to the culture. The resulting culture (total 200 mL) will be cultivated at 28° C. and 150 rpm for 3 days. On Day 3, inoculum status is checked by microscopy (actively moving, long elongated cells are best for inoculation) and the cell density will be determined by an automated cell counter. A seed inoculum with a cell density of approximately 25-30×10⁶ cells/mL is suitable for inoculation.

In this study, a growth base medium containing 15 g/L glucose and 5 g/L yeast extract was used as a batch medium. The batch cultivation was started with 2.5 L batch medium.

The above-mentioned materials were weighed for 2.5 L volume and dissolved accordingly in deionized water. The resulting medium was transferred into a 3 L bioreactor assembled with proper tubing. The bioreactor was then autoclaved at 121° C. for 30 minutes. After completion of autoclave when the medium was cooled down to room temperature, 1 mL the 2500× vitamin stock (new) was aseptically transferred into the bioreactor.

In this study, 5× concentrated batch medium was used as feed medium and 3 L of feed medium was prepared for fed batch fermentation. However, the required amount of yeast extract was separately dissolved up to 500 mL deionized water and transferred to a glass bottle. The rest of the materials were dissolved up to 2495 mL deionized water and transferred into the 3 L feeding bottle. All bottles containing feed medium were then autoclaved at 121° C. for 30 minutes. After completion of autoclave when the medium was cooled down to room temperature, 6 mL the 2500× vitamin stock (new) and 500 mL yeast extract solution were aseptically transferred into the feeding bottle.

Fed batch cultivation was started with batch fermentation. The seed inoculum (200 mL) cultivated for 3 days was transferred to an inoculation flask and aseptically inoculated into the bioreactor containing 2.5 L batch medium. It was observed that the cell density at the start of batch cultivation was approximately 1×10⁶ to about 3×10⁶ cells/mL.

The culture was continuously stirred at 70-100 rpm by a typical impeller and aerated with 1 L/min of air (0.4 vvm). The pH of the culture was maintained to 3.2 by supplying (automatic) 1 M NaOH. The dissolved oxygen was maintained 20% by supplying (automatic) pure oxygen into the bioreactor. Samples were aseptically collected from the bioreactor every day. Cell morphology was checked by microscope and cell growth was monitored by automated cell counter, spectrophotometer (optical density at 600 nm), wet cell weight (centrifugation) and dry cell weight (freeze dry). The glucose concentration was measured by YSI autoanalyzer.

However, the batch cultivation was run for 48 hours since it was observed that the glucose concentration in the bioreactor at this point dropped to below 5 g/L. Fed batch cultivation was then started with supplying feed medium (i.e., 5× concentrated of batch medium) into the bioreactor in order to maintain exponential growth. The feeding flow rate was calculated by considering the specific growth rate (μ=0.03 h⁻¹), yield of biomass (Yxs=0.7 g DCW/g), concentration of DCW in the bioreactor at 48 hours (X=9-10 g DCW/L) and the concentration of glucose in feed medium (75 g/L). The feeding flow rate (mL/hr) will be varied based on the concentration of cells in the bioreactor. The feed medium was added initially at a rate of 5.77 mL/L/hr. The feeding flow rate was daily increased to a final rate of 19.49 mL/L/hr after 120 hours of cultivation in proportion to the increase in biomass concentration in the bioreactor. Total of 3 L of feed medium was supplied in 3.5 days (from 48-130 hours).

Results and conclusion: The yield and the productivity of Euglena during batch phase were 0.35 g DCW/g input and 0.167 g/L/hr. In case of fed batch fermentation, the overall yield was dropped to 0.26 g DCW/g input but productivity was increased, i.e., 0.18 g/L/hr. Most interestingly, the productivity at only fed batch phase was increased to 0.575 g/L/hr, which is a common trend of fed batch fermentation.

In case of feeding process optimization, this data showed Euglena's growth rate can be maintained exponentially by using considering the specific growth rate of 0.03 h⁻¹ and biomass yield of 0.7 g DCW/g glucose.

### Example 12: Tank

An example embodiment of a bioreactor tank system is depicted in FIG. 27. The tank is merely an example consistent with disclosed embodiments and it should be understood that other tanks are within the scope of the disclosure. One embodiment is depicted in FIG. 28. FIG. 29 depicts a top view sparger grid that can be used in combination with FIG. 28.

An example tank includes a bubble column bioreactor for large-scale cultivation of Euglena is made of stainless steel and has a total maximum allowable volume of 17,000 L and a maximum allowable pressure is 0.33 bar (5 psig). The tank is not insulated but is equipped with three heating and cooling jacket shells. The construction and configuration of the bioreactor allows a safe sterilization cycle of the production vessel with saturated steam at 103° C.-107° C. at a pressure of approximately 4.3 psig or with peroxyacetic acid. The tank has an aspect ratio of 3. The tank has a total of 18 blind plug fittings. A two inch blind plug at the bottom of the vessel constitutes the vessel drain or the main harvest port through which the culture is transferred to the harvest transfer line and finally to the disk-stack centrifuge. At the top of the bubble column bioreactor, there are a total of 6 blind plug fittings. The tank has an independent main feed line connected to a two inch blind plug fitting at approximately two thirds of the vessel height. Concentrated media, cell inoculum, and fresh process water are fed to the bioreactor through this main feed line.

The tank includes an internal aerator/mixing system configured in a dual sparging mode consisting of one to three microspargers and two venturi nozzles through which clean compressed air is injected. Aeration is primarily performed by the microspargers, which provide oxygenation to the cultures inside of the tank. Oxygenation provided by the nozzles are considered to be minimal in comparison to that provided by the microspargers. The microspargers are designed to minimize cell shear (or damage) at high air flow rates by providing sufficient air sparging surface area depending on the average porosity of the sintered metal. In other words, the microspargers are design to generate gas entry velocities below a critical value for Euglena (e.g, below a value at which cell damage through shear that occurs at the surface of the microspargers). The lower pressure differential across the coarse spargers has led to reproducible and more productive growth because of the lower gas entry velocity. In turn, cell growth in larger production fermentation tanks (e.g., 20,000 L bioreactors) was previously hampered by the higher gas entry velocity through the smaller pores of the fine spargers.

In testing of the example bioreactor system, Euglena gracilis culture volumetric productivity in the 20,000 L bioreactor was increased two-fold by replacing 3 fine air spargers with a single coarse sparger. The higher-pressure differentials across the fine and the coarse sparger suggest the gas entry velocity may be too high through the fine sparger with the resulting local turbulence shearing and killing the cells.

The venturi nozzles provide the overall bulk mixing of the vessel and help to adjust or maintain the internal pressure of the tank. Although they are used primarily for oxygenation, the microspargers also contribute in part to the bulk mixing and the ascending fluid flow to efficiently resuspend cells. The internal venturi nozzles are tuned to create a heterogeneous aerobiosis regime in the bubble column bioreactor comprised of anaerobic and aerobic zones in Euglena cultures. The creation of the anaerobic and aerobic zones was confirmed by computational fluid dynamics studies based on the example bioreactor. For example, fluid dynamics studies showed that zones of high mixing are localized around the nozzles and the zones of low mixing are also formed in the tank. The zones of high mixing are zones of high oxygenation and the zones of low mixing indicates are zones of low oxygenation. The presence of these zones creates the heterogeneous aerobiosis regime in the cultures inside the tank.

In one example of a feeding system, there are nine production fermentation tanks organized in 3 parallel rows or sets of the 3 bioreactors and there are three hot liquid feed (HLF) lines: one feed line for each set of three bioreactors, such as that shown in FIG. 27. This configuration allows for the feeding of multiple bioreactors simultaneously in parallel. The lines connecting the storage vessels to the valve bank are equipped with a pump or a pressurized line and a flow transmitter to monitor and control the flow rate of concentrated media ingredients in the line. The flow transmitter monitors the feed medium flow rate and controls the pump as required. Accurate monitoring and control of the fluid transfers allows the critical delivery of an accurate volume of each concentrated media ingredient to the cultures growing in the bioreactors. Each of the concentrated media ingredients connects to all three HLF transfer lines feeding the sets of bioreactors through double seat valves. The concentrated carbon and concentrated nitrogen sources are transferred from the trace tanks to the valve bank by headspace pressurization and/or via the pump.

### Example 13: Large-Scale Production and Sparger Testing

A modified growth medium was used to grow Euglena gracilis which was composed of (in g/L dissolved in microfiltered water): 10 g/L glucose; 5 g/L yeast extract; 2 g/L (NH4)2SO4; 1 g/L KH2PO4; 1 g/L MgSO4; 0.1 g/L CaCl2; 5 mL of Trace salts per 100 L of media which included (g/L): 19.6 g/L FeCl3.6H₂O; 3.6 g/L MnCl2.4H₂O; 2.2 g/L ZnSO4.7H₂O; 0.4 g/L CoCl2.6H₂O; 0.3 g/L Na2MoO4.2H₂O; 10 g/L NaEDTA.2H2O, and 40 mL of vitamin cocktail per 100 L of media which included (in g/L): 25 g/L vitamin B1; 0.125 g/L vitamin B12; 0.005 g/L vitamin B6; 0.00025 g/L vitamin B7. The medium pH is adjusted to 3.2 with either hydrochloric acid or with phosphoric acid.

For the cultivation in the 500 L bubble column bioreactors, 100 L of fresh growth medium was inoculated with approximately 18 to 24 L of inoculum cultures. The starting dry cell weight concentration ranged between 2 and 3 g/L. The culture was incubated at 28° C. and the airflow ranged from 0.2 to 1.5 scfm (5.6 liters/min to 42.5 liters/min). In the 20,000 L bubble column bioreactors, 3700 L of fresh media would be inoculated with 200 to 300 L of inoculum cultures transferred from the 500 L bubble column bioreactors for a total starting culture volume of 3900 to 4000 L. The initial dry cell weight concentration ranged between 3 to 7 g/L. The culture was incubated at 28° C. and the airflow ranged from 6 to 50 scfm (170 liters/min to 850 liters/min).

To verify the impact of growing Euglena gracilis cultures with a fine and a coarse sparger, a 10″ 10 μm-grade sparger was fabricated and angled at 45° down toward the tank bottom and installed in a 20,000 L bubble column bioreactor from which the 3 fine spargers were removed. The new tank/sparger configuration mimicked that of the 500 L bioreactors. At the time of testing, the primary task was to achieve higher biomass productivity for commercial and downstream process development purposes. These trials were therefore integrated to the production schedule

Since the gas entry velocity at the sparger was shown to be the main factor for cell death for 519 and NS0 cell line cultures grown in bioreactors, and that the velocity is proportional to the square root of the differential pressure, the latter is therefore an indirect measurement of the gas entry velocity. An aeration test with the coarse and fine spargers was conducted in the 500 L bioreactors. The results in Table 38 revealed that the pressure drop or pressure differential (ΔP) across the fine spargers at various flow rates was almost twice that of the coarse spargers. This suggests that the gas entry velocity through the fine sparger pores is approximately two-fold higher than the velocity through the coarse sparger pores.

Despite the greater surface exchange area (1.35×) of the fine spargers (0.5 um) relative to the coarse spargers, air injection in the fine spargers resulted in double the differential pressures at all flow rates. The higher differential pressure indicates that the gas entry velocity through the fine spargers are greater than that through coarse spargers and may explain low productive growth in bioreactors equipped with the fine spargers.

TABLE 38
Pressure differential across a fine and coarse sparger at various air flowrates.

| | 5-7 μm | 0.5 μm |
|---|---|---|
| The following measurements were taken with a | grade” | grade” |
| reactor volume of 125 L (of 450 L working) | or “coarse” | or “fine” |
| Diameter | 0.75 in | 0.75 in |
| Sintered Metal Length | 3.5 in | 4.75 in |
| Effective Surface Area | 8.25 in² | 11.2 in² |
| ΔP at Minimum Flow (inoculation conditions) | 3.35 psi | 7.8 psi |
| ΔP at 0.5 scfm Air Flow | 4.05 psi | 10.15 psi |
| ΔP at 0.75 scfm Air Flow | 4.45 psi | 10.9 psi |
| ΔP at 1.0 scfm Air Flow | 5.25 psi | 12.4 psi |
| ΔP at 1.5 scfm Air Flow | 6.45 psi | 13.3 psi |

Note:
ΔP is pressure drop or pressure differential across the sparger

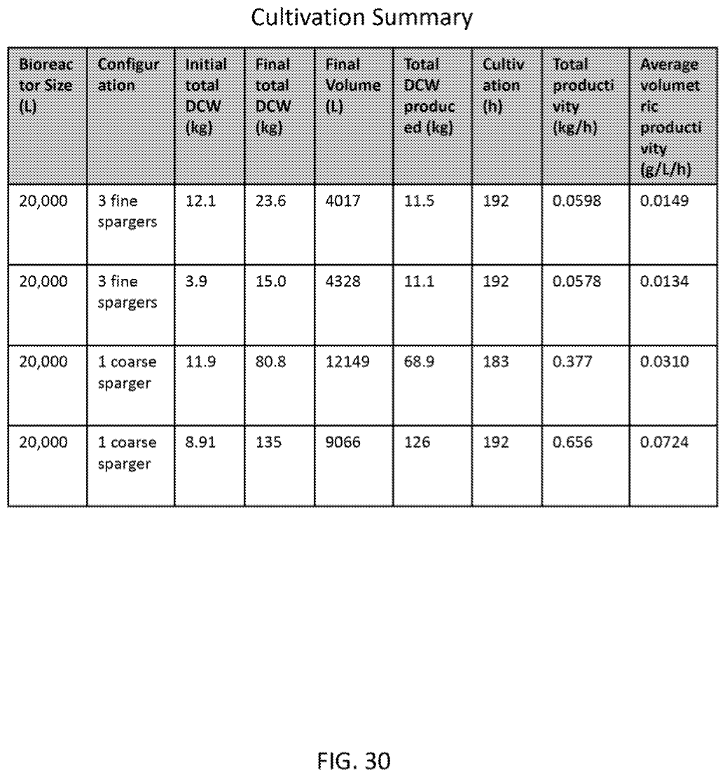

Test cultivations were performed in a 20,000 L bubble column bioreactor in which the coarse sparger was installed and the 3 fine spargers removed. Both cultivations were inoculated with an initial cell concentration of 2.2 g DCW/L and 2.7 g DCW/L respectively (FIG. 30). The total dry cell weight after 192 hrs of cultivation in cultivation reached 135 DCW kg (and 80.8 kg DCW in 183 hours of cultivation respectively following an exponential trend similar to the growth pattern observed in the 500 L bioreactors. In addition, the cell concentration in some runs were 15 g DCW/L and 12.6 g DCW/L. On the other hand, the total dry cell weight of the cultivations in the bioreactors equipped with 3 fine spargers reached a maximum total biomass yield at 23 kg and 14.9 kg respectively after 192 hours of cultivation and the maximum cell concentration reached 5.8 g DCW/L and 3.46 g DCW/L respectively.

Cell growth in bioreactors equipped with 1 single coarse sparger surpassed that in the cultivations with the 3 fine spargers in which all 3 fine spargers were in use after 120 hours of cultivation. This result is three to five times more productive than with the three fine air sparger configurations based on the average volumetric biomass productivity. The average volumetric productivities was in the bioreactors equipped with the fine spargers were 0.0149 and 0.0134 g/L/h respectively compared to 0.0724 g/L/h for cultivation in the bioreactor equipped with 1 coarse sparger. This represents a 5.4-fold increase in the average volumetric productivity. Moreover, the volumetric productivity of cultivation in the 20,000 L bioreactors equipped with the coarse sparger was similar to those in the 500 L bioreactors which indicates a successful fermentation scale-up. FIG. 30 is a table showing an example cultivation result according to this example, showing improved results when using the coarse sparger.

### Example 14: Additional Scale Up

Production Method Overview

Euglena gracilis biomass is to be generated in a large-scale production fermenter by batch cultivation. The overall cultivation procedure includes 2 initial cell expansion steps in 3 L shake flasks, and then in a seed (300 L) fermentor, and in a batch cultivation in a 7000 L fermenter thereafter. Please see Table 39 below for a general description of the cultivation method.

TABLE 39
Cultivation Method Overview

| Cultivation Method | Description |
|---|---|
| Shake flask (3 L) | Use six “Euglena gracilis” agar slants to seed 1 L |
| Operation mode: | of growth medium in a 3-litre non-baffled shake |
| BATCH | flask and incubate for 2 days at 28° C. and 100 |
| Duration: 2 days | rpm (on an orbital shaker) in the dark. |
| Target Density: | |
| 4-7 g DCW/L | |
| Shake flask (3 L) | Use 100 mL inoculum aliquotes from the previous |
| Operation mode: | 1 L shake flask to seed 1 L of growth medium in |
| BATCH | each of ten 3-litre non-baffled shake flasks and |
| Duration: 2 days | incubate for 2 days at 28° C. and 100 rpm (on an |
| Target Desinty: | orbital shaker) in the dark. |
| 4-7 g DCW/L | |
| Seed (300 L) | Start culture at 53% of working volume (200 L) |
| Fermentation | Pulse concentrated media feed (constant rate) |
| Operation mode: | Continuously feed to 90% of max working |
| FED-BATCH | volume and transfer to production fermentor |
| Duration: 5 days | pH = 3.25 and Temperature = 28° C. |
| Target Density: | |
| 13-26 g DCW/L | |
| Production (7000 L) | Start with the target volumn of inoculated broth. |
| Fermentation | Leave culture to grow for 2 to 3 days with NO |
| Operation mode: | feeding |
| BATCH | pH = 3.25 and Temperature = 28° C. |
| Duration: 2-3 days | Harvest |
| Target Cell Density: | |
| 5-10 g DCW/L | |

1. Initial Growth Step—Shake Flask Seed Culture

The shake flask (SF) step comprises 2 growth cycles in 3 L non-baffled shake flasks and requires the use of an orbital shaker. The first SF growth cycle is to be incubated for 48 hours under conditions listed in Example 3, and second SF growth cycle with 10 SFs is to be implemented for 48 hours also similar conditions. A total of twelve (12) 3 L non-baffled SFs with vented lids are required for this step.

2.1 General Procedure

This growth step consists of a fed-batch cultivation to be implemented as per operation parameters listed in Table 40. The feed rate schedule is to be implemented with the Noblegen online feed calculator. The Noblegen feed rate calculator takes values from the sample entries operators enter through a webpage. This is done every 8 hours of a batch and calculates the next appropriate feed rate for the associated vessel. This is based on a mathematical formula that is optimized for the growth of Euglena gracilis determined in house. The values utilized in this calculation are dry cell weight, total volume and the residual glucose. The website then instructs operators what the appropriate feed rate should be for the next feed.

TABLE 40
Fed-batch fermentation Operation Specifications for
Seed Fermenters (300 L)

| Seed process details | Acceptable Range | Target |
|---|---|---|
| Inoculum relative volume | 1-10% | >2% |
| Initial culture volume | 100-110 L | 105 L |
| Final culture volume | 175-185 L | 180 L |
| Feeding trigger | At 24 hours | At 24 hours |
| Feed rate | Use feed | Use feed calculator |
| | calculator | |
| Initial glucose concentration | 13-17 g/L | 15 g/L |
| Initial cell density | >=0.5 g DCW/L | 1.6-3.2 g DCW/L |
| Final cell density | 20-26 g DCW/L | >20 g DCW/L |
| pH Control | 3.0-3.4 | 3.25 |
| Dissolved Oxygen | 0.5 ppm-1 ppm | 0.5 ppm-1 ppm |
| Temperature | 27° C.-29° C. | 28° C. |
| Headspace pressure | 3.2 psi-3.8 psi | 3.5 psi |
| Airflow Rate Range | 0.1-0.4 vvm | 0.1-0.4 vvm |
| Agitation (rpm) | 20-180 | 20-180 |
| | (1 impeller) | |
| Duration of growth cycle | 5-6 days | 5 days (120 h) |
| | (120 h-144 h) | |
| Samples | | 0, 24, 48, 72, |
| | | 96, and 120 h |
| Analytical testing | | Microscopy |
| (per sample) | | Purity Testing |
| | | (Culture streaking on |
| | | Tryptic Soy Agar plates) |
| | | Dry Cell Weight |
| | | (concentration) |

The control of the pH can be accomplished with 1 mol/L (40 g/L) sodium hydroxide. The seed cultivation time may range from 5 to 6 days depending on the initial wet cell weight achieved.

Sample Analytics Reporting

All analytical results from the samples and pictures are to be uploaded onto the database.

2.2 Growth Media

The growth medium formulation is shown in Table 41.

TABLE 41
(Starting) Growth medium formulation for Seed Cultivation

| Growth Media | Formulation |
|---|---|
| Starting Media | 16.5 g/L dextrose monohydrate |
| Formulation (105 L) | 5 g/L Yeast Extract |
| | 2 g/L Ammonium sulfate |
| | 1 g/L Monopotassium phosphate |
| | 1 g/L Magnesium sulfate heptahydrate |
| | 0.1 g/L Calcium sulfate anhydrous |
| | 1 g/L Vegetable oil |
| | Trace Metals solution (0.05 mL/L culture) |
| | Vitamin cocktail (0.4 mL/L culture) |
| | 85% Phosphoric acid to adjust the pH |
| | to 3.25 (1.5 mL/L) |

The concentrated feed medium formulation to be used for the intermediate fermentation is described in Table 42 below.

TABLE 42
Concentrated feed medium formulation for seed (300 L)
fermentation (only)

| Growth Media | Formulation |
|---|---|
| Concentrated Media | 111.5 g/L dextrose monohydrate |
| Formulation (75 L) | 35 g/L Yeast Extract |
| | 14 g/L Ammonium sulfate |
| | 7 g/L Monopotassium phosphate |
| | 7 g/L Magnesium sulfate heptahydrate |
| | 0.7 g/L Calcium sulfate anhydrous |
| | Trace Metals solution (0.35 mL/L feed) |
| | Vitamin cocktail (2.8 mL/L feed) |

3. Final Growth Step—Batch Fermentation

3.1 Fermentation Process Specifications

The large scale batch production of Euglena gracilis is to be implemented to achieve the required cell density as per the operation parameters in Table 43. The duration of this growth cycle is 2 to 3 days. No pH control is required for this step.

TABLE 43
Fermentation Operation Specifications for batch
cultivation (Production Fermentor)

| Production | | |
|---|---|---|
| process details | Acceptable Range | Target |
| Initital culture volume | 70% to 80% max. | 75% to 80% of max |
| | Working volume | working volume |
| Final culture volume | 70% to 80% max. | 75% to 80% of max |
| | Working volume | working volume |
| Feeding trigger | NO FEED | NO FEED |
| Initial glucose | 13-17 g/L | 15 g/L |
| concentration | | |
| Initial cell density | >0.64 g DCW/L | 1.6-3.2 g DCW/L |
| Final cell density | 5-10 g DCW/L | 7 g DCW/L |
| Initial pH (no control) | 3.0-3.4 | 3.25 |
| Dissolved Oxygen | 0.5 ppm-0.8 ppm | 0.5 ppm-0.8 ppm |
| | (5%-8%) | (5%-8%) |
| Temperature | 26° C.-30° C. | 28° C. |
| Headspace pressure | 3.2 psi-3.8 psi | 3.5 psi |
| Airflow Rate Range | 0.1-0.4 vvm | 0.1-0.4 vvm |
| Agitation (stirring) rate | 20-180 | 20-180 |
| | (1 impeller) | |
| Incubation duration | 48 h to 72 hours | 48 hours |
| Samples | | 0 h, 12 h, 24, 36 h, |
| | | and 48 h (60 h and |
| | | 72 h if necessary), |
| | | every 6 hours if needed |
| Analytical testing | | For all samples |
| (per sample) | | Microscopy Inspection |
| | | Purity Testing |
| | | (Culture streaking on |
| | | Tryptic Soy Agar plates) |
| | | Dry Cell Weight |
| | | (concentration) |
| | | Glucose quantification |

Sample Analytics Reporting

All analytical results from the samples and pictures are to be uploaded onto the database for analysis.

3.2 General Procedure

3.2.1 Growth Medium Formulation

The growth medium to be used for the production of Euglena biomass is shown in Table 44. Please note that the formulation of the starting medium in this step contains 3 g/L of yeast extract and 1.2 g/L of ammonium sulfate (instead of 5 g/L yeast extract and 2 g/l ammonium sulfate as in the previous growth steps). This allows the optionality to further increase protein or paramylon yield by increasing or decreasing the nitrogen sources during the feed, if needed.

TABLE 44
(Starting) Growth medium formulation for the Large-Scale batch cultivation

| Growth Media | Formulation (1X broth) |
|---|---|
| Starting Media | 16.5 g/L dextrose monohydrate |
| Formulation | 3 g/L Yeast Extract |
| | 1.2 g/L Ammonium sulfate |
| | 1 g/L Monopotassium phosphate |
| | 1 g/L Magnesium sulfate heptahydrate |
| | 0.1 g/L Calcium sulfate anhydrous |
| | 1 g/L Vegetable oil |
| | Trace Metals solution (0.05 mL/L culture) |
| | Vitamin cocktail (0.4 mL/L culture) |
| | 85% Phosphoric acid to adjust the pH to 3.25 |
| | (1.5 mL/L) |

The above growth medium has been formulated to meet the target product specifications shown in Table 44.

3.2.3 Inoculation Guidelines

The inoculum culture is to be transferred from the seed fermenter to the production fermenter. The seed culture should be well mixed during its transfer from the seed fermenter to the production fermenter to avoid excessive cell settling and uneven cell flow. The broth receiving the inoculum should be pre-warmed to the specified temperature and fully saturated with dissolved oxygen. The volume of the inoculum should be 5% to 10% by volume.

3.2.4 Sampling and Analytical Testing

3.2.4.1 Frequency and Required Sample

Sampling of the culture is to be performed every 6-12 hours (at a minimum), for example during culturing at 0, 6, 12, 18, 24, 30, 36, 42, and 48 hours with 2×50 mL samples at each time point. As well, at the end of the batch i.e. 48 hours, 2×2 L sample is taken. All analytical results from each samples and pictures are to be uploaded onto the database. The two 50 mL samples are to be collected at each time point: one sample should be processed for immediate testing and the second (duplicate) sample should be frozen immediately to be sent back for external analysis. Samples are to be tested for purity, cell density via cell dry determination, and for fermentation metabolites tracking. Metabolites to be analyzed include, but are not limited to: Glucose, potassium, calcium, sulfate, phosphate, succinate, lactate. Glucose is always measured.

3.2.5 Harvest Guidelines

3.2.5.1 Process Description

Following the completion of fermentation, the broth is chilled to 15° C. using chilled water circulation through the fermenter jacket. If the broth is required to sit for >12 hours prior to initiation to downstream processing, then the broth should be batch pasteurized to inactivate the cells. This may be achieved in the fermenter using direct steam injection (final temperature 60° C., 45 psi g steam, 60 min holding time). The heated broth is then chilled to 15° C. using chilled water circulation through the fermenter jacket. Both processes should provide adequate agitation during heat transfer operations. Ideally, the fermentation and harvest should be planned such that batch pasteurization is not necessary.

Chilled broth is transferred to a chilled (15° C.) drop tank, which is subsequently transferred as a batch to the centrifuge feed tank. The broth is then diluted (inline) during centrifuge feeding with municipal water to a final cell density of 10 g-wet/L (roughly 0.32% dry solids). Concentrate collected from the nozzles is sent back to the centrifuge feed tank, forming a recirculation loop until a target concentrate solids of 5% is achieved in the nozzle stream. 5% solids has been preliminarily selected to provide enough material for pasteurization, as well as limiting concentration build-up within the centrifuge until nozzle performance has been validated. Supernatant and bowl discharges are discharged to the drain.

Concentrated sludge is then forwarded to pasteurization (85° C., 15 sec. hold time). All material is forwarded to the pasteurizer waste tank, with final product samples (see schedule in Section 3.2.5.2) collected from the pasteurizer discharge sample port. Collected final product can be sent for drying i.e. Spray drying, drum drying or other acceptable means of drying.

3.2.5.2 Sampling Requirements

Various samples are required during the downstream process to (1) confirm product and process quality in real time and (2) provide samples for analytics for in-house final product quality testing. Real time sampling required during operation includes:
Moisture analysis (infrared balance or similar)
Microscopy analysis (20-40× magnification)

Additional samples are required to be taken at defined process points and immediately frozen for shipping following trial completion. A preliminary sampling matrix includes:
2×20 L bucket; nozzle centrifuge sludge, pre-pasteurization (frozen, shipped, and then dried at location)
2×20 L bucket; nozzle centrifuge sludge, post-pasteurization (frozen, shipped, and then dried at location)
4×50 mL Falcon tubes; nozzle centrifuge sludge, pre-pasteurization (frozen and shipped to location)
4×50 mL Falcon tubes; nozzle centrifuge sludge, post-pasteurization (frozen and shipped to the location)
4×50 mL Falcon tubes; nozzle centrifuge supernatant (frozen and shipped to the location)
4×50 mL Falcon tubes; final fermentation broth (frozen and shipped to the location)

Results and Discussion:

2 Step Shake Flask Results:

In the first step, after 48 hours at 28 C, the DCW was 5.15 g/L and it had used approximately 7.27 g or 44% of the glucose. The second step had an increased glucose consumption of 8.94 g/L glucose or 54.2%. After the second step, the flasks were pooled and used to inoculate the seed fermentor which had a total volume of 10 L and a DCW of 18.24 g/L. The final glucose level also dropped to 2.89 g/L, or a 82.5% glucose consumption.

300 L Tank Seed Fermentation:

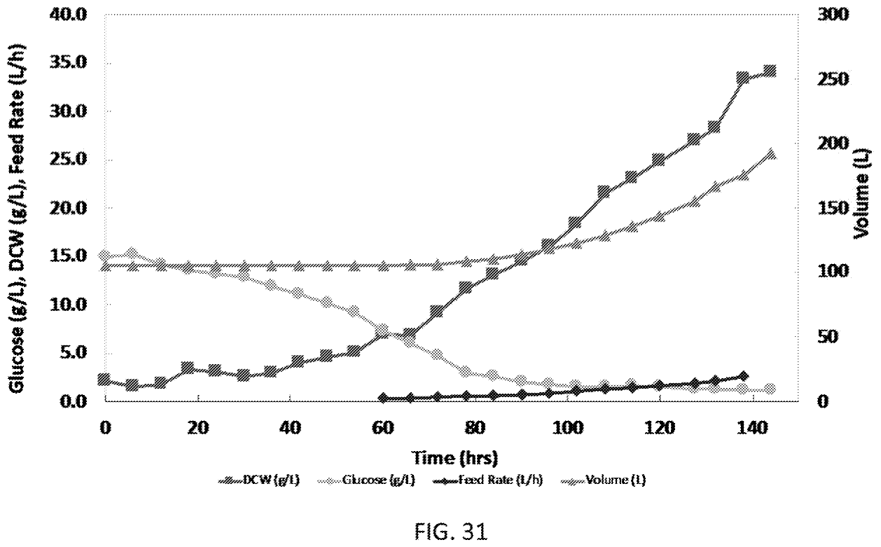

Fermentation in the 300 L tank was cultured at 28 C, pH 3.25, 15% dissolved oxygen (DO, ppm), with an initial glucose level of 15.2 g/L and an initial airflow rate of 10.5 (slpm). Stir rate was between 60-120 rpm. Summary of the 5 day fermentation metrics can be found in Table 45 and FIGS. 31-33. In Table 45 below, the fermentation metrics of the run are displayed. Productivity was calculated on the batch phase, which was the first 60 hours of the run, fed batch phase which was the remainder of the run and overall productivity which is based on the change in DW (g) over the final volume (L) divided by the change in time (h). Yields based on glucose, RM (Raw Materials) and oxygen were in the range of historical data.

TABLE 45
Fermentation metrics run in a 300 L tank. Metrics measured included:
time, final DCW, final volume, total DCW generated, glucose consumed,
oxygen consumed, total RM fed, yields, and productivity.

| 300 L (192 L) Fermentation Metrics | Values |
|---|---|
| Duration of cultivation step = | 144 h |
| Initial DCW = | 2.2 g/L |
| Final DCW = | 34.0 g/L |
| Initial Volume = | 105 L |
| Final Volume = | 192 L |
| Total DCW generated = | 6389 g |
| Total glucose consumed = | 10571 g |
| Total oxygen consumed = | 3242 g |
| Total RM fed = | 14795 g |
| Yield (biomass/glucose) = | 0.604 |
| Yield (biomass/RMs) = | 0.369 |
| Yield (biomass/O2) = | 1.95 |
| Average Batch Phase Productivity (DCW) = | 0.136 g/L/h |
| Avg. Fed-Batch Phase Productivity (DCW) = | 0.493 g/L/h |
| Overall productivity (DCW) = | 0.240 g/L/h |

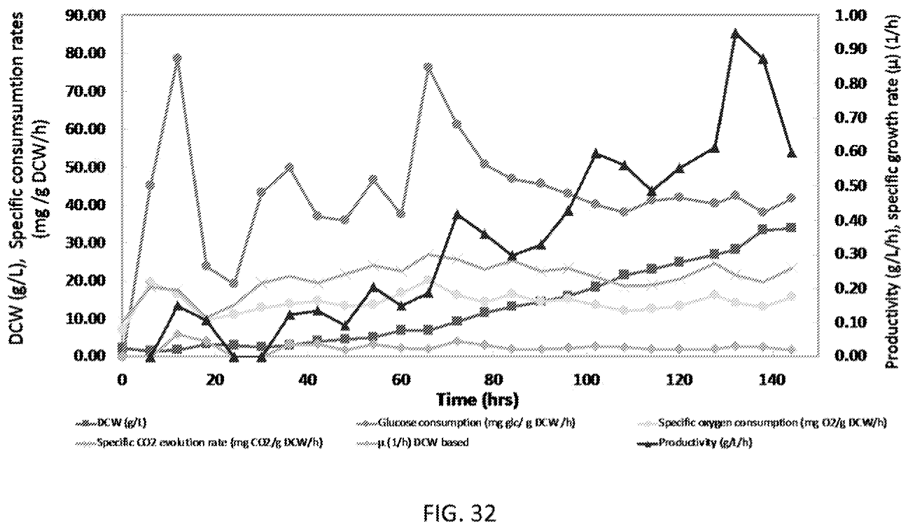

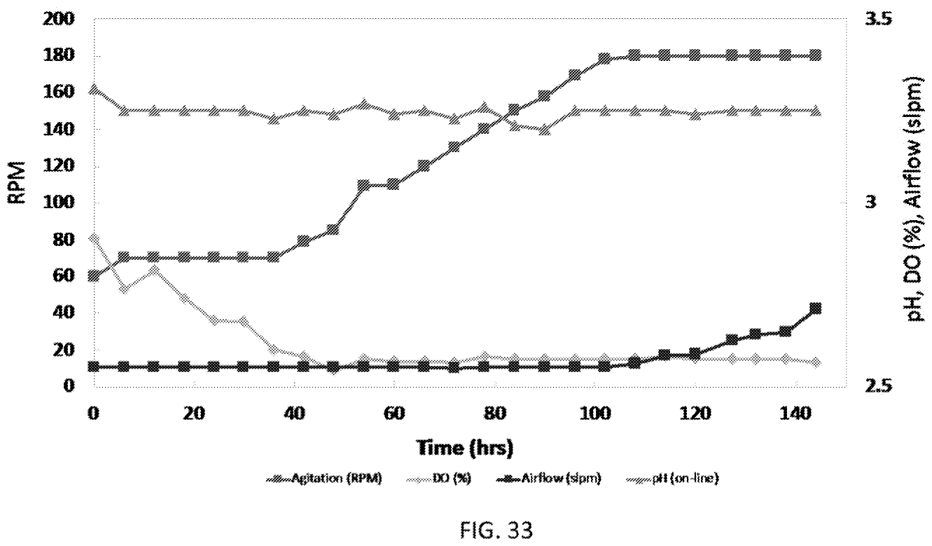

From FIG. 31, the specific growth rate and specific glucose consumption was steady during the fed batch (i.e. after 72 hour mark). Glucose was maintained between 1.2-3 g/L and that the respiration quotient (RQ, produce mol CO₂/consumed mol O₂) was fairly stable (FIG. 32). From FIG. 32, the trend of volumetric productivity is shown to increase with time and is proportional to the total biomass i.e. there was peak productiveness 128 and 144 hours at a peak average of 0.757 g DCW/L/hr. FIG. 33 shows that as the DO % decreased, the agitation increased till a maximum of 180 RPM. Airflow was fairly constant till the end with a slight increase after 100 hours. pH remained fairly constant over the course of the fermentation run.

7000 L Tank Fermentation:

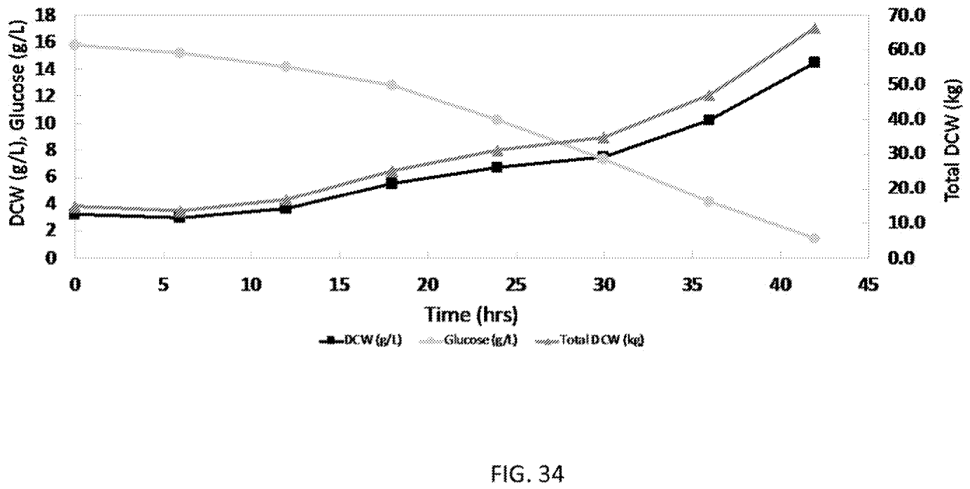

In this run, there was only a short lag time as growth began almost immediately, as seen in FIG. 34. The fermentation metrics of the run in the 7000 L tank is observed in Table 46. The initial pH was set to 3.25 with a DO % of 5-8%, a temperature of 28 C, a starting airflow rate of 1500 slpm, and the initial glucose level was 15 g/L. Yields were slightly higher than historic data but still in comparable levels.

TABLE 46
Fermentation metrics run in a 7000 L tank. Metrics measured
included: time, final DCW, final volume, total DCW generated,
glucose consumed, oxygen consumed, total RM fed, and productivity

| 7000 L (4630 L) Fermentation Metrics | |
|---|---|
| Duration of cultivation step = | 42 h |
| Initial DCW = | 3.2 g/L |
| Final DCW = | 14.5 g/L |
| Initial Volume = | 4633 L |
| Final Volume = | 4585 L |
| Total DCW generated (g) = | 51580 g |
| Total glucose consumed (g) = | 63940 g |
| Total oxygen consumed = | 22990 g |
| Total RM fed (g) = | 99189 g |
| Yield (biomass/glucose) = | 0.807 |
| Yield (biomass/oxygen) = | 2.24 |
| Yield (biomass/RMs) = | 0.520 |
| Average Batch Phase Productivity = | 0.320 g/L/h |
| Overall productivity = | 0.312 g/L/h |

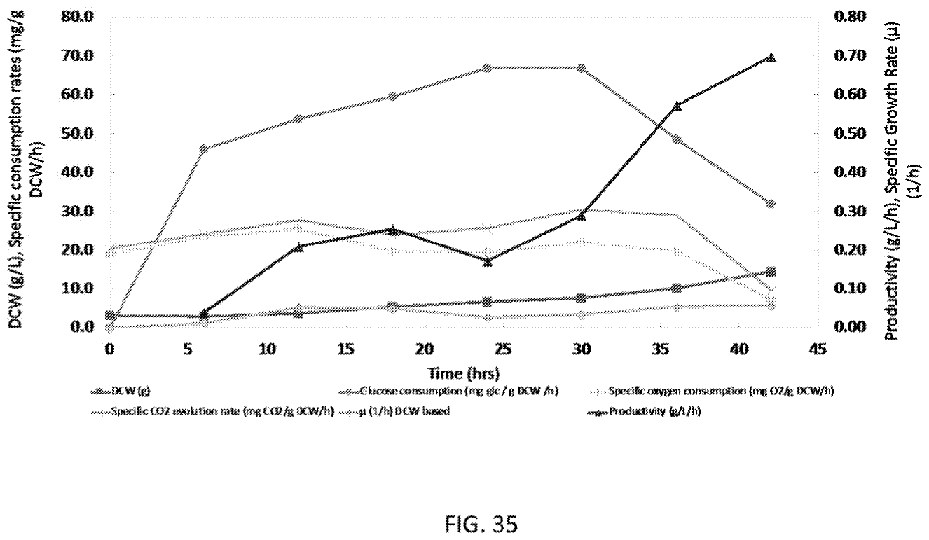

Peak volumetric productivity for this run increased with time and was proportionate to the total biomass, with peak productivities between 30-42 hours (peak average of 0.521 g DCW/L/h (FIG. 35). The overall productivity was 0.312 g/L/h, which was a higher average then in the 300 L run. The RQ was also fairly stable during this run.

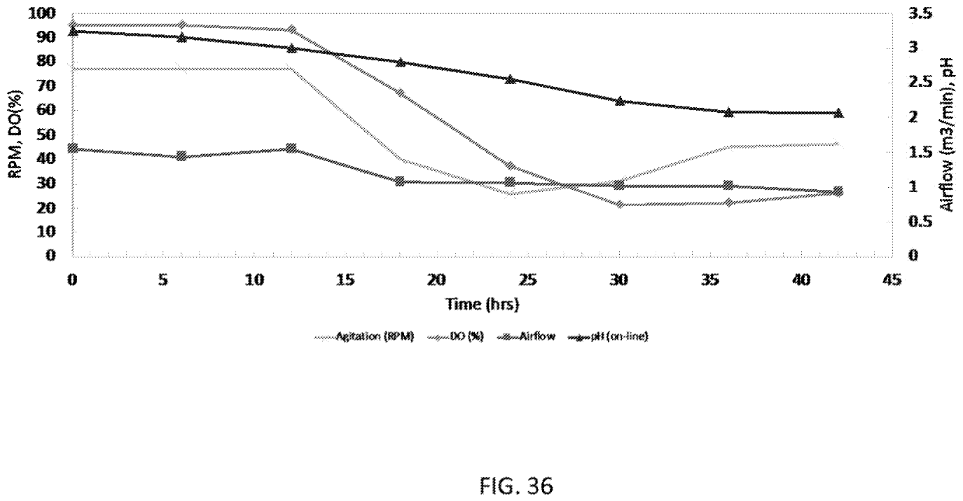

In this fermentation run, agitation was adjusted to be between 20 RPM and 90 RPM as seen in FIG. 36. Airflow was fairly consistent, whereas there was a steady drop over time for the pH and for the DO. Future runs would aim to keep the DO at a higher, consistent level to better oxygen availability to the cells.

Comparison of growth metrics between 300 L and 7000 L scale runs:

Table 47 summaries the specific glucose consumption, specific oxygen consumption, specific CO₂ evolution rate and RQ between the two scale sized runs. This data is used to help predict production yields in the future. The 7000 L had a higher consumption and evolution rate, which was as expected due to the higher biomass generation.

TABLE 47
Consumption summary data 300 and 7000 L fermentation runs.

| | Specific glucose | Specific oxygen | Specific CO₂ | |
|---|---|---|---|---|
| | consumption | consumption | evolution rate | |
| Fermentation | (mg glc/g | (mg O₂/g | mg Co₂/g | |
| Step | DCW/h) | DCW/h) | DCW/h) | RQ |
| 300 L | 44.3 ± 13.1 | 14.6 ± 2.4 | 20.8 ± 3.8 | 1.04 ± 0.15 |
| 7000 L | 53.4 ± 12.5 | 19.5 ± 5.5 | 23.9 ± 2.7 | 0.90 ± 0.12 |

In Table 48, the oxygen uptake and carbon dioxide evolution rates are reported. In general, the oxygen uptake rate helps show the oxygen transfer in the bioreactor systems which can be a metric used to assess the fermentation run feasibility. In general, a lower oxygen uptake number is seen as positive as then there is not a worry about oxygen limitation during cultivation. Carbon dioxide is also useful as an evolution rate, however if the level is too low or too high, it could suggest that cells are not growing optimally if too low, and if too high, it could suggest an abnormal run.

TABLE 48
Comparison between 300 and 7000 L tanks highlighting the
minimum O₂ uptake rate, maximum O₂ uptake rate, median O₂
uptake rate (mmol/L/h), minimum CO₂ evolution rate,
Maximum CO₂ evolution rate and Median CO₂
evolution rate (mmol/L/h).

| | Minimum | Maximum | Median O₂ |
|---|---|---|---|
| Fermentation | O₂ Uptake Rate | O₂ Uptake Rate | Uptake Rate |
| Step | (mmol/L/h) | (mmol/L/h) | (mmol/L/h) |
| 300 L | 0.463 | 16.6 | 4.62 |
| 7000 L | 1.90 | 6.31 | 3.34 |
| | Minimum | Maximum | Median CO₂ |
| Fermentation | CO₂ evolution | CO₂ evolution | evolution Rate |
| Step | Rate (mmol/L/h) | Rate (mmol/L/h) | (mmol/L/h) |
| 300 L | 0.450 | 18.0 | 5.32 |
| 7000 L | 1.49 | 6.78 | 3.09 |

Conclusions:

This example highlights the use of the process at another facility, and with the use of mechanical agitations. It was successfully scaled up from slant cultures to 7,000 L tank run. There was higher volumetric productivity at the end of cultivation when there were higher cell densities. The specific glucose and O₂ consumption was fairly consistent throughout the cultivations, as well as the specific CO₂ generation. Growth profiles were also similar to historical data.

To test a mock of a larger scale fermentor transfer, the inoculum was transferred from the 7000 L fermentor to a 128,000 L scale fermentor. Visual observations showed healthy cells after being pressurized and transferred to a centrifuge, with little cell lysis. Harvesting was tested with a nozzle type disc stack centrifuge with a bowl speed of 4400 rpm, back pressure of 65 psig, discharger interval of 60 mins, feed rate of 420-640 L/min, feed temp of 9-11° C. with 10, 1.2 mm nozzles, and 5 blanks. And online water wash was added in a 3:1 ratio. Collected harvest shows that there was lysis of the cells that led most likely to a rise in pH of the culture from 2.06 to 5.57, and the solids concentration doubled. Pasteurization was also tested through a continuous HTST pasteurizer with a flow rate of 68 L/min, hold temperature of 85° C. for 2 minutes and a cooling temperature of 10° C. There were no issues such as plugging or cooking observed during operations.

The disclosures of each and every patent, patent application, publication, and accession number cited herein are hereby incorporated herein by reference in their entirety.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

1.-57. (canceled)

58. A method of heterotrophically culturing a microorganism comprising:
culturing the microorganism in a culture media containing one or more carbon source, one or more nitrogen source, one or more sugar, one or more alcohol, one or more oil, and one or more salt;
maintaining a pH of between about 2.0 to about 4.0;
maintaining a temperature of about 20° C. to about 30° C.; and
maintaining an environment with substantially no light;
wherein the culturing occurs within a tank configured to receive the culture media, an air supply system configured to introduce a gas into the tank, an ability to mix the culture media and microorganisms within the tank, wherein the air supply system includes a lower pressure supply device and a higher pressure supply device.

59.-68. (canceled)

69. The method of claim 58, wherein the gas is selected from the group consisting of compressed air, oxygen, nitrogen, helium, and combinations thereof.

70. The method of claim 58, wherein the lower pressure supply device is a sparging stone having a pore size of less than 30 microns,
wherein the higher pressure supply device includes at least one spray nozzle configured to direct a stream of gas into the tank and configured to pivot to change the direction of the stream of gas,
wherein the stream of gas is supplied at a rate of about 0.1 L/minute, and
wherein the higher pressure supply device and the lower pressure supply device are independently electronically controllable.

71. (canceled)

72. (canceled)

73. The method of claim 58, wherein the air supply system is configured to simultaneously create a plurality of zones within the tank, and wherein the plurality of zones include at least one aerobic zone and at least one anaerobic zone.

74. (canceled)

75. The method of claim 58, wherein the tank has at least a 3:1 height-to-diameter size ratio.

76. The method of claim 58, wherein the tank has a capacity of about 10 liters to about 1,000,000 liters.

77. (canceled)

78. The method of claim 58, wherein the microorganism is selected from the group consisting of Euglena gracilis, Euglena sanguinea, Euglena deses, Euglena mutabilis, Euglena acus, Euglena viridis, Euglena anabaena, Euglena geniculata, Euglena oxyuris, Euglena proxima, Euglena tripteris, Euglena chlamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabrica, Euglena granulata, Euglena obtusa, Euglena limnophila, Euglena hemichromata, Euglena variabilis, Euglena caudata, Euglena minima, Euglena communis, Euglena magnifica, Euglena terricola, Euglena velata, Euglena repulsans, Euglena clavata, Euglena lata, Euglena tuberculata, Euglena cantabrica, Euglena acusformis, Euglena ostendensis, Chlorella autotrophica, Chlorella colonials, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, Chlorella vulgaris, Schizochytrium aggregatum, Schizochytrium limacinum, Schizochytrium minutum, and combinations thereof.

79. The method of claim 58, wherein the tank has a monitoring system that measures a parameter selected from the group consisting of pH, dissolved oxygen, cell density, lumen level, glucose level, temperature, culture volume in the bioreactor, nitrogen levels (e.g. ammonium, glutamate), media composition, residual molecular oxygen in bioreactor exhaust gas, carbon dioxide levels in bioreactor exhaust gas, and combinations thereof.

80. The method of claim 58, wherein the lower pressure supply device comprises a plurality of spargers including at least one sparger having a first pore size and at least one sparger having a second pore size,
wherein the second pore size is larger than the first pore size, and
wherein the first pore size is approximately 5-10 microns and the second pore size is approximately 20-70 microns.

81. (canceled)

82. The method of claim 80, wherein the plurality of spargers are positioned in layers that extend in different directions within the tank.

83. The method of claim 82, wherein the layers form a grid near the bottom of the tank.

84. The method of claim 58, wherein the microorganism has a maximum growth rate _{(μmax)} of 0.001-0.1 h⁻¹.

85. The method of claim 58, wherein the culture media turns over up to 300 times in 75 days of said culturing.

86.-89. (canceled)

90. The method of claim 58, wherein the culture media maintains a conversion efficiency of 15% to about 75%.

91. The method of claim 58, wherein the culture medium of cultured microorganism has a specific glucose consumption rate of about 30-75 mg/glc/gDCW/h.

92. The method of claim 58, wherein the culture medium has a dissolved oxygen (DO) value of about 15 to about 100%.

93. The method of claim 58, wherein the culture medium of cultured microorganism has an oxygen uptake rate of about 0.1-40 mmol/L/h.

94. The method of claim 58, wherein the culture medium of cultured microorganism has a specific oxygen consumption rate of about 10-30 mg O₂/g DCW/h.

95. The method of claim 58, wherein the culture medium of cultured microorganism has a specific CO₂ evolution rate of about 10-40 mg CO₂/gDCW/h.

96. The method of claim 58, wherein the culture medium of cultured microorganism has a CO2 evolution rate of about 0.1-40 mmol/L/h.

97. The method of claim 58, wherein the culturing occurs in three cultivation stages: a first stage cultivation, a second stage cultivation, and a third stage cultivation.

98. The method of claim 97, wherein the first stage of cultivation has a productivity of about 0.1 gDCW/L/h to about 0.3 gDCW/L/h.

99. The method of claim 97, wherein the second stage of cultivation has a productivity of about 0.5 gDCW/L/h to about 0.8 gDCW/L/h.

100. The method of claim 97, wherein the third stage of cultivation has a productivity of about 0.4 gDCW/L/h to about 3.0 gDCW/L/h.
